SEQUENCE LISTING (63 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDRH3
  SITE 1: note = MISC_FEATURE - Xaa denotes Phe, Met, Gln, Leu or Val
  SITE 6: note = MISC_FEATURE - Xaa denotes Ser or Ala
  SITE 7: note = MISC_FEATURE - Xaa denotes Phe, Leu, Ala or Met
  SITE 9: note = MISC_FEATURE - Xaa denotes Tyr, Gln, Lys, Arg, Trp, His, Ala, Thr, Ser, Asn orGly
XPNWGXXDX

SEQ ID NO: 4 - protein, 9 aa, synthetic construct
  REGION 1..9: note = CDR H3 SEQUENCE
FPNWGALDQ

SEQ ID NO: 5 - protein, 9 aa, synthetic construct
  REGION 1..9: note = CDR H3 SEQUENCE
VPNMGSLDT

SEQ ID NO: 6 - protein, 9 aa, synthetic construct
  REGION 1..9: note = CDR H3 SEQUENCE
FPNWGSMDA

SEQ ID NO: 7 - protein, 9 aa, synthetic construct
  REGION 1..9: note = CDR H3 SEQUENCE
FPNWGSLDH

SEQ ID NO: 8 - protein, 9 aa, synthetic construct
  REGION 1..9: note = CDR H3 SEQUENCE
MPNWGSFDY

SEQ ID NO: 9 - protein, 9 aa, synthetic construct
  REGION 1..9: note = CDR H3 SEQUENCE
MPNWGSFDT

SEQ ID NO: 10 - protein, 9 aa, synthetic construct
  REGION 1..9: note = CDR H3 SEQUENCE
MPNWGSLDH

SEQ ID NO: 11 - protein, 9 aa, synthetic construct
  REGION 1..9: note = CDR H3 SEQUENCE
MPNWGSFDS

SEQ ID NO: 12 - protein, 9 aa, synthetic construct
  REGION 1..9: note = CDR H3 SEQUENCE
MPNWGSLDT

SEQ ID NO: 13 - protein, 9 aa, synthetic construct
  REGION 1..9: note = CDR H3 SEQUENCE
MPNWGSLDA

SEQ ID NO: 14 - protein, 9 aa, synthetic construct
  REGION 1..9: note = CDR H3 SEQUENCE
MPNWGSLDN

SEQ ID NO: 15 - protein, 9 aa, synthetic construct
  REGION 1..9: note = CDR H3 SEQUENCE
MPNWGALDS

SEQ ID NO: 16 - protein, 9 aa, synthetic construct
  REGION 1..9: note = CDR H3 SEQUENCE
MPNWGSLDN

SEQ ID NO: 17 - protein, 9 aa, synthetic construct
  REGION 1..9: note = CDR H3 SEQUENCE
MPNWGSLDY

SEQ ID NO: 18 - protein, 9 aa, synthetic construct
  REGION 1..9: note = CDR H3 SEQUENCE
MPNWGSFDH

SEQ ID NO: 19 - protein, 9 aa, synthetic construct
  REGION 1..9: note = CDR H3 SEQUENCE
MPNWGSLDS

SEQ ID NO: 20 - protein, 9 aa, synthetic construct
  REGION 1..9: note = CDR H3 SEQUENCE
MPNWGSLDG

SEQ ID NO: 21 - protein, 9 aa, synthetic construct
  REGION 1..9: note = CDR H3 SEQUENCE
VPNWGSLDG

SEQ ID NO: 22 - protein, 28 aa, synthetic construct
  REGION 1..28: note = CDR H3 SEQUENCE
CARFPNWGSLDHWGQGTLVTVSSASIKG

SEQ ID NO: 23 - protein, 28 aa, synthetic construct
  REGION 1..28: note = CDR H3 SEQUENCE
CARMPNWGSLDHWGQGTLVTVSSASTKG

SEQ ID NO: 24 - protein, 28 aa, synthetic construct
  REGION 1..28: note = CDR H3 SEQUENCE
CARMPNWGSFDYWGQGTLVTVSSASIKG

SEQ ID NO: 25 - protein, 12 aa, synthetic construct
  REGION 1..12: note = CDR H3 SEQUENCE
VRMPNWGSLDHW

SEQ ID NO: 26 - protein, 27 aa, synthetic construct
  REGION 1..27: note = CDR H3 SEQUENCE
VRMPNWGSLDHWGQGTLVTVSSADIKG

SEQ ID NO: 27 - protein, 27 aa, synthetic construct
  REGION 1..27: note = CDR H3 SEQUENCE
ARMPNWGSLDHWGQGTLVTVSSASIKG

SEQ ID NO: 28 - protein, 25 aa, synthetic construct
  REGION 1..25: note = CDR H3 SEQUENCE
FPNWGSFDYWGQGTLVTVSSASIKG

SEQ ID NO: 29 - protein, 9 aa, synthetic construct
  REGION 1..9: note = CDR H3 SEQUENCE
VPNWGSLDA

SEQ ID NO: 30 - protein, 9 aa, synthetic construct
  REGION 1..9: note = CDR H3 SEQUENCE
FPNWGSFDY

SEQ ID NO: 31 - protein, 12 aa, synthetic construct
  REGION 1..12: note = CDRL1
RASQSISSSYLA

SEQ ID NO: 32 - protein, 7 aa, synthetic construct
  REGION 1..7: note = CDRL2
GASSRAT

SEQ ID NO: 33 - skipped

SEQ ID NO: 34 - protein, 5 aa, synthetic construct
  REGION 1..5: note = CDRL3 SEQUENCE
QRYAT

SEQ ID NO: 35 - protein, 5 aa, synthetic construct
  REGION 1..5: note = CDRL3 SEQUENCE
QRYST

SEQ ID NO: 36 - protein, 5 aa, synthetic construct
  REGION 1..5: note = CDRL3 SEQUENCE
QMYST

SEQ ID NO: 37 - protein, 5 aa, synthetic construct
  REGION 1..5: note = CDRL3 SEQUENCE
QQVGT

SEQ ID NO: 38 - protein, 5 aa, synthetic construct
  REGION 1..5: note = CDRL3 SEQUENCE
QQVST

SEQ ID NO: 39 - protein, 5 aa, synthetic construct
  REGION 1..5: note = CDRL3 SEQUENCE
QQYST

SEQ ID NO: 40 - protein, 5 aa, synthetic construct
  REGION 1..5: note = CDRL3 SEQUENCE
QSYST

SEQ ID NO: 41 - protein, 5 aa, synthetic construct
  REGION 1..5: note = CDRL3 SEQUENCE
QQYAT

SEQ ID NO: 42 - protein, 5 aa, synthetic construct
  REGION 1..5: note = CDRL3 SEQUENCE
QQYSS

SEQ ID NO: 43 - protein, 5 aa, synthetic construct
  REGION 1..5: note = CDRL3 SEQUENCE
QTYST

SEQ ID NO: 44 - protein, 5 aa, synthetic construct
  REGION 1..5: note = CDRL3 SEQUENCE
QQYGS

SEQ ID NO: 45 - protein, 5 aa, synthetic construct
  REGION 1..5: note = CDRL3 SEQUENCE
QQYAS

SEQ ID NO: 46 - protein, 5 aa, synthetic construct
  REGION 1..5: note = CDRL3 SEQUENCE
QQYEA

SEQ ID NO: 47 - protein, 5 aa, synthetic construct
  REGION 1..5: note = CDRL3 SEQUENCE
QQYET

SEQ ID NO: 48 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 61 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0232585 A1
(43) Pub. Date: Aug. 13, 2026

(54) HIGH CONCENTRATION ANTI-IL13R ANTIBODY FORMULATION

(71) Applicant: Aslan Pharmaceuticals Pte Ltd, Singapore (SG)

(72) Inventors: Alan LUK, San Mateo, CA (US); Danny CHOU, Lomita, CA (US)

(21) Appl. No.: 19/106,557

(22) PCT Filed: Aug. 28, 2023
(86) PCT No.: PCT/SG2023/050586

§ 371 Date: Feb. 25, 2025

Related U.S. Application Data
(60) Provisional application No. 63/373,566, filed on Aug. 26, 2022.

(30) Foreign Application Priority Data
Dec. 15, 2022 (SG) 10202260455R
Apr. 25, 2023 (SG) 10202301153T

(51) Int. Cl. A61K 9/08; A61K 47/18; A61K 47/22; A61K 47/26; C07K 16/28

(52) U.S. Cl. CPC A61K 9/08; A61K 47/183; A61K 47/22; A61K 47/26; C07K 16/2866; C07K 2317/24; C07K 2317/565; C07K 2317/92; C07K 2317/94

(57) ABSTRACT

A stable high concentration formulation of an anti-IL 13R antibody or antigen binding fragment thereof. Also provided is the use of this formulation for treatment, for example for treating inflammation or an autoimmune disease, such as atopic dermatitis.

The present disclosure relates to a high concentration formulation of an anti-IL13R antibody or antigen binding fragment thereof, a method of treatment using the formulation in particular a condition disclosed herein, and a process of making the formulation.

### BACKGROUND

IL-13 has been associated with various conditions, including but not limited to various respiratory and allergy-mediated disorders, fibrosis, scleroderma, inflammatory bowel disease and certain cancers; see, e.g., Wynn, T. A., 2003 Annu. Rev. Immunol. 21:425-456; Terabe et al, 2000 Nat. Immunol. 1 (6): 515-520; Fuss et al, 2004 J. Clin. Invest. 113 (10): 1490-1497; Simms et al, 2002 Curr. Opin. Rheumatol. 14 (6):717-722; and Hasegawa et al, 1997 J. Rheumatol. 24 (2): 328-332. Thus, IL-13 is an attractive target for the treatment of such diseases.

One possible way to inhibit the activity of IL-13 would be to interfere with the binding of IL-13 to its receptor IL-13R, for example by using an antibody specific to IL-13R, such as an antibody specific to IL-13RαI. An effective antibody antagonist to IL-13RαI may also interfere with the binding of IL-13 and prevent heterodimerization of IL-4Rα and IL-13RαI. Such an antibody could inhibit signaling of both IL-13 and IL-4 through the type II receptor while sparing IL-4 signalling through the type I receptor. Signalling through the type I receptor is essential in the induction phase of the immune response during which Th2 cells differentiate. T cells do not express IL-13RαI so the type II receptor plays no role in Th2 differentiation. Hence, an IL-13RαI antibody should not affect the overall Th1/Th2 balance. Signalling through the type II IL-4/IL-13 receptor is critical during the effector-A-stage of the immune response during established allergic inflammation. Thus, blockade of the type II receptor should have a beneficial effect on many of the symptoms of asthma and other IL-13R-mediated conditions and should, therefore, be an effective disease modifying agent

Antibodies against IL-13RαI (both monoclonal and polyclonal) have been described in the art; see, eg, WO 97/15663, WO 03/80675; WO 03/46009; WO 06/072564; Gauchat et al, 1998 Eur. J. Immunol. 28:4286-4298; Gauchat et al, 2000 Eur. J. Immunol. 30:3157-3164; Clement et al, 1997 Cytokine 9(11):959 (Meeting Abstract); Ogata et al, 1998 J. Biol. Chem. 273:9864-9871; Graber et al, 1998 Eur. J. Immunol. 28:4286-4298; C. Vermot-Desroches et al, 2000 Tissue Antigens 5(Supp. 1):52-53 (Meeting Abstract); Poudrier et al, 2000 Eur. J. Immunol. 30:3157-3164; Akaiwa et al, 2001 Cytokine 13:75-84; Cancino-Diaz et al, 2002 J. Invest Dermatol. 119:1114-1120; and Krause et al, 2006 Mol. Immunol. 43:1799-1807.

One particularly promising anti-IL-13RαI antibody is CSL334 (previously known as ASLAN004, now called eblasakimab), described in WO2008/060813 as antibody 10G5-6. Eblasakimab has been shown to bind to human IL-13RαI with a high affinity (for example Kd may be 500 pM). ASLAN004 was shown to effectively antagonise IL-13 function through inhibiting the binding of IL-13 to its receptor IL-13RαI and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

Antibodies, such as eblasakimab need to be formulated to high concentration to allow the desired dose in man to be administered in the smallest possible volume. High concentration formulations pose unique challenges as phenomena like phase separation can be observed. Aggregation is also a common feature at high antibody concentration. However, the formulation needs to contain very high levels of antibody molecules as “monomer”, for example 99% monomer or more. In addition, the formulation needs to be stable when stored. Eblasakimab seems to have a hydrophobic portion in the protein, which for example interacts with hydrophobic interaction columns in the absence of high salt concentrations. This hypothesised hydrophobic portion adds additional complexity when formulating the antibody and preventing aggregation. Formation of aggregates and particles in parenteral formulations can be extremely dangerous to patients and must be avoided. Thus, the antibodies of the present disclosure are particularly difficult to formulate. In particular, there is a need to optimise formulations in order to keep viscosity at an acceptably low level for the formulations to be suitable for subcutaneous injection, i.e. close to a target viscosity range of 20-25 cP, such as 20 cP.

Previous work suggested that supplementing formulations with high arginine concentrations of arginine, such as 200 mM or more of arginine-HCl helped to significantly reduce the viscosity of high concentration anti-IL13R antibody formulations. However, for very high concentration formulations with antibody concentrations above 175 mg/ml, such as 200 mg/ml or more, it is especially difficult to keep the viscosity within the target range of 20 to 25 cP.

Thus, optimised formulations are needed to address these problems and/or also to maximise the shelf life, delivery, potency and efficacy of the same.

The present invention employs tryptophan in the formation. Whilst not wishing to be bound by theory the present inventors hypothesis that this amino acid in the formulation can stabilise the hydrophobic portions of the antibody thereby reducing aggregation of antibody molecules.

Thus the present disclosure provides high concentration antibody formulations where the formulated antibody is substantially monomeric and viscosity is acceptable for administration to a patient, such as a human patient

### SUMMARY OF THE DISCLOSURE

The present disclosure is summarised in the following paragraphs:
1. A high concentration formulation of an anti-IL-13R antibody or an antigen binding fragment thereof, said formulation comprising:
   175 to 250 mg/ml of an anti-IL-13R antibody or antigen binding fragment thereof, for example 175, 180, 185, 190, 195, 200, 205, 210, 215, 220, 225, 230, 235, 240, 245 or 250 mg/ml, in particular 190, 200 mg/ml, 210 mg/ml, 225 mg/ml or 250 mg/ml, such as 200 mg/ml of an anti-IL-13R antibody or antigen binding fragment thereof;
   5 to 100 mM of tryptophan (including 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mM), for example 15 to 75 mM of tryptophan, such as 15 to 60 mM, in particular 25 to 50 mM tryptophan, such as 20 mM, 50 or 80 mM tryptophan;
   140 to 290 mM of arginine (including 150 or 151 to 290 nM), for example 160 to 290 mM of arginine (such as Arg-HCl or Arg-Glu), for example 160, 165, 175, 180, 185, 190, 195, 200, 205, 210, 215, 220, 225, 230, 235, 240, 245, 250, 255, 260, 265, 270, 275, 280 285 or 290 mM, in particular 150, 185, 215, 225, 250, 260 or 280 mM arginine;
   0.01-0.03% of a non-ionic surfactant (such as polysorbate), for example 0.01-0.03% w/w, such as 0.01%, 0.015%, 0.020%, 0.025% or 0.030% in particular 0.02% w/w of a non-ionic surfactant;
   a buffer (such as histidine buffer), for example 15 to 55 mM of a buffer (including 25 or 26 to 55), such as 15, 20, 25, 30, 35, 40, 45, 50 or 55 mM of a histidine buffer; in particular 20, 35 or 50 mM histidine buffer; and
   wherein the pH of the formulation is in the range 5.5 to 7.2 (including 5.5, 5.6, 5.7, 5.8, 5.9, 6.0, 6.1, 6.2, 6.3, 6.4, 6.5, 6.6, 6.7, 6.8, 6.9 or 7.0, for example 6.0 to 7.0, such as 6.0, 6.1, 6.2, 6.3, 6.4, 6.5, 6.6, 6.7, 6.8, 6.9 or 7.0, in particular 5.8, 6.3, 6.4, 6.5, 6.6, 6.7 or 6.8.
2. A high concentration formulation of an anti-IL-13R antibody or an antigen binding fragment thereof, said formulation comprising:
   175 to 250 mg/ml of an anti-IL-13R antibody or antigen binding fragment thereof, for example 175, 180, 185, 190, 195, 200, 205, 210, 215, 220 or 225 mg/ml, in particular 200 mg/ml, 225 mg/ml or 250 mg/ml;
   15 to 75 mM of tryptophan, such as 15 to 60 mM, in particular 25 to 50 mM tryptophan; 160 to 290 mM of arginine (such as Arg-HCl or Arg-Glu), for example 160, 165, 175, 180, 185, 190,195, 200, 205, 210, 215, 220, 225, 230, 235, 240, 245, 250, 255, 260, 265, 270, 275, 280 285 or 290 mM, in particular 200, 210, 225, 235, 250 or 260 mM;
   0.01-0.03% of a non-ionic surfactant, for example 0.01-0.03% w/w, such as 0.02% w/w; a buffer (such as histidine buffer); and
   wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.4.
3. A high concentration formulation of an anti-IL-13R antibody or an antigen binding fragment thereof, said formulation comprising:
   175 to 250 mg/ml of an anti-IL-13R antibody or antigen binding fragment thereof, for example 175, 180, 185, 190, 195, 200, 205, 210, 215, 220 or 225 mg/ml, in particular 200 mg/ml, 225 mg/ml or 250 mg/ml;
   15 to 75 mM of tryptophan, such as 15 to 60 mM, in particular 25 to 50 mM tryptophan; 175 to 270 mM of arginine (such as Arg-HCl or Arg-Glu), for example 175, 180, 185, 190, 195, 200, 205, 210, 215, 220, 225, 230, 235, 240, 245, 250, 255, 260, 265 or 270 mM, in particular 200, 210, 225, 235, 250 or 260 mM;
   0.01-0.03% of a non-ionic surfactant, for example 0.01-0.03% w/w, such as 0.02% w/w; a buffer (such as histidine buffer); and
   wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.4.
4. A high concentration formulation of an anti-IL-13R antibody or an antigen binding fragment thereof, said formulation comprising:
   175 to 250 mg/ml of an anti-IL-13R antibody or antigen binding fragment thereof, for example 175, 180, 185, 190, 195, 200, 205, 210, 215, 220 or 225 mg/ml, in particular 200 mg/ml, 225 mg/ml or 250 mg/ml;
   15 to 75 mM of tryptophan, such as 15 to 60 mM, in particular 25 to 50 mM tryptophan; 190 to 270 mM of arginine (such as Arg-HCl or Arg-Glu), for example 190, 195, 200, 205, 210, 215, 220, 225, 230, 235, 240, 245, 250, 255, 260, 265 or 270 mM, in particular 200, 210, 225, 235, 250 or 260 mM;
   0.01-0.03% of a non-ionic surfactant, for example 0.01-0.03% w/w, such as 0.02% w/w; a buffer (such as histidine buffer); and
   wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.4.
5. A high concentration formulation of an anti-IL-13R antibody or an antigen binding fragment thereof, said formulation comprising:
   190 to 210 mg/ml of an anti-IL-13R antibody or antigen binding fragment thereof, for example 190, 195, 200, 205, or 210, in particular 200 mg/ml; 25 to 75 mM of tryptophan, such as 25, 30, 35, 40, 45, 50, 55, 60, 65, 70 or 75 mM tryptophan, in particular 25 to 50 mM tryptophan;
   175 to 270 mM of arginine (such as Arg-HCl or Arg-Glu), for example 190, 195, 200, 205, 210, 215, 220, 225, 230, 235, 240, 245, 250, 255, 260, 265 or 270 mM, in particular 200, 210, 225, 235, 250 or 260 mM;
   0.01-0.03% of a non-ionic surfactant, for example 0.01-0.03% w/w, such as 0.02% w/w; a buffer (such as histidine buffer); and
   wherein the pH of the formulation is in the range 6.3 to 6.7, such as 6.3, 6.4, 6.5, 6.6, 6.7, in particular 6.4, 6.5 or 6.6.
6. A high concentration formulation of an anti-IL-13R antibody or an antigen binding fragment thereof, said formulation comprising:
   190 to 210 mg/ml of an anti-IL-13R antibody or antigen binding fragment thereof, for example 190, 195, 200, 205, or 210, in particular 200 mg/ml;
   25 to 60 mM of tryptophan, such as 25, 30, 35, 40, 45, 50, 55, or 60 mM tryptophan, in particular 25 to 50 mM tryptophan;
   175 to 270 mM of arginine (such as Arg-HCl or Arg-Glu), for example 190, 195, 200, 205, 210, 215, 220, 225, 230, 235, 240, 245, 250, 255, 260, 265 or 270 mM, in particular 200, 210, 225, 235, 250 or 260 mM;
   0.01-0.03% of a non-ionic surfactant, for example 0.01-0.03% w/w, such as 0.02% w/w; a buffer (such as histidine buffer); and
   wherein the pH of the formulation is in the range 6.4 to 6.6, such as 6.4, 6.5 or 6.6.
7. The formulation according to any one of paragraphs 1 to 6, wherein the formulation comprises 190 to 210 mg/ml, such as 190, 195, 200, 205 or 210 mg/ml of the anti-IL13R antibody or a binding fragment thereof.
8. The formulation according to any one of paragraphs 1 to 7, wherein the formulation comprises 200 mg/ml of the anti-IL13R antibody or an antigen binding fragment thereof.
9. The formulation according to any one of paragraphs 1 to 8, wherein the formulation comprises 20 to 80 mM tryptophan.
10. The formulation according to any one of paragraphs 1 to 9, wherein the formulation comprises 15 to 55 mM tryptophan, such as 15, 20, 25, 30, 35, 40, 45, 50 or 55 mM tryptophan.
11. The formulation according to any one of paragraphs 1 to 9, wherein the formulation comprises 25 to 60 mM tryptophan, such as 25, 30, 40, 45, 50, 55 or 60 mM tryptophan.
12. The formulation according to any one of paragraphs 1 to 11, wherein the formulation comprises 20, 50 or 80 mM tryptophan, for example 80 mM tryptophan.
13. The formulation according to any one of paragraphs 1 to 12, wherein the formulation comprises 25 or 50 mM tryptophan.
14. The formulation according to any one of paragraphs 1 to 13, wherein the formulation comprises 50 mM tryptophan.
15. The formulation according to any one of paragraphs 1 to 14, wherein the formulation comprises 150 to 280 mM arginine, such as 150, 155, 160, 165, 170, 175, 180, 185, 190, 195, 200, 205, 210, 215, 220, 225, 230, 235, 240, 245, 250, 255, 260, 265, 270, 275 or 280 mM arginine.
16. The formulation according to any one of paragraphs 1 to 14, wherein the formulation comprises 185 to 260 mM arginine, such as 185, 195, 205, 215, 225, 235, 245, 255 or 260 mM arginine.
17. The formulation according to any one of paragraphs 1 to 16, wherein the formulation comprises 200 to 260 mM arginine, such as 200, 205, 210, 215, 220, 225, 230, 235, 240, 245, 250, 255 or 260 mM arginine.
18. The formulation according to any one of paragraphs 1 to 17, wherein the formulation comprises 150, 185, 215, 225, 260 or 280 mM arginine.
19. The formulation according to any one of paragraphs 1 to 18, wherein the formulation comprises 200, 210, 225, 235, 250 or 260 mM arginine.
20. The formulation according to any one of paragraphs 1 to 19, wherein the formulation comprises 185, 215, 225 or 260 mM arginine.
21. The formulation according to anyone of paragraphs 1 to 20, wherein the arginine is Arg-HCl.
22. The formulation according to any one of paragraphs 1 to 21, wherein the formulation further comprises phenylalanine.
23. The formulation according to paragraph 22, wherein the formulation comprises 25 to 175 mM phenylalanine, such as 25, 30, 35, 40, 45, 50, 55, 60, 65, 70, 75, 80, 85, 90, 95, 100, 105, 110, 115, 120, 125, 130, 135, 140, 145, 150, 155, 160, 165, 170 or 175 mM phenylalanine, such as 50 to 150 mM phenylalanine, for example 50, 55, 60, 65, 70, 75, 80, 85, 90, 95, 100, 105, 110, 115, 120, 125, 130, 135, 140, 145 or 150 mM phenylalanine.
24. The formulation according to paragraphs 22 or 23, wherein the formulation comprises 50, 75, 100, 125 or 150 mM phenylalanine, such as 75, 125 or 150 mM phenylalanine, in particular 150 mM phenylalanine.
25. The formulation according to any one of paragraphs 1 to 24, wherein the formulation further comprises creatine, such as creatine monohydrate.
26. The formulation according to paragraph 25, wherein the formulation comprises 50 to 125 mM creatine monohydrate, for example 50, 55, 60, 65, 70, 75, 80, 85, 90, 95, 100, 105, 110, 115, 120 or 125 mM creatine monohydrate, such as 75 to 100 mM creatine monohydrate, for example 75, 80, 85, 90, 95 or 100 mM creatine monohydrate.
27. The formulation according to paragraph 26, wherein the formulation comprises 75 or 100 mM creatine monohydrate.
28. The formulation according to any one of paragraphs 1 to 27, wherein the formulation comprises 20 to 50 mM histidine buffer, for example 20, 25, 30, 35, 40, 45 or 50 mM, such as 20 mM or 50 mM histidine buffer.
29. The formulation according to any one of paragraphs 1 to 28, wherein the formulation comprises 20 mM histidine buffer.
30. The formulation according to any one of paragraphs 1 to 28, wherein the formulation comprises 50 mM histidine buffer.
31. The formulation according to any one of paragraphs 1 to 30, wherein the formulation further comprises CaCl₂, for example 30, 35, 40, 45, 50, 55, 60, 65 or 70 mM CaCl₂.
32. The formulation according to any one of paragraphs 1 to 31, wherein the formulation comprises 50 mM CaCl₂.
33. The formulation according to any one of paragraphs 1 to 32, wherein the osmolarity of the formulation is in the range 500 to 700 mOsmo/kg, for example 500, 510, 520, 530, 540, 550, 560, 570, 580, 590, 600, 610, 620, 630, 640, 650, 660, 670, 680, 690 or 700 mOsmo/kg, such as 575 to 625 mOsmo/kg, for example 575, 580, 585, 590, 595, 600, 605, 610, 615, 620 or 625 mOsmo/kg.
34. The formulation according to any one of paragraphs 1 to 33, wherein the osmolarity of the formulation is 595 or 600 mOsmo/kg.
35. The formulation according to any one of paragraphs 1 to 34, wherein the formulation further comprises 50 to 200 mM of a sugar, for example 50, 55, 60, 65, 70, 75, 80, 85, 90, 95, 100, 105, 110, 115,120, 125, 130, 135, 140, 145, 150, 155, 160, 165,170, 175, 180, 185, 190, 195, 200, such as 180 mM sugar.
36. The formulation according to any one of paragraphs 1 to 35, wherein the formulation comprises 180 mM sugar.
37. The formulation according to paragraphs 35 or 36, wherein the sugar is selected from mannitol, sorbitol, dextrose, galactose, fructose, lactose, trehalose and sucrose.
38. The formulation according to any one of paragraph 35 to 37, wherein the sugar is sucrose.
39. The formulation according to any one of claims 1 to 38, comprising 0.02% w/w of a non-ionic surfactant.
40. The formulation according to any one of paragraphs 1 to 39, wherein the non-ionic surfactant is polysorbate, for example polysorbate 20, 40, 60, or 80.
41. The formulation according to any one of paragraphs 1 to 40, wherein the non-ionic surfactant is polysorbate 80.
42. The formulation according to any one of paragraphs 1 to 40, wherein the non-ionic surfactant is polysorbate 20.
43. The formulation according to any one of paragraphs 1 to 42, wherein the formulation does not comprise NaCl.
44. The formulation according to any one of paragraphs 1 to 42, wherein the formulation comprises 50 to 150 mM of NaCl, for example 50, 55, 60, 65, 70, 75, 80, 85, 90, 95, 100, 105, 110, 115, 120, 125, 130, 135, 140, 145, 150, such as 62.5 or 140 mM NaCl.
45. The formulation according to any one of paragraphs 1 to 44, wherein the formulation has a viscosity in the range of 10 to 30 cP (mPa·s), for example 10, 11, 12, 13, 14, 15, 16, 17, 18, 19, 20, 21, 22, 23, 24, 25, 26, 27, 28, 29 or 30 cP, such as 15 to 25 cP, in particular 20 cP.
46. The formulation according to any one of paragraphs 1 to 45, wherein the formulation has a viscosity in the range of 15 to 25 cP, for example 15, 20 or 25 cP.
47. The formulation according to any one of paragraphs 1 to 45, wherein the formulation has a viscosity below 25 cP, for example 25, 24, 23, 22, 21 or 20 cP.
48. The formulation according to any one of paragraphs 1 to 47, wherein the formulation has a pH in the range of 5.8 to 6.8, such as 5.8, 5.9, 6.0, 6.1, 6.2, 6.3, 6.4, 6.5, 6.6, 6.7 or 6.8.
49. The formulation according to any one of paragraphs 1 to 48, wherein the formulation has a pH selected from the group comprising 5.8, 6.3, 6.5, 6.7 and 6.8, such as 5.8, 6.7 or 6.8.
50. The formulation according to any one of paragraphs 1 to 47, wherein the formulation has a pH in the range of 6.3 to 6.7, such as 6.3, 6.4, 6.5, 6.6 or 6.7.
51. The formulation according to any one of paragraphs 1 to 47, wherein the formulation has a pH in the range of 6.4 to 6.7, such as 6.4, 6.5, 6.6 or 6.7.
52. The formulation according to any one of paragraphs 1 to 51, comprising or consisting of:
   260 mM of arginine-HCl;
   50 mM tryptophan;
   0.02% of a non-ionic surfactant, such as polysorbate 20; and
   wherein the pH of the formulation is 6.3 to 6.5, such as 6.4.
53. The formulation according to any one of paragraphs 1 to 51, comprising or consisting of:
   225 mM of Arg-HCl;
   75 mM phenylalanine
   50 mM tryptophan;
   0.02% polysorbate 20; and
   wherein the pH of the formulation is 6.3 to 6.5, such as 6.4.
54. The formulation according to any one of paragraphs 1 to 51, comprising or consisting of:
   200 mM of Arg-HCl;
   125 mM phenylalanine
   50 mM tryptophan;
   0.02% polysorbate 20; and
   wherein the pH of the formulation is 6.3 to 6.5, such as 6.4.
55. The formulation according to any one of paragraphs 1 to 51, comprising or consisting of:
   225 mM of Arg-HCl;
   100 mM phenylalanine
   25 mM tryptophan;
   0.02% polysorbate 20; and
   wherein the pH of the formulation is 6.3 to 6.5, such as 6.4.
56. The formulation according to any one of paragraphs 1 to 40, comprising or consisting of:
   260 mM of arginine-HCl;
   50 mM tryptophan;
   0.02% of a non-ionic surfactant, such as polysorbate 20; and
   wherein the pH of the formulation is 6.3 to 6.7, such as 6.4, 6.5 or 6.6.
57. The formulation according to any one of paragraphs 1 to 51, comprising or consisting of:
   225 mM of arginine-HCl;
   75 mM phenylalanine;
   50 mM tryptophan;
   0.02% of a non-ionic surfactant, such as polysorbate 20; and
   wherein the pH of the formulation is 6.3 to 6.7, such as 6.4, 6.5 or 6.6.
58. The formulation according to any one of paragraphs 1 to 51, comprising or consisting of:
   215 mM of arginine-HCl;
   125 mM phenylalanine;
   20 mM tryptophan;
   0.02% of a non-ionic surfactant, such as polysorbate 20; and
   wherein the pH of the formulation is 6.3 to 6.7, such as 6.4, 6.5 or 6.6.
59. The formulation according to any one of paragraphs 1 to 51, comprising or consisting of:
   185 mM of arginine-HCl;
   150 mM phenylalanine;
   50 mM tryptophan;
   0.02% of a non-ionic surfactant, such as polysorbate 20; and
   wherein the pH of the formulation is 6.3 to 6.7, such as 6.4 or 6.5.
60. The formulation according to any one of paragraphs 52 to 59, wherein the formulation comprises 190 to 210 mg/ml, such as 200 mg/ml of an anti-IL13R antibody.
61. The formulation according to any one of paragraphs 1 to 60, wherein the anti-IL-13R antibody or antigen binding fragment thereof is an anti-IL13Rα1 antibody.
62. The formulation according to any one of paragraphs 1 to 61, wherein the anti-IL-13R antibody or antigen binding fragment thereof binds to the epitope FFYQ.
63. The formulation according to any one of paragraphs 1 to 62, wherein the anti-IL-13R antibody or antigen binding fragment thereof comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10.
64. The formulation according to any one of paragraphs 1 to 63, wherein the anti-IL-13R antibody or antigen binding fragment thereof comprises a VH domain comprising an amino acid sequence shown in SEQ ID NO: 51 or a sequence at least 95% identical thereto.
65. The formulation according to any one of paragraphs 1 to 64, wherein the anti-IL-13R antibody or antigen binding fragment thereof comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
66. The formulation according to any one of paragraphs 1 to 65, wherein the anti-IL-13R antibody or antigen binding fragment thereof comprises a VL domain comprising an amino acid sequence shown in SEQ ID NO: 53 or a sequence at least 95% identical thereto.
67. The formulation according to any one of paragraphs 1 to 66, wherein the anti-IL-13R antibody or antigen binding fragment thereof comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10; and comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
68. The formulation according to any one of paragraphs 1 to 67, wherein the anti-IL13R antibody or antigen binding fragment thereof comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto.
69. The formulation according to any one of paragraphs 1 to 68, wherein the anti-IL13R antibody or antigen binding fragment thereof comprises a VH domain comprising an amino acid sequence shown in SEQ ID NO: 51 and a VL domain comprising an amino acid sequence shown in SEQ ID NO: 53.
70. The formulation according to any one of paragraphs 1 to 69, wherein the anti-IL13R antibody or antigen binding fragment thereof is a human antibody.
71. The formulation according to any one of paragraphs 1 to 70, wherein the anti-IL13R antibody is eblasakimab (ASLAN004).
72. The formulation according to any one of paragraphs 1 to 71, wherein the formulation is selected from the group comprising:
   Formulation 1 comprising 200 mg/ml eblasakimab, 20 mM histidine, 150 mM arginine, 80 mM tryptophan, and 0.02% Tween 20, wherein the pH of the formulation is 5.8;
   Formulation 2 comprising 200 mg/ml eblasakimab, 50 mM histidine, 150 mM arginine, 20 mM tryptophan, and 0.02% Tween 20, wherein the pH of the formulation is 6.8;
   Formulation 3 comprising 200 mg/ml eblasakimab, 20 mM histidine, 150 mM arginine, 20 mM tryptophan, and 0.02% Tween 20, wherein the pH of the formulation is 5.8;
   Formulation 4 comprising 200 mg/ml eblasakimab, 50 mM histidine, 280 mM arginine, 20 mM tryptophan, and 0.02% Tween 20, wherein the pH of the formulation is 5.8;
   Formulation 5 comprising 200 mg/ml eblasakimab, 50 mM histidine, 215 mM arginine, 80 mM tryptophan, and 0.02% Tween 20, wherein the pH of the formulation is pH 6.3;
   Formulation 6 comprising 200 mg/ml eblasakimab, 20 mM histidine, 280 mM arginine, 80 mM tryptophan, and 0.02% Tween 20, wherein the pH of the formulation is pH 6.8;
   Formulation 7 comprising 200 mg/ml eblasakimab, 20 mM histidine, 280 mM arginine, 20 mM tryptophan, and 0.02% Tween 20, wherein the pH of the formulation is pH 6.8;
   Formulation 8 comprising 200 mg/ml eblasakimab, 20 mM histidine, 280 mM arginine, 50 mM tryptophan, and 0.02% Tween 20, wherein the pH of the formulation is pH 5.8;
   Formulation 9 comprising 200 mg/ml eblasakimab, 35 mM histidine, 280 mM arginine, 80 mM tryptophan, and 0.02% Tween 20, wherein the pH of the formulation is pH 6.3;
   Formulation 10 comprising 200 mg/ml eblasakimab, 20 mM histidine, 150 mM arginine, 20 mM tryptophan, and 0.02% Tween 20, wherein the pH of the formulation is pH 5.8;
   Formulation 11 comprising 200 mg/ml eblasakimab, 20 mM histidine, 150 mM arginine, 50 mM tryptophan, and 0.02% Tween 20, wherein the pH of the formulation is pH 6.8;
   Formulation 12 comprising 200 mg/ml eblasakimab, 50 mM histidine, 150 mM arginine, 20 mM tryptophan, and 0.02% Tween 20, wherein the pH of the formulation is pH 6.3;
   Formulation 13 comprising 200 mg/ml eblasakimab, 50 mM histidine, 280 mM arginine, 50 mM tryptophan, 0.02% Tween 20, wherein the pH of the formulation is pH 6.8;
   Formulation 14 comprising 200 mg/ml eblasakimab, 20 mM histidine, 150 mM arginine, 80 mM tryptophan, and 0.02% Tween 20, wherein the pH of the formulation is pH 5.8;
   Formulation 15 comprising 200 mg/ml eblasakimab, 20 mM histidine, 150 mM arginine, 80 mM tryptophan, and 0.02% Tween 20, wherein the pH of the formulation is pH 6.3;
   Formulation 16 comprising 200 mg/ml eblasakimab, 35 mM histidine, 150 mM arginine, 80 mM tryptophan, and 0.02% Tween 20, wherein the pH of the formulation is pH 5.8;
   Formulation 17 comprising 200 mg/ml eblasakimab, 20 mM histidine, 215 mM arginine, 20 mM tryptophan, and 0.02% Tween 20, wherein the pH of the formulation is pH 6.8;
   Formulation 18 comprising 200 mg/ml eblasakimab, 50 mM histidine, 280 mM arginine, 80 mM tryptophan, and 0.02% Tween 20, wherein the pH of the formulation is pH 5.8;
   Formulation 19 comprising 200 mg/ml eblasakimab, 20 mM histidine, 260 mM arginine, 50 mM tryptophan, and 0.02% Tween 20, wherein the pH of the formulation is pH 6.3;
   Formulation 20 comprising 200 mg/ml eblasakimab, 20 mM histidine, 260 mM arginine, 50 mM tryptophan, and 0.02% Tween 20, wherein the pH of the formulation is pH 6.8;
   Formulation 21 comprising 200 mg/ml eblasakimab, 50 mM histidine, 150 mM arginine, 80 mM tryptophan, and 0.02% Tween 20, wherein the pH of the formulation is pH 6.8;
   Formulation 22 comprising 200 mg/ml eblasakimab, 20 mM histidine, 280 mM arginine, 20 mM tryptophan, and 0.02% Tween 20, wherein the pH of the formulation is pH 6.8;
   Formulation 23 comprising 200 mg/ml eblasakimab, 50 mM histidine, 150 mM arginine, 50 mM tryptophan, and 0.02% Tween 20, wherein the pH of the formulation is pH 5.8;
   Formulation 24 comprising 200 mg/ml eblasakimab, 35 mM histidine, 280 mM arginine, 20 mM tryptophan, and 0.02% Tween 20, wherein the pH of the formulation is pH 5.8; and
   Formulation 25 comprising 200 mg/ml eblasakimab, 35 mM histidine, 215 mM arginine, 80 mM tryptophan, and 0.02% Tween 20, wherein the pH of the formulation is pH 6.8.
73. The formulation according to any one of paragraphs 1 to 72, wherein the formulation comprises 200 mg/ml eblasakimab, 20 mM histidine, 150 mM arginine, 80 mM tryptophan, and 0.02% Tween 20, and wherein the pH of the formulation is 5.8 (Formulation 1).
74. The formulation according to any one of paragraphs 1 to 72, wherein the formulation comprises 200 mg/ml eblasakimab, 50 mM histidine, 150 mM arginine, 20 mM tryptophan, and 0.02% Tween 20, and wherein the pH of the formulation is 6.8 (Formulation 2).
75. The formulation according to any one of paragraphs 1 to 72, wherein the formulation comprises 200 mg/ml eblasakimab, 20 mM histidine, 150 mM arginine, 20 mM tryptophan, and 0.02% Tween 20, and wherein the pH of the formulation is 5.8 (Formulation 3).
76. The formulation according to any one of paragraphs 1 to 72, wherein the formulation comprises 200 mg/ml eblasakimab, 50 mM histidine, 280 mM arginine, 20 mM tryptophan, and 0.02% Tween 20, and wherein the pH of the formulation is 5.8 (Formulation 4).
77. The formulation according to any one of paragraphs 1 to 72, wherein the formulation comprises 200 mg/ml eblasakimab, 50 mM histidine, 215 mM arginine, 80 mM tryptophan, and 0.02% Tween 20, and wherein the pH of the formulation is pH 6.3 (Formulation 5).
78. The formulation according to any one of paragraphs 1 to 72, wherein the formulation comprises 200 mg/ml eblasakimab, 20 mM histidine, 280 mM arginine, 80 mM tryptophan, and 0.02% Tween 20, and wherein the pH of the formulation is pH 6.8 (Formulation 6).
79. The formulation according to any one of paragraphs 1 to 72, wherein the formulation comprises 200 mg/ml eblasakimab, 20 mM histidine, 280 mM arginine, 20 mM tryptophan, and 0.02% Tween 20, and wherein the pH of the formulation is pH 6.8 (Formulation 7).
80. The formulation according to any one of paragraphs 1 to 72, wherein the formulation comprises 200 mg/ml eblasakimab, 20 mM histidine, 280 mM arginine, 50 mM tryptophan, and 0.02% Tween 20, wherein the pH of the formulation is pH 5.8 (Formulation 8).
81. The formulation according to any one of paragraphs 1 to 72, wherein the formulation comprises 200 mg/ml eblasakimab, 35 mM histidine, 280 mM arginine, 80 mM tryptophan, and 0.02% Tween 20, wherein the pH of the formulation is pH 6.3 (Formulation 9).
82. The formulation according to any one of paragraphs 1 to 72, wherein the formulation comprises 200 mg/ml eblasakimab, 20 mM histidine, 150 mM arginine, 20 mM tryptophan, and 0.02% Tween 20, wherein the pH of the formulation is pH 5.8 (Formulation 10).
83. The formulation according to any one of paragraphs 1 to 72, wherein the formulation comprises 200 mg/ml eblasakimab, 20 mM histidine, 150 mM arginine, 50 mM tryptophan, and 0.02% Tween 20, wherein the pH of the formulation is pH 6.8 (Formulation 11).
84. The formulation according to any one of paragraphs 1 to 72, wherein the formulation comprises 200 mg/ml eblasakimab, 50 mM histidine, 150 mM arginine, 20 mM tryptophan, and 0.02% Tween 20, wherein the pH of the formulation is pH 6.3 (Formulation 12).
85. The formulation according to any one of paragraphs 1 to 72, wherein the formulation comprises 200 mg/ml eblasakimab, 50 mM histidine, 280 mM arginine, 50 mM tryptophan, 0.02% Tween 20, wherein the pH of the formulation is pH 6.8 (Formulation 13).
86. The formulation according to any one of paragraphs 1 to 72, wherein the formulation comprises 200 mg/ml eblasakimab, 20 mM histidine, 150 mM arginine, 80 mM tryptophan, and 0.02% Tween 20, wherein the pH of the formulation is pH 5.8 (Formulation 14).
87. The formulation according to any one of paragraphs 1 to 72, wherein the formulation comprises 200 mg/ml eblasakimab, 20 mM histidine, 150 mM arginine, 80 mM tryptophan, and 0.02% Tween 20, wherein the pH of the formulation is pH 6.3 (Formulation 15).
88. The formulation according to any one of paragraphs 1 to 72, wherein the formulation comprises 200 mg/ml eblasakimab, 35 mM histidine, 150 mM arginine, 80 mM tryptophan, and 0.02% Tween 20, wherein the pH of the formulation is pH 5.8 (Formulation 16).
89. The formulation according to any one of paragraphs 1 to 72, wherein the formulation comprises 200 mg/ml eblasakimab, 20 mM histidine, 215 mM arginine, 20 mM tryptophan, and 0.02% Tween 20, wherein the pH of the formulation is pH 6.8 (Formulation 17).
90. The formulation according to any one of paragraphs 1 to 72, wherein the formulation comprises 200 mg/ml eblasakimab, 50 mM histidine, 280 mM arginine, 80 mM tryptophan, and 0.02% Tween 20, wherein the pH of the formulation is pH 5.8 (Formulation 18).
91. The formulation according to any one of paragraphs 1 to 72, wherein the formulation comprises 200 mg/ml eblasakimab, 20 mM histidine, 260 mM arginine, 50 mM tryptophan, and 0.02% Tween 20, wherein the pH of the formulation is pH 6.3 (Formulation 19).
92. The formulation according to any one of paragraphs 1 to 72, wherein the formulation comprises 200 mg/ml eblasakimab, 20 mM histidine, 260 mM arginine, 50 mM tryptophan, and 0.02% Tween 20, wherein the pH of the formulation is pH 6.8 (Formulation 20).
93. The formulation according to any one of paragraphs 1 to 72, wherein the formulation comprises 200 mg/ml eblasakimab, 50 mM histidine, 150 mM arginine, 80 mM tryptophan, and 0.02% Tween 20, wherein the pH of the formulation is pH 6.8 (Formulation 21).
94. The formulation according to any one of paragraphs 1 to 72, wherein the formulation comprises 200 mg/ml eblasakimab, 20 mM histidine, 280 mM arginine, 20 mM tryptophan, and 0.02% Tween 20, wherein the pH of the formulation is pH 6.8 (Formulation 22).
95. The formulation according to any one of paragraphs 1 to 72, wherein the formulation comprises 200 mg/ml eblasakimab, 50 mM histidine, 150 mM arginine, 50 mM tryptophan, and 0.02% Tween 20, wherein the pH of the formulation is pH 5.8 (Formulation 23).
96. The formulation according to any one of paragraphs 1 to 72, wherein the formulation comprises 200 mg/ml eblasakimab, 35 mM histidine, 280 mM arginine, 20 mM tryptophan, and 0.02% Tween 20, wherein the pH of the formulation is pH 5.8 (Formulation 24).
97. The formulation according to any one of paragraphs 1 to 72, wherein the formulation comprises 200 mg/ml eblasakimab, 35 mM histidine, 215 mM arginine, 80 mM tryptophan, and 0.02% Tween 20, wherein the pH of the formulation is pH 6.8 (Formulation 25).
98. The formulation according to any one of paragraphs 1 to 97, wherein the formulation is stable at refrigerator temperature, for example at 8° C. or less, such as 2 to 8° C., such as 8, 7, 6, 5, 4, 3, 2, or 1° C., in particular 4° C.
99. The formulation according to any one of paragraphs 1 to 98, wherein the formulation is stable at room temperature, for example 15 to 25° C., such as 15, 16, 17, 18, 19, 20, 21, 22, 23, 24 or 25° C., in particular 25° C.
100. The formulation according to any one of paragraphs 1 to 99, wherein the formulation is stable for at least one month, such as at least 2, 3, 6, 12, 18, 24, 30 or 36 months.
101. The formulation according to any one of paragraphs 1 to 100, wherein the formulation is stable at refrigerator temperature, such as 4° C. for at least 6 months.
102. The formulation according to any one of paragraphs 1 to 101, wherein the formulation is stable at room temperature, such as 25° C. for at least 6 months.
103. The formulation according to any one of paragraphs 1 to 102, for use in treatment.
104. The formulation according to paragraph 103, for use in the treatment of inflammation or an autoimmune disease, for example chronic inflammation.
105. The formulation according to paragraph 104, wherein the inflammation is selected from the group comprising: fibrosis (including pulmonary fibrosis, such as cystic fibrosis, idiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease), in particular asthma.
106. The formulation according to any one of paragraphs 1 to 105, for use in the treatment of atopic dermatitis.
107. Use of a formulation according to any one of paragraphs 1 to 102, in the manufacture of a medicament for the treatment of inflammation or an autoimmune disease, for example chronic inflammation.
108. Use of a formulation according paragraph 107 in the manufacture of a medicament for the treatment of a condition selected from the group comprising fibrosis (including pulmonary fibrosis, such as cystic fibrosis, iodiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease), Sezary syndrome, in particular asthma.
109. Use of a formulation according to paragraph 107 or 108, in the manufacture of a medicament for the treatment of atopic dermatitis.
110. A method of treatment comprising administering a therapeutically effective amount of a formulation according to any one of paragraphs 1 to 102.
111. A method of treating inflammation (such as chronic inflammation) or an autoimmune disease comprising administering an effective amount of a formulation according to any one of paragraphs 1 to 102.
112. The method according to paragraph 111, wherein the treatment of a condition selected from the group comprising: group comprising: fibrosis (including pulmonary fibrosis, such as cystic fibrosis, idiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease), in particular asthma.
113. The method according to any one of paragraphs 110 to 112, wherein the method is for the treatment of atopic dermatitis.

In one embodiment the formulation of the present disclosure comprises arginine, phenylalanine and tryptophan.

In one embodiment the patient is a human.

In one embodiment the formulations of the present disclosure are physically stable, for example no/minimal aggregation.

In one embodiment the formulations of the present disclosure are chemically stable, for example no/minimal deamination.

In one embodiment the formulations of the present disclosure are thermally stable.

The present inventors have optimised the formulation of the present disclosure and established that the IL-13R antibodies, such as eblasakimab, are most suitable for formulation within a narrow set of parameters. The formulations of the present disclosure are highly monomeric, for example at least 98% monomeric (such as 98 to 99.5% monomeric) even when formulated with high antibody concentration. The formulations of the present disclosure also have good viscosity at high antibody concentrations.

The presently disclosed anti-IL13R antibody formulation is particularly suitable for stable long-term storage of the anti-IL13R antibody. In one embodiment the formulation is stored at a temperature in the range 2 to 8° C., such as 2, 3, 4, 5, 6, 7 or 8° C., such as 4° C.

In addition, the formulation is suitably stable, for example in some embodiments no change in monomer or less than a 0.5% reduction in monomer was observed when stored at 4° C. or 25° C. for 90 days. In comparison, Dupixent® (dupilumab) can only be stored at 25° C. for a maximum of 14 days. Accelerated ‘stress test’ studies at 40° C. also show the formulations of the present disclosure to be stable over a period of 60 days, for example using potency measurements.

In one embodiment certain formulations of the present disclosure have 1% or less protein aggregation, for example when stored for 90 days at temperature in the range 2 to 25° C. In one embodiment, the formulations of the present disclosure have 1% or less protein aggregation when stored for 90 days at 4° C. In one embodiment, the formulations of the present disclosure have 1% or less protein aggregation when stored for 90 days at 25° C.

Hence, in one embodiment, the formulations of the present disclosure are stable at refrigerator temperature, for example at 8° C. or less, such as 2 to 8° C., such as 8, 7, 6, 5, 4, 3, 2, or 1° C., in particular 4° C. In another embodiment, the formulations of the present disclosure are stable at room temperature, for example 15 to 25° C., such as 15, 16, 17, 18, 19, 20, 21, 22, 23, 24 or 25° C., in particular 25° C. Thus, in one embodiment, the formulations are stable at 4° C. In another embodiment, the formulations are stable at 25° C.

In one embodiment, the formulations of the present disclosure are stable for at least one month, such as 28, 29, 30 or 31 days. In one embodiment, the formulations of the present disclosure are stable for at least 2 months or at least 60 days. In one embodiment, the formulations are stable for at least 3 months or at least 90 days. In one embodiment, the formulations are stable for at least 6 months. In one embodiment, the formulations are stable for at least 12 months. In one embodiment, the formulations are stable for at least 18 months. In one embodiment, the formulations are stable for at least 24 months. In one embodiment, the formulations are stable for at least 30 months. In one embodiment, the formulations are stable for at least 36 months.

In one embodiment, the formulations of the present disclose are stable at room temperature, for example 15 to 25° C., such as 15, 16, 17, 18, 19, 20, 21, 22, 23, 24 or 25° C., in particular 25° C. for at least for at least one month, such as 28, 29, 30 or 31 days. In one embodiment, the formulations of the present disclosure are stable at room temperature, for example 15 to 25° C., such as 15, 16, 17, 18, 19, 20, 21, 22, 23, 24 or 25° C., in particular 25° C. for at least 60 days. In one embodiment, the formulations are stable at room temperature, for example 15 to 25° C., such as 15, 16, 17, 18, 19, 20, 21, 22, 23, 24 or 25° C., in particular 25° C. for at least 90 days. In one embodiment, the formulations are stable at room temperature, for example 15 to 25° C., such as 15, 16, 17, 18, 19, 20, 21, 22, 23, 24 or 25° C., in particular 25° C. for at least 6 months. In one embodiment, the formulations are stable at room temperature, for example 15 to 25° C., such as 15, 16, 17, 18, 19, 20, 21, 22, 23, 24 or 25° C., in particular 25° C. for at least 12 months. In one embodiment, the formulations are stable at room temperature, for example 15 to 25° C., such as 15, 16, 17, 18, 19, 20, 21, 22, 23, 24 or 25° C., in particular 25° C. for at least 18 months. In one embodiment, the formulations are stable at room temperature, for example 15 to 25° C., such as 15, 16, 17, 18, 19, 20, 21, 22, 23, 24 or 25° C., in particular 25° C. for at least 24 months. In one embodiment, the formulations are stable fat room temperature, for example 15 to 25° C., such as 15, 16, 17, 18, 19, 20, 21, 22, 23, 24 or 25° C., in particular 25° C. or at least 30 months. In one embodiment, the formulations are stable at room temperature, for example 15 to 25° C., such as 15, 16, 17, 18, 19, 20, 21, 22, 23, 24 or 25° C., in particular 25° C. for at least 36 months.

In one embodiment, the formulations of the present disclosure are stable at refrigerator temperature, for example at 8° C. or less, such as 2 to 8° C., such as 8, 7, 6, 5, 4, 3, 2, or 1° C., in particular 4° C., for at least one month, such as 28, 29, 30 or 31 days. In one embodiment, the formulations of the present disclosure are stable at refrigerator temperature, for example at 8° C. or less, such as 2 to 8° C., such as 8, 7, 6, 5, 4, 3, 2, or 1° C., in particular 4° C., for at least 60 days. In one embodiment, the formulations are stable at refrigerator temperature, for example at 8° C. or 30 less, such as 2 to 8° C., such as 8, 7, 6, 5, 4, 3, 2, or 1° C., in particular 4° C., for at least 90 days. In one embodiment, the formulations are stable at refrigerator temperature, for example at 8° C. or less, such as 2 to 8° C., such as 8, 7, 6, 5, 4, 3, 2, or 1° C., in particular 4° C., for at least 6 months. In one embodiment, the formulations are stable at refrigerator temperature, for example at 8° C. or less, such as 2 to 8° C., such as 8, 7, 6, 5, 4, 3, 2, or 1° C., in particular 4° C., for at least 12 months. In one 35 embodiment, the formulations are stable at refrigerator temperature, for example at 8° C. or less, such as 2 to 8° C., such as 8, 7, 6, 5, 4, 3, 2, or 1° C., in particular 4° C., for at least 18 months. In one embodiment, the formulations are stable for at least 24 months. In one embodiment, the formulations are stable at refrigerator temperature, for example at 8° C. or less, such as 2 to 8° C., such as 8, 7, 6, 5, 4, 3, 2, or 1° C., in particular 4° C., for at least 30 months. In one embodiment, the 40 formulations are stable at refrigerator temperature, for example at 8° C. or less, such as 2 to 8° C., such as 8, 7, 6, 5, 4, 3, 2, or 1° C., in particular 4° C., for at least 36 months.

The combination of features of the formulation of the present disclosure, including the pH, contribute to stabilising the IL-13 receptor antibody or binding fragment thereof.

In particular, the present inventors have established that the addition of tryptophan to high concentration anti-13R antibody formulations resulted in a dramatic reduction in viscosity 5 allowing very high concentration formulations comprising 200 mg/ml or more anti-IL13R antibody to achieve viscosities within the target range of 20 to 25 cP, such as 20 cP.

In one embodiment the formulations of the present disclosure have a viscosity in the range of 10 to 30, such as 11, 12, 13, 14, 15, 16, 17, 18, 19, 20, 21, 22, 23, 24, 25, 26, 27, 28, 29 or 30 cP (centipoise), such as 20 cP, for example at ambient temperature. In one embodiment, the formulations have a viscosity in the range 10 to 25 cP, such as 10, 11, 12, 13, 14, 15, 16, 17, 18, 19, 20, 21, 22, 23, 24 or 25 cP. In one embodiment, the formulations have a viscosity in the range 15 to 25, such as 15, 16, 17, 18, 19, 20, 21, 22, 23, 24 or 25 cP. In one embodiment, the formulations have a viscosity in the range 20 to 25 cP, such as 20, 21, 22, 23, 24 or 25 cP. In one embodiment, the formulations have a viscosity below 25 cP, such as 25, 24, 23, 22, 21, 20, 19, 18, 17, 16 or 15 cP. Surprisingly, the viscosity of the formations of the present disclosure are relatively low even at high concentrations of antibody.

In one embodiment, the viscosity is measured using a viscometer, such as rotational viscometer, an electromagnetically spinning-sphere (EMS) viscometer, or a Stabinger viscometer. In one embodiment the viscosity is measured using a rheometer, such as shear rheometer, dynamic shear rheometer, an extensional rheometer, a capillary rheometer. In one embodiment the viscosity is measured using a Kinexus-ultra+ rheometer (Netzsch).

In one embodiment the osmolarity of the formulation is in the range 510 to 575 mOsmo/kg, such as 520 to 570 mOsmo/kg, such as 520, 525, 530, 535, 540, 545, 550, 555, 560, 565, 570 or 575 mOsmo/kg. In one embodiment, the osmolarity of the formulation is in the range 500 to 600 mOsmo/kg, such as 500, 505, 510, 515, 520, 525, 530, 535, 540, 545, 550, 555, 560, 565, 570, 575, 580, 585, 590, 595 or 600 mOsmo/kg. In one embodiment, the osmolarity of the formulation is in the range 550 to 600, such as 550, 555, 560, 565, 570, 575, 580, 585, 590, 595 or 600 mOsmo/kg.

In one embodiment the formulation comprises 175 to 250 mg/ml, such as 175, 180, 185, 190, 195, 200, 205, 210, 215, 220, 225, 230, 235, 240, 245 or 250 mg/ml of an anti-IL13Rα1 antibody or an antigen binding fragment thereof, for example 175 to 225 mg/ml, such as 175, 180, 185, 190, 195, 200, 205, 210, 215, 220 or 225 mg/ml of an anti-IL13Rα1 antibody or an antigen binding fragment thereof. In one embodiment, the formulation comprises 200 mg/ml or more, such as 200, 205, 210, 215, 220 or 225 mg of an anti-IL13Rα1 antibody or an antigen binding fragment thereof. In one embodiment, the formulation comprises 190 mg/ml to 210 mg/ml, such as 190, 195, 200, 205 or 210 mg/ml of an anti-IL13Rα1 antibody or an antigen binding fragment thereof. In one embodiment the formulation comprises about 200 mg/ml of anti-IL13Rα1 antibody or an antigen binding fragment thereof, such as 200 mg/ml of anti-IL13Rα1 antibody. In one embodiment, the formulation comprises 195 mg/ml of an anti-IL13Rα1 antibody or antigen binding fragment thereof. In one embodiment, the formulation comprises 190 mg/ml of an anti-IL13Rα1 antibody or antigen binding fragment thereof.

In one embodiment the anti-IL13Rα1 antibody or binding fragment thereof comprises a variable heavy region comprising a CDRH1 with a sequence shown in SEQ ID NO: 1, a CDRH2 with a sequence shown in SEQ ID NO: 3, and a CDRH3 with a sequence shown in SEQ ID NO: 30; and a variable light region comprising CDRL1 with a sequence shown in SEQ ID NO: 31, a CDRL2 with a sequence shown in SEQ ID NO: 32, and a CDRL3 with a sequence shown in SEQ ID NO: 33.

In one embodiment, the anti-IL13Rα1 antibody or binding fragment thereof comprises a VH domain with a sequence shown in SEQ ID NO: 51 or a sequence at least 95% identical thereto. In one embodiment the antibody or binding fragment thereof comprises a VL domain with a sequence shown in SEQ ID NO: 53 or a sequence at least 95% identical thereto.

In one embodiment, the anti-IL13Rα1 antibody or binding fragment thereof comprises a VH domain with a sequence shown in SEQ ID NO: 51 or a sequence at least 95% identical thereto and a VL domain with a sequence shown in SEQ ID NO: 53 or a sequence at least 95% identical thereto. In one embodiment, the anti-IL13Rα1 antibody or binding fragment thereof comprises a VH domain with a sequence shown in SEQ ID NO: 51 and a VL domain with a sequence shown in SEQ ID NO: 53.

In one embodiment, the anti-IL13Rα1 antibody or binding fragment thereof is eblasakimab (ASLAN004).

In one embodiment, the formulation comprises 100 mM tryptophan or less, such as 100, 95, 90, 85, 80, 75, 70, 65, 60, 55 or 50 mM tryptophan. In one embodiment, the formulation comprises 90 mM tryptophan or less, such as 90, 85, 80, 75, 70, 65, 60, 55 or 50 mM tryptophan. In one embodiment, the formulation comprises 80 mM tryptophan or less, such as 80, 75, 70, 65, 60, 55 or 50 mM tryptophan. In one embodiment, the formulation comprises 70 mM tryptophan or less, such as 70, 65, 60, 55 or 50 mM tryptophan. In one embodiment, the formulation comprises 60 mM tryptophan or less, such as 60, 55, 50, 45, 40, 35, 30, 25 or 20 mM tryptophan. In one embodiment, the formulation comprises 50 mM tryptophan or less, such as 50, 45, 40, 35, 30, 25 or 20 mM tryptophan.

Formulations of the present disclosure contain a minimum level of 5 mM of tryptophan.

In one embodiment, the formulation comprises 5 to 100 mM tryptophan, such as 5, 10, 15, 20, 25, 30, 35, 40, 45, 50, 55, 60, 65, 70, 75, 80, 85, 90, 95 or 100 mM tryptophan. In one embodiment, the formulation comprises 10 to 90 mM tryptophan, such as 10, 15, 20, 25, 30, 35, 40, 45, 50, 55, 60, 65, 70, 75, 80, 85 or 90 mM tryptophan. In one embodiment, the formulation comprises 15 to 85 mM tryptophan, such as 15, 20, 25, 30, 35, 40, 45, 50, 55, 60, 65, 70, 75, 80, 85 mM tryptophan. In one embodiment, the formulation comprises 20 to 80 mM tryptophan, such as 20, 25, 30, 35, 40, 45, 50, 55, 60, 65, 70, 75 or 80 mM tryptophan. In one embodiment, the formulation comprises 25 to 75 mM tryptophan, such as 25, 30, 35, 40, 45, 50, 55, 60, 65, 70 or 75 mM tryptophan. In one embodiment, the formulation comprises 20 to 70 mM tryptophan, such as 20, 25, 30, 35, 40, 45, 50, 55, 60, 65, 70, 75 or 80 mM tryptophan. In one embodiment, the formulation comprises 20 to 60 mM tryptophan, such as 20, 25, 30, 35, 40, 45, 50, 55 or 60 mM tryptophan.

In one embodiment, the formulation comprises 20 to 55 mM tryptophan, such as 20, 25, 30, 35, 40, 45, 50 or 55 mM tryptophan. In one embodiment, the formulation comprises 25 to 50 mM tryptophan, for example 25, 30, 35, 40, 45 or 50 mM tryptophan. In one embodiment, the formulation comprises 20, 25, 50 or 80 mM tryptophan. In one embodiment, the formulation comprises 20 mM tryptophan. one embodiment, the formulation comprises 25 mM tryptophan. In another embodiment, the formulation comprises 50 mM tryptophan. In one embodiment, the formulation comprises 80 mM tryptophan.

The tryptophan in the formulations of the present invention may be employed as salt, such as a potassium or sodium salt.

In one embodiment the tryptophan is NOT employed as a salt.

In one embodiment tryptophan is L-tryptophan.

In one embodiment, the formulation comprises 290 mM or less arginine, such as 290, 285, 280, 275, 270, 265, 260, 255, 250, 245, 240, 235, 230, 225, 220, 215, 210, 205 or 200 mM arginine. In one embodiment, the formulation comprises 280 mM or less arginine, such as 280, 275, 270, 265, 260, 255, 250, 245, 240, 235, 230, 225, 220, 215, 210, 205 or 200 mM arginine. In one embodiment, the formulation comprises 270 mM or less arginine, such as 270, 265, 260, 255, 250, 245, 240, 235, 230, 225, 220, 215, 210, 205 or 200 mM arginine. In one embodiment, the formulation comprises 260 mM or less arginine, such as 260, 255, 250, 245, 240, 235, 230, 225, 220, 215, 210, 205 or 200 mM arginine.

Formulations of the present disclosure contain at least 140 mM of arginine.

In one embodiment, the formulation comprises 140 to 290 mM arginine, such as 140, 145, 150, 155, 160, 165, 170, 175, 180, 185, 190, 195, 200, 205, 210, 215, 220, 225, 230, 240, 245, 250, 255, 260, 265, 270, 275, 280, 285 or 290 mM arginine. In one embodiment, the formulation comprises 150 to 280 nM arginine, such as 150, 155, 160, 165, 170, 175, 180, 185, 190, 195, 200, 205, 210, 215, 220, 225, 230, 235, 240, 245, 250, 255, 260, 265, 270, 275 or 280 nM arginine. In one embodiment, the formulation comprises 160 to 270 nM arginine, such as 160, 165, 170, 175, 180, 185, 190, 195, 200, 205, 210, 215, 220, 225, 230, 235, 240, 245, 250, 255, 260, 265 or 270 nM arginine. In on embodiment, the formulation comprises 175 to 265 nM arginine, such as 175, 180, 185, 190, 195, 200, 205, 210, 215, 220, 225, 230, 235, 240, 245, 250, 255, 260 or 265 nM arginine.

In one embodiment, the formulation comprises 185 to 260 mM arginine, such as arginine-HCl, such as 185, 195, 205, 215, 225, 235, 245, 255 or 260 mM arginine. In one embodiment, the formulation comprises 190 to 250 nM arginine, such as 190, 195, 200, 205, 210, 215, 220, 225, 230, 235, 240, 245 or 250 nM arginine. In one embodiment, the formulation comprises 200 to 260 mM of arginine, for example 200, 205, 210, 215, 220, 225, 230, 235, 240, 245, 250 or 260 mM. In one embodiment, the formulation comprises 175 to 195 mM arginine, such as 175, 180, 185, 190 or 195 mM. In one embodiment the formulation comprises 250 to 270 mM arginine, such as 250, 255, 260, 265 or 270 mM arginine.

In one embodiment, the formulation comprises 150, 215, 260 or 280 mM arginine. In one embodiment, the formulation comprises 200, 210, 225, 235, 250 or 260 mM arginine. In one embodiment, the formulation comprises 185 mM arginine. In one embodiment, the formulation comprises 150 mM arginine. In one embodiment, the formulation comprises 200 mM arginine. In one embodiment, the formulation comprises 210 mM arginine. In one embodiment, the formulation comprises 215 mM arginine. In one embodiment, the formulation comprises 225 mM arginine. In one embodiment, the formulation comprises 235 mM arginine. In one embodiment, the formulation comprises 250 mM arginine. In one embodiment, the formulation comprises 260 mM arginine. In one embodiment, the formulation comprises 280 mM arginine.

In one embodiment, the arginine is Arg-HCl. In another embodiment, the arginine is Arg-Glu. In one embodiment arginine is L-arginine. Hence, in one embodiment the formulation comprises 185 to 260 mM Arg-HCl. In one embodiment the formulation comprises 200 to 260 mM Arg-HCl. In one embodiment, the formulation comprises 200, 210, 225, 235, 250 or 260 mM Arg-HCl. In one embodiment, the formulation comprises 185 mM Arg-HCl. In one embodiment, the formulation comprises 200 mM Arg-HCl. In one embodiment, the formulation comprises 210 mM Arg-HCl. In one embodiment, the formulation comprises 215 mM Arg-HCl. In one embodiment, the formulation comprises 225 mM Arg-HCl. In one embodiment, the formulation comprises 235 mM 20 Arg-HCl. In one embodiment, the formulation comprises 250 mM Arg-HCl. In one embodiment, the formulation comprises 260 mM Arg-HCl.

In one embodiment the formulation comprises 0.01-0.03% of a non-ionic surfactant, such as 0.01, 0.015, 0.02, 0.025 or 0.030%, in particular 0.02%. In one embodiment the formulation comprises 0.01-0.03%, such as 0.01, 0.015, 0.02, 0.025 or 0.030%, in particular 0.02% volume per volume (v/v) of a non-ionic surfactant. In one embodiment the formulation comprises 0.01-0.03%, such as 0.01, 0.015, 0.02, 0.025 or 0.030%, in particular 0.02% weight per volume (w/v) of a non-ionic surfactant. In one embodiment the formulation comprises 0.01-0.03%, such as 0.01, 0.015, 0.02, 0.025 or 0.030%, in particular 0.02% weight per weight (w/w) of a non-ionic surfactant. In one embodiment the formulation comprises 0.02% w/w of a non-ionic surfactant.

In one embodiment the non-ionic surfactant is polysorbate, such as polysorbate 20, 40, 60, or 80, such as 20 or 80. In one embodiment the non-ionic surfactant is polysorbate 80. Thus, in one embodiment the formulation comprises 0.01-0.03%, such as 0.02% polysorbate 80 (for example as % w/w, % w/v, % v/w or % v/v). In one embodiment the formulation comprises 0.02% w/w polysorbate 80. In one embodiment the non-ionic surfactant is polysorbate 20 (Tween 20). Thus, in one embodiment the formulation comprises 0.01-0.03%, such as 0.010%, 0.015%, 0.020%, 0.025% or 0.030% polysorbate 20 (for example as % w/w, % w/v, % v/w or % v/v). In one embodiment, the formulation comprises 0.02% w/w polysorbate 20.

In one embodiment wherein the pH of the formulation is in the range 5.5 to 7.2, such as 5.5, 5.6, 5.7, 5.8, 5.9, 6.0, 6.1, 6.2, 6.3, 6.4, 6.5, 6.6, 6.7, 6.8, 6.9, 7.0, 7.1 or 7.2. In one embodiment, the pH of the formulation is in the range 5.7 to 7.0, such as 5.7, 5.8, 5.9, 6.0, 6.1, 6.2, 6.3, 6.4, 6.5, 6.6, 6.7, 6.8, 6.9 or 7.0. In one embodiment, the pH is in the range 5.8 to 6.8, such as 5.9, 6.0, 6.1, 6.2, 6.3, 6.4, 6.5, 6.6, 6.7 or 6.8. In one embodiment wherein the pH of the formulation is in the range 6.0 to 7.0, such as 6.0, 6.1, 6.2, 6.3, 6.4, 6.5, 6.6, 6.7, 6.8, 6.9 or 7.0. In one embodiment, the pH of the formulation is in the range 6.2 to 6.8, such as 6.2, 6.3, 6.4, 6.5, 6.6, 6.7 or 6.8.

In one embodiment, the pH is 6.8 or less, such as 6.8, 6.7, 6.6, 6.5, 6.4, 6.3, 6.2, 6.1 or 6.0. In one embodiment, the pH is 6.7 or less, such as 6.7, 6.6, 6.5, 6.4, 6.3, 6.1, 6.1 or 6.0. In one embodiment, the pH is 6.6 or less, such as 6.6, 6.5, 6.4, 6.3, 6.1, 6.1 or 6.0. In one embodiment, the pH is 6.5 or less, such as 6.5, 6.4, 6.3, 6.1, 6.1 or 6.0. In one embodiment, the pH is in the range 6.3 to 6.7, such as 6.3, 6.4, 6.5, 6.6 or 6.7. In one embodiment the pH is 6.3 to 6.5, such as 6.3, 6.4, or 6.5, for example 6.4. In one embodiment the pH is 6.4 to 6.6, such as 6.4, 6.5 or 6.6. In one embodiment, the pH is 6.5 to 6.7, such as 6.5, 6.6 or 6.7. In one embodiment, the pH is 5.8, 6.3, 6.5, 6.6, 6.7 or 6.8. In one embodiment, the pH is 5.8. In one embodiment, the pH is 6.3. In one embodiment, the pH is 6.4. In one embodiment, the pH is 6.5. In one embodiment, the pH is 6.6. In one embodiment, the pH is 6.7. In one embodiment, the pH is 6.8.

In one embodiment, the formulation further comprises phenylalanine, such as 45 to 90 mM phenylalanine, for example 45, 50, 55, 60, 65, 70, 75, 80, 85 or 90 mM. In one embodiment, the formulation further comprises wherein the formulation comprises 25 to 175 mM phenylalanine, such as 50 to 150 mM phenylalanine, for example 25, 30, 35, 40, 45, 50, 55, 60, 65, 70, 75, 80, 85, 90, 95, 100, 103, 110, 115, 120, 125, 130, 135, 140, 145 or 150 mM phenylalanine. In one embodiment the formulation comprises 65 to 160 mM phenylalanine, for example 65, 70, 75, 80, 85, 90, 95, 100, 105, 110, 115, 120, 125, 130, 135, 140, 145, 150, 155 or 160 mM. In one embodiment, the formulation comprises 75 to 150 mM phenylalanine, such as 75, 80, 85, 90, 95, 100, 105, 110, 115, 120, 125, 130, 135, 140, 145, 150. In one embodiment, the formulation comprises 115 to 160 mM phenylalanine, such as 125 to 150 mM phenylalanine, for example 125, 130, 135, 140, 145 or 150 mM phenylalanine. In one embodiment, the formulation comprises 50, 75, 100, 125 or 150 mM phenylalanine, such as 75, 125 or 150 mM phenylalanine.

In one embodiment the formulation comprises 50, 75 or 80 mM phenylalanine. In one embodiment the formulation comprises 50 mM phenylalanine. In one embodiment, the formulation comprises 75 mM phenylalanine. In one embodiment the formulation comprises 80 mM phenylalanine. In one embodiment the formulation comprises 125 mM phenylalanine. In one embodiment the formulation comprises 150 mM phenylalanine.

In one embodiment, the formulation further comprises creatine, such as creatine monohydrate, creatine ethyl ester, creatine hydrochloride, buffered creatine or creatine magnesium chelate. In one embodiment, the formulation comprises creatine monohydrate. In one embodiment, the formulation comprises 50 to 125 mM creatine, for example 50, 55, 60, 65, 70, 75, 80, 85, 90, 95, 100, 105, 110, 115, 120 or 125 mM creatine. In one embodiment, the formulation comprises 75 to 100 mM creatine, such as 75 to 100 mM creatine monohydrate. In one embodiment, the formulation comprises 75, 80, 85, 90, 95, or 100 creatine, such as 75, 80, 85, 90, 95, or 100 creatine monohydrate. In one embodiment, the formulation comprises 75 mM creatine monohydrate. In one embodiment, the formulation comprises 100 mM creatine monohydrate.

In one embodiment, the formulation comprises 15 to 55 mM histidine buffer, such as 15, 20, 25, 30, 35, 40, 45, 50 or 55 mM histidine buffer. In one embodiment the formulation comprises 20 to 50 mM histidine buffer, for example 20, 25, 30, 35, 40, 45 or 50 mM, such as 20 mM or 50 mM histidine buffer. In one embodiment, the formulation comprises 25 to 50 mM histidine buffer, such as 25, 30, 35, 40, 45 or 50 mM histidine buffer. In one embodiment, the formulation comprises 35 to 50 mM histidine buffer, such as 35, 40, 45 or 50 mM histidine buffer. In one embodiment, the formulation comprises 20, 35 or 50 mM histidine buffer. In one embodiment the formulation comprises 20 mM histidine buffer. In one embodiment, the formulation comprises 35 mM histidine buffer. In another embodiment the formulation comprises 50 mM histidine buffer.

In one embodiment, the formulation does not comprise a phosphate buffer, such as sodium or potassium phosphate buffer.

In one embodiment the formulation further comprises CaCl₂, for example 10, 20, 30, 40, 50 or 60 mM CaCl₂. In one embodiment the formulation comprises 50 mM CaCl₂.

In one embodiment the formulation does NOT comprise CaCl₂.

In one embodiment the formulation of the present disclosure does NOT comprise NaCl and/or KCl.

In one embodiment the formulation further comprises 50 to 200 mM of a sugar, such as 50, 60, 70, 80, 90, 100, 110, 120, 130, 140, 150, 160, 170, 180, 190 or 200 mM of a sugar. In one embodiment the formulation comprises 180 mM of a sugar. In one embodiment the sugar is selected from mannitol, sorbitol, dextrose, galactose, fructose, lactose, trehalose and sucrose. In one embodiment the sugar is sucrose. Thus, in one embodiment the formulation comprises 180 mM sucrose.

In one embodiment the formulation of the present disclosure does NOT comprise a sugar, in particular a sugar disclosed herein, such as sucrose.

In one embodiment there is provided a parenteral formulation (in particular a liquid formulation) for example for infusion or injection. In one embodiment there is provided liquid parenteral formulation as a concentrate for dilution with a liquid for injection, such as glucose, saline or water for injection. In one embodiment the liquid parenteral formulation is provided in a final concentration for administration without dilution, for example for injection or for infusion.

In one embodiment the antibody or binding fragment employed in the formulation of the present disclosure is monoclonal.

In one embodiment the antibody or binding fragment employed in the formulation of the present disclosure is human. In one embodiment the antibody or binding fragment employed in the formulation of the present disclosure is chimeric or humanised.

In one embodiment the formulation is NOT F2 from FIG. 1. In one embodiment the formulation is NOT F4 from FIG. 1. In one embodiment the formulation is NOT F7 from FIG. 1. In one embodiment the formulation is NOT F9 from FIG. 1. In one embodiment the formulation is NOT F10 from FIG. 1.

In one embodiment, the formulation comprises:
190 to 210 mg/ml of an anti-IL-13R antibody or antigen binding fragment thereof, for example 190, 195, 200, 205, or 210, in particular 200 mg/ml;
40 to 60 mM of tryptophan, such as 40, 45, 50, 55, or 60 tryptophan, in particular 50 mM tryptophan;
175 to 270 mM of arginine (such as Arg-HCl or Arg-Glu), for example 190, 195, 200, 205, 210, 215, 220, 225, 230, 235, 240, 245, 250, 255, 260, 265 or 270 mM, in particular 185, 215, 225, or 260 200, 260 mM;
0.01-0.03% of a non-ionic surfactant, for example 0.01-0.03% w/w, such as 0.02% w/w; a buffer (such as histidine buffer); and
wherein the pH of the formulation is in the range 6.3 to 6.7, such as 6.3, 6.4, 6.5, 6.6, 6.7, in particular 6.4, 6.5 or 6.6.

In one embodiment, the formulation comprises:
190 to 210 mg/ml of an anti-IL-13R antibody or antigen binding fragment thereof, for example 190, 195, 200, 205, or 210, in particular 200 mg/ml;
40 to 60 mM of tryptophan, such as 40, 45, 50, 55, or 60 tryptophan, in particular 50 mM tryptophan;
250 to 270 mM of arginine (such as Arg-HCl or Arg-Glu), for example 250, 255, 260, 265 or 270 mM, in particular 260 mM;
0.01-0.03% of a non-ionic surfactant, for example 0.01-0.03% w/w, such as 0.02% w/w; a buffer (such as histidine buffer); and
wherein the pH of the formulation is in the range 6.3 to 6.7, such as 6.3, 6.4, 6.5, 6.6, 6.7, in particular 6.4, 6.5 or 6.6.

In one embodiment, the formulation comprises:
190 to 210 mg/ml of an anti-IL-13R antibody or antigen binding fragment thereof, for example 190, 195, 200, 205, or 210, in particular 200 mg/ml;
40 to 60 mM of tryptophan, such as 40, 45, 50, 55, or 60 tryptophan, in particular 50 mM tryptophan;
175 to 195 mM of arginine (such as Arg-HCl or Arg-Glu), for example 175, 180, 185, 190 or 195 mM, in particular 185 mM;
0.01-0.03% of a non-ionic surfactant, for example 0.01-0.03% w/w, such as 0.02% w/w; a buffer (such as histidine buffer); and
wherein the pH of the formulation is in the range 6.3 to 6.7, such as 6.3, 6.4, 6.5, 6.6, 6.7, in particular 6.4, 6.5 or 6.6.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 260 mM of arginine-HCl; 50 mM tryptophan; 0.02% of a non-ionic surfactant; and wherein the pH of the formulation is 6.4. In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 225 mM of Arg-HCl; 75 mM phenylalanine; 50 mM tryptophan; 0.02% polysorbate 20; and wherein the pH of the formulation is 6.4.

In one embodiment, the formulation comprises: 190 to 210 mg/ml, such as 200 mg/ml of an 30 anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 200 mM of Arg-HCl; 125 mM phenylalanine; 50 mM tryptophan; 0.02% polysorbate 20; and wherein the pH of the formulation is 6.4.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 225 mM of Arg-HCl; 100 mM phenylalanine; 25 mM tryptophan; 0.02% polysorbate 20; and wherein the pH of the formulation is 6.4.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 260 mM of arginine-HCl; 50 mM tryptophan; 0.02% of a non-ionic surfactant; and wherein the pH of the formulation is 6.3 to 6.7, such as 6.4 or 6.5.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 225 mM of arginine-HCl; 75 mM phenylalanine; 50 mM tryptophan; 0.02% of a non-ionic surfactant; and wherein the pH of the formulation is 6.3 to 6.7, such as 6.4 or 6.5.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 215 mM of arginine-HCl; 125 mM phenylalanine; 20 mM tryptophan; 0.02% of a non-ionic surfactant; and wherein the pH of the formulation is 6.3 to 6.7, such as 6.4 or 6.5.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 185 mM of arginine-HCl; 150 mM phenylalanine; 50 mM tryptophan; 0.02% of a non-ionic surfactant; and wherein the pH of the formulation is 6.3 to 6.7, such as 6.4 or 6.5.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 260 mM of arginine-HCl; 50 mM tryptophan; 0.02% of polysorbate 20; and wherein the pH of the formulation is 6.3 to 6.7, such as 6.4, 6.5 or 6.6.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 225 mM of arginine-HCl; 75 mM phenylalanine; 50 mM tryptophan; 0.02% of polysorbate 20; and wherein the pH of the formulation is 6.3 to 6.7, such as 6.4, 6.5 or 6.6.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 215 mM of arginine-HCl; 125 mM phenylalanine; 20 mM tryptophan; 0.02% of polysorbate 20; and wherein the pH of the formulation is 6.3 to 6.7, such as 6.4, 6.5 or 6.6.

In one embodiment, the formulation comprises: 190 to 210 mg/ml, such as 200 mg/ml of an anti-IL13R antibody or an antigen binding fragment thereof, wherein the anti-IL13R antibody comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto; 185 mM of arginine-HCl; 150 mM phenylalanine; 50 mM tryptophan; 0.02% of polysorbate 20; and wherein the pH of the formulation is 6.3 to 6.7, such as 6.4, 6.5 or 6.6.

In one embodiment the formulation is manufactured and/or filled under nitrogen.

In one embodiment the formulation is filled in opaque vials, for example amber vials that block light.

### DETAILED DISCLOSURE

Long term as used herein refers to a period of at least 6 months, such as 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 months. In one embodiment, the disclosed formulation storage for at least 12 months, such as 12 months, 18 months and 24 months.

Non-ionic surfactant as employed herein refers to surfactants that have covalently bonded oxygen-containing hydrophilic groups which are bonded to hydrophobic parent structures. Examples of non-ionic surfactants include ethoxylates, such as fatty alcohol ethoxylates (such as narrow-range ethoxylate, octaethylene glycol monododecyl ether and pentaethylene glycol monododecyl ether), alkylphenol ethoxylates (such as nonoxynols and Triton X-100), fatty acid ethoxylates, ethoxylated amines and/or fatty acid amides (such as polyethoxylated tallow amine, cocamide monoethanolamine and cocamide diethanolamine), terminally blocked ethoxylates (such as poloxamers); fatty acid esters of polyhydroxy compounds; fatty acid esters of glycerol (such as glycerol monostearate and glycerol monolaurate); fatty acid esters of sorbitol (such as sorbitan monolaurate, sorbitant monosterate and sorbitan tristearate; Tweens such as Tween 20, 40, 60 or 80); fatty acid esters of sucrose; alkyl polyglucosides (such as decyl glucoside, lauryl glucoside and octyl glucose); and polysorbates (such as polysorbate 20, 40, 60 or 80).

Thus, in one embodiment the non-ionic surfactant is selected from the group comprising ethoxylates; fatty acid esters of polyhydroxy compounds; fatty acid esters of glycerol; fatty acid esters of sorbitol; Tweens; fatty acid esters of sucrose; alkyl polyglucosides; and polysorbates.

Parenteral formulation as employed herein refers to a formulation designed not to be delivered through the GI tract Typical parenteral delivery routes include injection (including bolus injection), implantation or infusion. In one embodiment the formulation is provided in a form for bolus delivery.

In one embodiment the parenteral formulation is administered intravenously. In one embodiment the parenteral formulation is administered subcutaneously.

Injection as employed herein refers to the administration of a liquid formulation into the body via a syringe or syringe driver. Injection includes intravenous, subcutaneous or intramuscular administration. The injection is generally over a short period of time, such as 5 minutes or less. However, injection can be administered slowly or continuously, for example using a syringe driver. Injections generally involve administration of smaller volumes than infusions. In one embodiment the injection is administered as a slow injection, for example over a period of 1.5 to minutes. Slow injection as employed herein is manual injection with syringe. In one embodiment one dose of the formulation less than 100 mls, for example 30 mls, such as administered by a syringe driver. In one embodiment, a dose of the anti-IL13R antibody or antigen binding fragment thereof, such as eblasakimab is 100 to 600 mg, such as 100 mg, 200 mg, 300 mg, 400, 500 mg or 600 mg. In one embodiment, the dose is 300 mg. In one embodiment, the dose is 400 mg. In one embodiment, the dose is 500 mg. In one embodiment, the dose is 600 mg.

Advantageously, the high concentration formulation of the present disclosure comprises 175 to 250 mg/ml, such as 190 to 210 mg/ml, in particular 200 mg/ml of an anti-IL-13R antibody or antigen binding fragment thereof. Subcutaneous injections are typically limited to a maximum volume of 2 ml. Thus, the present high concentration formulation allows for high dosages, such as 400 or 500 mg to be administered via a single subcutaneous injection.

Hence, in one embodiment, the dose is administered via subcutaneous injection, such as via multiple or single subcutaneous injections. In one embodiment, the dose is administered via a single subcutaneous injection. In one embodiment, one dose of the formulation is less than 4 ml, for example 4 ml, 3.5 ml, 3.0 ml, 2.5 ml, 2.0 ml, 1.5 ml, 1 ml or 0.5 ml. In one embodiment, one dose of the formulation is 2 ml or less, such as 2.0 ml, 1.5 ml or 0.5 ml. In one embodiment, one dose of the formulation is 2 ml. In one embodiment, one dose of the formulation is 1.5 ml. In one embodiment, one dose of the formulation is 1.0 ml. In one embodiment, one dose of the formulation is 0.5 ml.

In one embodiment, a dose of 500 mg is administered via a single subcutaneous injection. In one embodiment, a dose of 400 mg is administered via a single subcutaneous injection. In one embodiment, a dose of 300 mg is administered via a single subcutaneous injection. In one embodiment, a dose of 200 mg is administered via a single subcutaneous injection. In one embodiment, a dose of 100 mg is administered via a single subcutaneous injection.

In one embodiment, a dose of 400 mg is administered via a single 2 ml subcutaneous injection.

Infusion as employed herein means the administration of fluids by drip, infusion pump, or equivalent device. In one embodiment the infusion is administered over a period in the range of 1 to 120 minutes (for example 1 to 5 minutes), such as about 1, 3, 4, 5, 6, 7, 8, 9, 10, 11, 12, 13, 14, 15, 16, 17, 18, 1920, 25, 30, 35, 40, 45, 50, 55, 60, 65, 70, 65, 80, 85, 90, 95, 100, 105, 110, 115 or 120 minutes.

### Anti-IL13R Antibody

Interleukin-13 receptor (IL-13R) as used herein is a type I cytokine receptor, which binds to Interleukin-13. It consists of two subunits, encoded by IL13Rα1 and IL4R, respectively. These two genes encode the proteins IL-13Rα1 and IL-4Rα. These form a dimer with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STAT6. Human IL-13Rα1 has the Uniprot number P3597.

IL-13Rα2, previously called IL-13R and IL-13Rα, is another receptor which is able to bind to IL-13. However, in contrast to IL-13Rα1, this protein binds IL-13 with high affinity, but it does not bind IL-4. Human IL-13Rα2 has the Uniprot number Q14627.

Anti-IL13R antibody as used herein refers to an antibody that has specificity for IL13R, for example IL13Rα1 or IL13Rα2. In one embodiment, the anti-IL13R antibody of the present disclosure is specific for IL13Rα1. In one embodiment, the anti-IL13R antibody binds to an epitope comprising the amino acid sequence FFYQ.

The anti-IL13R antibodies of the present disclosure may comprise a complete antibody molecule having full length heavy and light chains or a binding fragment thereof. Binding fragments include but are not limited to Fab, modified Fab, Fab′, F(ab′)₂, Fv, single domain antibodies (such as VH, VL, VHH, IgNAR V domains), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217).

The methods for creating and manufacturing these antibody fragments are well known in the art (see for example Verma et al, 1998, Journal of Immunological Methods, 216, 165-181). Other antibody fragments for use in the present invention include the Fab and Fab′ fragments described in WO2005/003169, WO2005/003170 and WO2005/003171. Other antibody fragments for use in the present invention include Fab-Fv and Fab-dsFv fragments described in WO2010/035012 and antibody fragments comprising those fragments. Multi-valent antibodies may comprise multiple specificities or may be monospecific (see for example WO 92/22853 and WO05/113605).

The antibody and fragments thereof, for use in the present disclosure may be from any species including for example mouse, rat, shark, rabbit, pig, hamster, camel, llama, goat or human. Chimeric antibodies have a non-human variable regions and human constant regions.

An antibody or binding fragment for use in the present invention can be derived from any class (e.g. IgG, IgE, IgM, IgD or IgA) or subclass of immunoglobulin molecule. In one embodiment the antibody employed in the present disclosure is IgG4 or IgG4 with a 241 P mutation.

In one embodiment the antibody or binding fragment employed in the formulation of the present disclosure has affinity of 5 nM or higher (higher affinity is a lower numerical value), for example 500 pM, such as 250 pM or higher, in particular 125 pM or less.

In one embodiment CDRH1 is an amino acid sequence GYSFTSYWIG (SEQ ID NO: 1).

In one embodiment CDRH2 is an amino acid sequence VIYPGDSYTR (SEQ ID NO: 2).

In one embodiment CDRH3 has the formula:

SEQ ID NO: 3
X₁ Pro Asn Trp Gly X₆ X₇ Asp X₉

X₁ denotes Phe, Met, Gln, Leu or Val
X₆ denotes Ser or Ala
X₇ denotes Phe, Leu, Ala or Met
X₉ denotes Tyr, Gln, Lys, Arg, Trp, His, Ala, Thr,
Ser, Asn or Gly

In one embodiment the IL13-R1α1 antibody or binding fragment employed in the formulation of the present disclosure comprises a CDRH3 in dependently selected from SEQ ID NO: 4 to 30.

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: or 3.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH3 having the amino acid sequence MPNWGSLDH (SEQ ID NO: 10)

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

In one embodiment CDRL1 is an amino acid sequence RASQSISSSYLA SEQ ID NO: 31.

In one embodiment CDRL2 is an amino acid sequence GASSRAT SEQ ID NO: 32.

In one embodiment CDL3 has the formula:

SEQ ID NO: 33
Gln X₂X₃X₄Xₛ

X₂ denotes Gln, Arg, Met, Ser, Thr or Val.
X₃ denotes Tyr or Val.
X₄ denotes Glu, Ala, Gly or Ser.
Xₛ denotes Thr, Ala or Ser.

In one embodiment the IL-13Rα1 antibody employed in the formulation of the present disclosure comprises a CDRL3 in dependently selected from SEQ ID NO: 34 to 47.

In one embodiment, the anti-IL13R antibody or binding fragment employed in the present disclosure comprises a CDL3 having the amino acid sequence QQYAS (SEQ ID NO: 45).

In one embodiment, the anti-IL13R antibody or binding fragment employed in the present disclosure comprises a CDRL1 comprising an amino acid sequence as set forth in SEQ ID NO: 31, a CDRL2 comprising an amino acid sequence as set forth in SEQ ID NO: 32, and a CDRL3 comprising an amino acid sequence as set forth in SEQ ID NO: 45.

In one embodiment the IL-13Rα1 antibody employed in the formulation of the present disclosure comprises a CDRL3 independently selected from a sequence comprising SEQ ID NO: 34 to 47.

In one embodiment CDRL3 comprising an amino acid sequence as set forth in SEQ ID NO: 33.

In one embodiment the VH region is independently selected from a sequence from the group comprising: SEQ ID NO: 48; SEQ ID NO: 49; SEQ ID NO: 50; SEQ ID NO: 51 and a sequence at least 95% identical to any one of the same.

SEQ ID NO: 51
Glu Val Gln Leu Val Gln Ser Gly Ala Glu Val Lys Lys Pro Gly Glu Ser Leu Lys Ile Ser Cys Lys
Gly Ser Gly Tyr Ser Phe Thr Ser Tyr Trp Ile Gly Trp Val Arg Gln Met Pro Gly Lys Gly Leu Glu
Trp Met Gly Val Ile Tyr Pro Gly Asp Ser Tyr Thr Arg Tyr Ser Pro Ser Phe Gln Gly Gln Val Thr
Ile Ser Ala Asp Lys Ser Ile Ser Thr Ala Tyr Leu Gln Trp Ser Ser Leu Lys Ala Ser Asp Thr Ala
Met Tyr Tyr Cys Ala Arg Met Pro Asn Trp Gly Ser Leu Asp His Trp Gly Gln Gly Thr Leu Val Thr
Val Ser Ser

In one embodiment the VL is independently selected from a sequence from the group comprising: SEQ ID NO: 52, SEQ ID NO: 53 and SEQ ID NO: 54 and a sequence at least 95% identical to any one of the same (* K deleted in a post translational modification).

SEQ ID NO: 53
EIVLTQSPGTLSLSPGERATLSCRASQSISSSYLAWYQQKPGQAPRLLIYGASSRATGIP
DRFSGSGSGTDFTLTISRLEPEDFAVYYCQQYASFGQGTKVEI*

In one embodiment the VH sequence is SEQ ID NO: 48 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VH sequence is SEQ ID NO: 49 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VH sequence is SEQ ID NO: 50 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VH sequence is SEQ ID NO: 51 (or a sequence at least 95% identical thereto) and the VL sequence is SEQ ID NO: 52, SEQ ID NO: 53 or SEQ ID NO: 54 (or a sequence at least 95% identical to any one of the same). In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51. (or a sequence at least 95% identical to any one of the same). In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same). In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same). In one embodiment the VH sequence is SEQ ID NO: 51 (or a sequence at least 95% identical thereto) and the VL sequence is SEQ ID NO: 53 (or a sequence at least 95% identical thereto).

Variable region as employed herein refers to the region in an antibody chain comprising the CDRs and a suitable framework.

In one embodiment the heavy chain comprises a sequence independently selected from the group comprising: SEQ ID NO: 55, 56, 57, 58, 59, 60 and a sequence at least 95% identical to any one of the same (*K deleted in a post translational modification).

In one embodiment the light chain is independently selected from a group comprising: SEQ ID NO: 61: SEQ ID NO: 62; SEQ ID NO: 63 and a sequence at least 95% identical to any one of the same.

In one embodiment the heavy chain is independently selected from SEQ ID NO: 55, 56, 57, 58, 59 and 60 (or a sequence at least 95% identical to any one of the same) and the light chain is independently selected from SEQ ID NO: 61 62 and 63 (or a sequence at least 95% identical to any one of the same).

In one embodiment the heavy chain is SEQ ID NO: 55 (or a sequence at least 95% identical thereto) and the light chain is independently selected from SEQ ID NO: 61 or 62 and 63 (or a sequence at least 95% identical to any one of the same). In one embodiment the heavy chain is SEQ ID NO: 56 (or a sequence at least 95% identical thereto) and the light chain is independently selected from SEQ ID NO: 61 or 62 and 63 (or a sequence at least 95% identical to any one of the same). In one embodiment the heavy chain is SEQ ID NO: 57 (or a sequence at least 95% identical thereto) and the light chain is independently selected from SEQ ID NO: 62 or 63 and 63 (or a sequence at least 95% identical to any one of the same). In one embodiment the heavy chain is SEQ ID NO: 58 (or a sequence at least 95% identical thereto) and the light chain is independently selected from SEQ ID NO: 61 62 and 63 (or a sequence at least 95% identical to any one of the same). In one embodiment the heavy chain is SEQ ID NO: 59 (or a sequence at least 95% identical thereto) and the light chain is independently selected from SEQ ID NO: 61 62 and 63 (or a sequence at least 95% identical to any one of the same). In one embodiment the heavy chain is SEQ ID NO: 60 (or a sequence at least 95% identical thereto) and the light chain is independently selected from SEQ ID NO: 61 61 and 63 (or a sequence at least 95% identical to any one of the same). In one embodiment the heavy chain is SEQ ID NO: 58 or 60 (or a sequence at least 95% identical to any one of the same) and a light chain with the sequence shown in SEQ ID NO: 61 (or a sequence at least 95% identical thereto). In one embodiment the heavy chain is SEQ ID NO: 58 (or a sequence at least 95% identical to any one of the same) and a light chain with the sequence shown in SEQ ID NO: 61 (or a sequence at least 95% identical thereto). In one embodiment the heavy chain is SEQ ID NO: 60 (or a sequence at least 95% identical to any one of the same) and a light chain with the sequence shown in SEQ ID NO: 61 (or a sequence at least 95% identical thereto).

In one embodiment, the anti-IL13R antibody is eblasakimab (previously known as ASLAN004).

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human.

Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP 0438474 and EP0463151.

Constant region as employed herein is intended to refer to the constant region portion located between two variable domains, for example non-cognate variable domains, in the heavy chain. Thus, the presently disclosed anti-IL13R antibody may comprise one or more constant regions, such as a naturally occurring constant domain or a derivate of a naturally occurring domain.

A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO03031581. Alternatively, where the effector molecule is a protein or polypeptide the linkage may be achieved using recombinant DNA procedures, for example as described in WO 86/01533 and EP0392745.

The term effector molecule as used herein includes, for example, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof. Specific naturally occurring polymers include lactose, amylase, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In one embodiment, the presently disclosed formulation is suitable for use in the treatment of a chronic inflammatory condition. Examples of chronic inflammatory conditions include but are not limited to atopic dermatitis, asthma, rheumatoid arthritis, psoriatic arthritis, lupus, inflammatory bowel disease such as ulcerative colitis and Crohn's disease, diabetes such as type 1 or type diabetes, ankylosing spondylitis, gout., myositis, scleroderma, Sjogren's syndrome, endometriosis and vasculitis. Hence, in one embodiment, the patient to be treated has a chronic inflammatory condition selected from the group comprising atopic dermatitis, asthma, rheumatoid arthritis, psoriatic arthritis, lupus, inflammatory bowel disease such as ulcerative colitis and Crohn's disease, diabetes such as type 1 or type 2 diabetes, ankylosing spondylitis, gout, myositis, scleroderma, Sjogren's syndrome, endometriosis and vasculitis.

In one embodiment, the formulation is for use in the treatment of atopic dermatitis, such as moderate to severe atopic dermatitis. In one embodiment, the formulation is for use in the treatment of pruritus.

In patients with cancer, such as breast cancer, cancer related lymphedema (BCRL), the formulation of the present disclosure may prevent lymphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lymphedema after lymphatic injury is associated with tissue inflammation, the infiltration of CD4-positive cells and their differentiation to the type 2 helper T-cell (Th2) phenotype. Th2 cells produce IL-4 and IL-13 that play a key role in the development of lymphedema-associated symptoms as well as other Th2-mediated diseases.

In one embodiment the formulation herein is administered in combination with another therapy.

“In combination” as employed herein is intended to encompass where the anti-IL13R antibody is administered before, concurrently with another therapy.

Therapeutic dose as employed herein refers to the amount of the anti-IL13R antibody, such as eblasakimab (ASLAN004) that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment a formulation according to the present disclosure (including a formulation comprising same) is administered monthly, for example in a treatment cycle or as maintenance therapy.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

Subject headings herein are employed to divide the document into sections and are not intended to be used to construe the meaning of the disclosure provided herein.

The application claims priority from U.S. 63/373,566 filed on 26 Aug. 2022, SG10202260455R filed on 15 Dec. 2022 and SG10202301 153T filed on 25 Apr. 2023, all of which are incorporated herein by reference, and any of which may be employed to correct errors in the present specification.

The background contains technical information and can be used as basis for amendments.

Individual values in the examples may be extracted and combined with generic elements of the disclosure i.e. can be used as specific basis for amendment without reference to the other features of the example.

The present invention is further described by way of illustration only the following examples.

### BRIEF SUMMARY OF THE FIGURES

FIG. 1 Table showing the composition of the formulations tested, including theoretical osmolality.

FIG. 2 Flow chart showing experimental design, including summary of how the formulations were prepared

FIG. 3 Photographs of formulations for the purpose of visual inspection

FIG. 4 Table showing measured pH, osmolality and protein concentration of formulations tested

FIG. 5 Table showing viscosity measurements of formulations tested. F7-R and F8-R are repeat measurements of F7 and F8 respectively.

FIG. 6 Graph showing viscosity (at room temperature) of formulations tested

FIG. 7 Table showing the composition of formulations tested in round 2 screen

FIG. 8 Flow chart showing experimental design, including summary of how the formulations were prepared for formulations in round 2 screen

FIG. 9 Photograph of formulations from round 2 screen for purpose of visual inspection

FIG. 10 Table showing pH, osmolality and protein concentration of formulations from round 2 screen. Stock protein concentration was measured by three analysts with 16 individual 2× diluted protein stock solution by Solo VPE.

The measured protein stock concentration ranges from 190-210 mg/mL and an average of 203±7 mg/mL with 95% confidence level. In Round 1 the average protein concentration was slightly lower at 200 mg/mL.

FIG. 11 Table showing viscosity measurements of formulations from round 2 screen

FIG. 12 Graph showing viscosity results of formulations from round 2 screen

FIG. 13 Table showing the compositions of the formulations from round 2 screen that were selected for stability studies. Formulation 3 was tested at both 195 mg/ml and 200 mg/ml target concentrations

FIG. 14A Photographs of formulations at T=0 for the purpose of visual inspection. Color reference according to Ph Eur (Sigma Aldrich 86293-1SET-F)

FIG. 14B Photographs of formulations at T=4 weeks @4° C. for the purpose of visual inspection. Color reference according to Ph Eur (Sigma Aldrich 86293-1SET-F)

FIG. 14C Photographs of formulations at T=4 weeks @25° C. for the purpose of visual inspection. Color reference according to Ph Eur (Sigma Aldrich 86293-1SET-F)

FIG. 14D Photographs of formulations at T=4 weeks @40° C. for the purpose of visual inspection. Color reference according to Ph Eur (Sigma Aldrich 86293-1SET-F)

FIG. 15A Table summarising visual inspection results for formulations at T=0 weeks. Color reference according to Ph Eur (Sigma Aldrich 86293-1SET-F)

FIG. 15B Table summarising visual inspection results for formulations at T=4 weeks@4° C. Color reference according to Ph Eur (Sigma Aldrich 86293-1SET-F)

FIG. 15C Table summarising visual inspection results for formulations at T=4 weeks@25° C. Color reference according to Ph Eur (Sigma Aldrich 86293-1SET-F)

FIG. 15D Table summarising visual inspection results for formulations at T=4 weeks @40° C. Color reference according to Ph Eur (Sigma Aldrich 86293-1 SET-F)

FIG. 16 Photographs showing colour comparison between current formulations vs previous eblasakimab formulations (WP3B)

FIG. 17 Table showing pH of formulations at T=4 weeks vs T=0 weeks

FIG. 18 Table showing concentration by reverse phased high-performance liquid chromatography (RP-HPLC) of formulations at T=4 weeks vs T=0 weeks. *Concentrations of T=0, agitation and 5× F/T were adjusted by correcting the concentration of stock reference standard solution that was used for standard curve from 100 mg/mL to 105.2 mg/mL. The concentration stock reference standard solution (BDS from JHL) was measured by SoloVPE to be 105.2 mg/mL instead of 100 mg/mL

FIG. 19 Viscosity curve (tangential flow filtration (TFF) diluted samples) for formulations with increasing eblaskimab concentration. JHL formulation=previous eblasakimab formulation with good viscosity up to 100 mg/ml concentration

FIG. 20 Graph and table showing viscosity results of formulations at T=4 weeks vs T=0. *Concentration of F@ is ~212 mg/ml

FIG. 21A Graph and table showing size exclusion-high-performance liquid chromatography (SE-HPLC) results for formulations at T=0

FIG. 21B Graph and table showing SE-HPLC results for formulations at T=4 weeks @4° C.

FIG. 21C Graph and table showing SE-HPLC results for formulations at T=4 weeks @25° C.

FIG. 21D Graph and table showing SE-HPLC results for formulations at T=4 weeks @40° C.

FIG. 22 Graphs summarising SE-HPLC results—% monomer and % soluble aggregates for formulations at T=4 weeks vs T=0

FIG. 23 Graphs summarising physical stability of presently claimed formulations vs previous eblasakimab formulations (WP3B) based on SE-HPLC results. *F3-195 at 195 mg/mL

FIG. 24A Graph and table showing cation exchange-high-performance liquid chromatography (CEX-HPLC) results for formulations at T=0

FIG. 24B Graph and table showing CEX-HPLC results for formulations at T=4 weeks @4° C.

FIG. 24C Graph and table showing CEX-HPLC results for formulations at T=4 weeks @25° C.

FIG. 24D Graph and table showing CEX-HPLC results for formulations at T=4 weeks @40° C.

FIG. 25 Graphs summarising chemical stability of presently claimed formulations based on CEX-HPLC results.

FIG. 26A Graph showing results from micro-flow imaging (MFI) analysis for particle size range >5 μM—indicator of physical stability and quality. All samples were diluted to 20 mg/ml in the corresponding formulation buffer for MFI analysis. MFI particle counts were based on average of two measurements.

FIG. 26B Graph showing results from non-spherical (non-SPH) analysis for particle size range >5 μM—indicator of physical stability and quality. All samples were diluted to 20 mg/ml in the corresponding formulation buffer for MFI analysis. MFI particle counts were based on average of two measurements. Non-spherical analysis: aspect ratio <0.75

FIG. 27A Graphs comparing MFI and non-SPH analysis results for present formulations with previous eblasakimab formulations (WP3B) for size range >10 μM

FIG. 27B Graphs comparing MFI and non-SPH analysis results for present formulations with previous eblasakimab formulations (WP3B) for size range >25 μM

FIG. 28A Photographs of formulations at T=8 weeks @4° C. for the purpose of visual inspection. Color reference according to Ph Eur (Sigma Aldrich 86293-1SET-F)

FIG. 28B Photographs of formulations at T=8 weeks @25° C. for the purpose of visual inspection. Color reference according to Ph Eur (Sigma Aldrich 86293-1SET-F)

FIG. 28C Photographs of formulations at T=8 weeks @40° C. for the purpose of visual inspection. Color reference according to Ph Eur (Sigma Aldrich 86293-1SET-F)

FIG. 29A Table summarising visual inspection results for formulations at T=8 weeks @4° C. TNTC=too numerous to count. Color reference according to Ph Eur (Sigma Aldrich 86293-1SET-F)

FIG. 29B Table summarising visual inspection results for formulations at T=8 weeks @25° C. TNTC=too numerous to count. Color reference according to Ph Eur (Sigma Aldrich 86293-1SET-F)

FIG. 29C Table summarising visual inspection results for formulations at T=8 weeks @40° C. Color reference according to Ph Eur (Sigma Aldrich 86293-1 SET-F)

FIG. 30 Table showing pH of formulations at T=8 weeks vs T=4 weeks and T=0

FIG. 31 Table showing concentration by RP-HPLC of formulations at T=8 weeks vs T=4 weeks and T=0. *Concentrations of T=0, agitation and 5× F/T were corrected by correcting the concentration of stock reference standard solution that used for standard curve from 100 mg/mL to 105.2 mg/mL. The concentration stock reference standard solution were measured by SoloVPE to be 105.2 mg/mL instead of 100 mg/mL

FIG. 32 Table showing osmolality (vapor pressure) of formulations at T=8 weeks vs T=4 weeks and T=0

FIG. 33 Graph and table showing viscosity results of formulations at T=8 weeks vs T=4 weeks and T=0

FIG. 34A Graph and table showing SE-HPLC results for formulations at T=8 weeks @4° C.

FIG. 34B Graph and table showing SE-HPLC results for formulations at T=8 weeks @25° C.

FIG. 35A Graphs summarising SE-HPLC results for formulations at T=8 weeks @4° C.

FIG. 35B Graphs summarising SE-HPLC results for formulations at T=8 weeks @25° C.

FIG. 36A Graph and table showing CEX-HPLC results for formulations at T=8 weeks @4° C.

FIG. 36B Graph and table showing CEX-HPLC results for formulations at T=8 weeks @25° C.

FIG. 37A Graphs summarising CEX-HPLC results for formulations at T=8 weeks @4° C.

FIG. 37B Graphs summarising CEX-HPLC results for formulations at T=8 weeks @25° C.

FIG. 38A Graphs showing MFI and non-SPH analysis results for formulations at T=8 weeks vs T=4 weeks and T=0 for >5 μM

FIG. 38B Graphs showing MFI and non-SPH analysis results for formulations at T=8 weeks vs T=4 weeks and T=0 for >10 μM

FIG. 38C Graphs showing MFI and non-SPH analysis results for formulations at T=8 weeks vs T=4 weeks and T=0 for >25 μM

FIG. 39A Photographs of formulations at T=26 weeks @4° C. for the purpose of visual inspection. Color reference according to Ph Eur (Sigma Aldrich 86293-1SET-F)

FIG. 39B Photographs of formulations at T=26 weeks @25° C. for the purpose of visual inspection. Color reference according to Ph Eur (Sigma Aldrich 86293-1SET-F)

FIG. 40A Table summarising visual inspection results for formulations at T=26 weeks @4° C. TNTC=too numerous to count. Color reference according to Ph Eur (Sigma Aldrich 86293-1SET-F)

FIG. 40B Table summarising visual inspection results for formulations at T=26 weeks @25° C. TNTC=too numerous to count Color reference according to Ph Eur (Sigma Aldrich 86293-1SET-F)

FIG. 41 Table showing pH of formulations at T=26 weeks, T=8 weeks, T=4 weeks and T=0

FIG. 42 Table showing concentration by RP-HPLC of formulations at T=26 weeks, T=8 weeks, T=4 weeks and T=0. *Concentrations of T=0, agitation and 5× F/T were corrected by correcting the concentration of stock reference standard solution that used for standard curve from 100 mg/mL to 105.2 mg/mL. The concentration stock reference standard solution were measured by SoloVPE to be 105.2 mg/mL instead of 100 mg/mL

FIG. 43 Table showing osmolality (vapor pressure) of formulations at T=26 weeks, T=8 weeks, T=4 weeks and T=0

FIG. 44 Graph and table showing viscosity results of formulations at T=26 weeks, T=8 weeks, T=4 weeks and T=0

FIG. 45A Graph and table showing SE-HPLC results for formulations at T=26 weeks @4° C.

FIG. 45B Graph and table showing SE-HPLC results for formulations at T=26 weeks @25° C.

FIG. 46A Graphs summarising SE-HPLC results for formulations at T=26 weeks @4° C.

FIG. 46B Graphs summarising SE-HPLC results for formulations at T=26 weeks @25° C.

FIG. 47A Graph and table showing CEX-HPLC results for formulations at T=26 weeks @4° C.

FIG. 47B Graph and table showing CEX-HPLC results for formulations at T=26 weeks @25° C.

FIG. 48A Graphs summarising CEX-HPLC results for formulations at T=26 weeks @4° C.

FIG. 48B Graphs summarising CEX-HPLC results for formulations at T=26 weeks @25° C.

FIG. 49A Table showing MFI analysis results for formulations at T=26 weeks @4° C.

All samples were diluted to 20 mg/mL in the corresponding formulation buffer for MFI analysis. MFI particle counts: average of two measurements non-Spherical: aspect ratio <0.75

FIG. 49B Table showing MFI analysis results for formulations at T=26 weeks @25° C. All samples were diluted to 20 mg/mL in the corresponding formulation buffer for MFI analysis. MFI particle counts: average of two measurements non-Spherical: aspect ratio <0.75

FIG. 50A Graphs showing MFI and non-SPH analysis results for formulations at T=26 weeks, T=8 weeks, T=4 weeks and T=0 weeks for >5 μM. All samples were diluted to 20 mg/mL in the corresponding formulation buffer for MFI analysis. MFI particle counts: average of two measurements non-Spherical: aspect ratio <0.75

FIG. 50B Graphs showing MFI and non-SPH analysis results for formulations at T=26 weeks, T=8 weeks, T=4 weeks and T=0 weeks for >25 μM. All samples were diluted to 20 mg/mL in the corresponding formulation buffer for MFI analysis. MFI particle counts: average of two measurements non-Spherical: aspect ratio <0.75

### EXAMPLES

### Example 1—Production and Testing of High Concentration 200 mg/ml Anti-IL13R Eblasakimab (ASLAN004) Formulations—Round 1 Screen

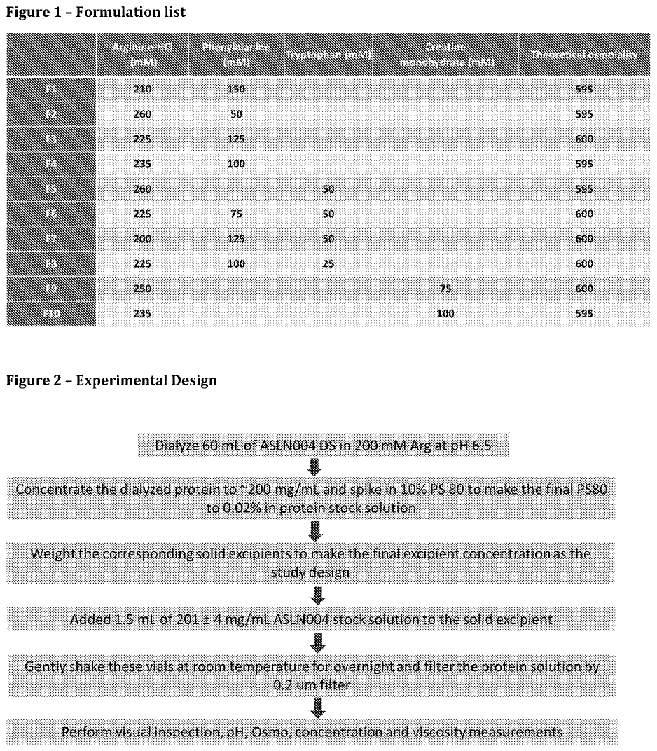

FIG. 1 shows the composition of the 10 formulations tested. The formulations were prepared according to the method shown in FIG. 2.

Briefly, 60 ml of ASLAN004 (eblasakimab) drug standard (DS) was dialyzed in 200 mM arginine-HCl and the pH adjusted to 6.5. Next, the dialyzed protein was concentrated to about 200 mg/ml and 10% polysorbate 80 solution was added to a final concentration of 0.02% w/w. Appropriate amounts of solid excipients (such as creatine monohydate) were prepared and 1.5 ml of the ~200 mg/ml eblasakimab stock solution was added to the solid excipients in a glass vial. The vials were gently shaken at room temperature overnight before the solution was filtered with a 0.2 μm filter.

The formulations were then subjected to various tests and measurements, such as a visual inspection, pH, osmolality and viscosity measurements.

The results of the tests are shown in FIG. 3 to FIG. 6.

FIG. 3 shows photographs of the formulations. As can be seen, all of the formulations tested were clear and free of particles, i.e., there was no evidence of significant aggregation.

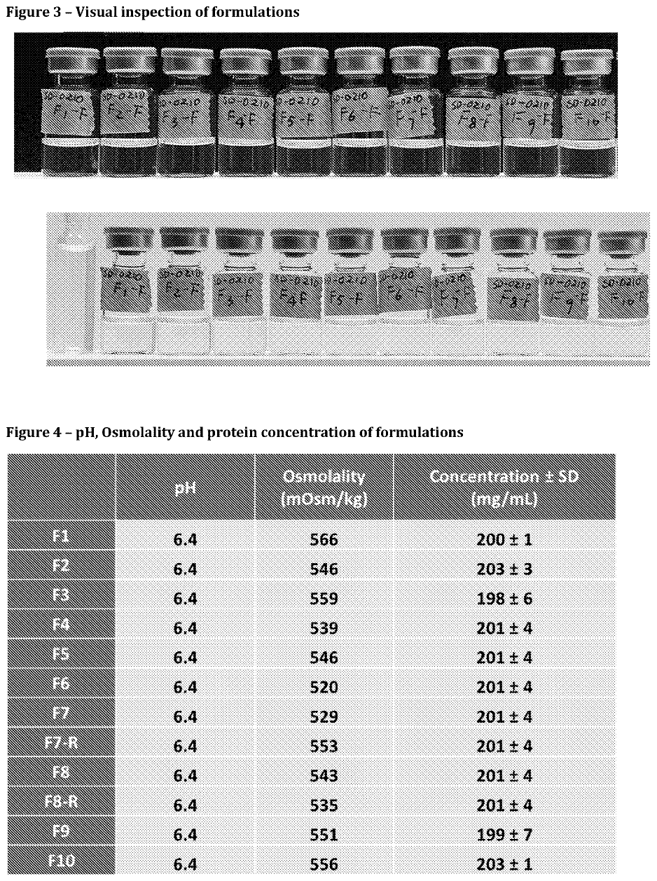

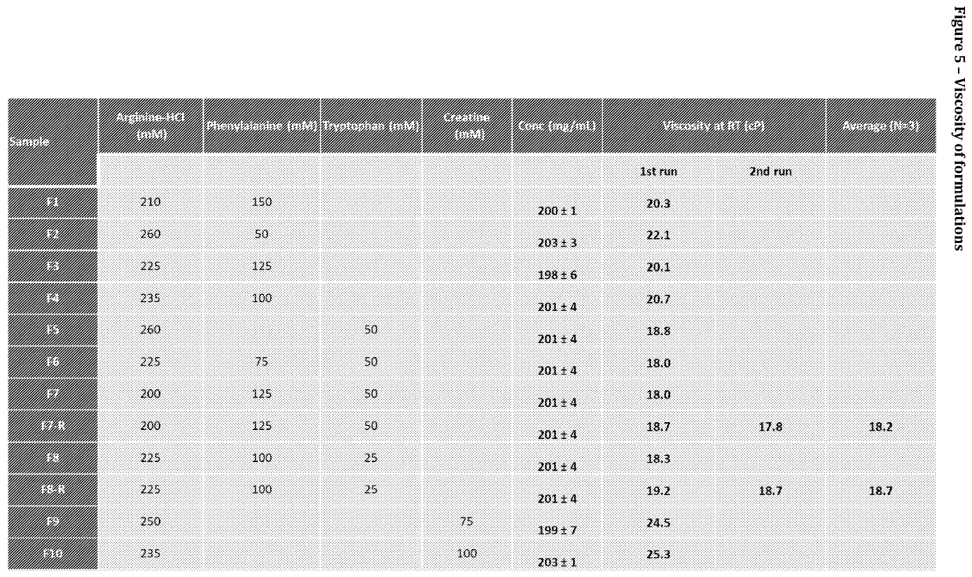

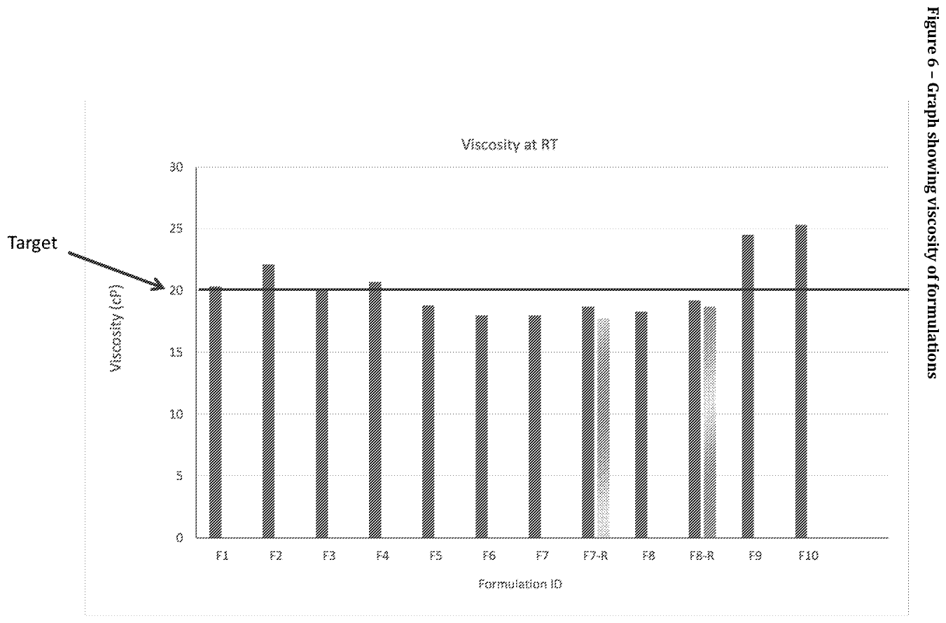

FIG. 4 shows the measured pH, osmolality and concentration of eblasakimab in the formulations. All the formulations had a pH of 6.4, an osmolaltity of about 550 mOsm/kg (measurements ranged from 520 to 566 mOsm/kg) and a concentration of about 200 mg/ml eblasakimab (measurements ranged from 192 to 205 mg/ml eblasakimab). This result suggests that the formulations had comparable pH, osmolality and antibody concentration, i.e. the formulations were suitable for a comparative experiment

FIGS. 5 and 6 show the results of the viscosity measurements. All the formulations successfully achieved the viscosity of 25 cP. In particular, the formulations comprising tryptophan, such as Formulations F5, F6, F7 and F8 achieved viscosities below 20 cP.

The results above therefore clearly demonstrate the suitability of the presently claimed high concentrations anti-IL13R antibody formulations, such as 200 mg/ml of eblasakimab.

### Example 2—Round 2 Formulation Screening

In the first round of viscosity screening, 6 formulations were found to be either at or below the viscosity target of 20 cP. A second round of viscosity screening was therefore conducted to confirm the results from round 1 and to test 3 new formulation candidates.

Table 1 below shows the 6 formulations selected from the Round 1 screen and their new names allocated for the Round 2 screen.

TABLE 1
Formulations from round 1 screen with
their new names for the round 2 screen

| Formulation selected from | New name for |
|---|---|
| round 1 screen | round 2 screen |
| F1 | F1 |
| F3 | F2 |
| F5 | F3 |
| F6 | F4 |
| F7 | F5 |
| F8 | F6 |

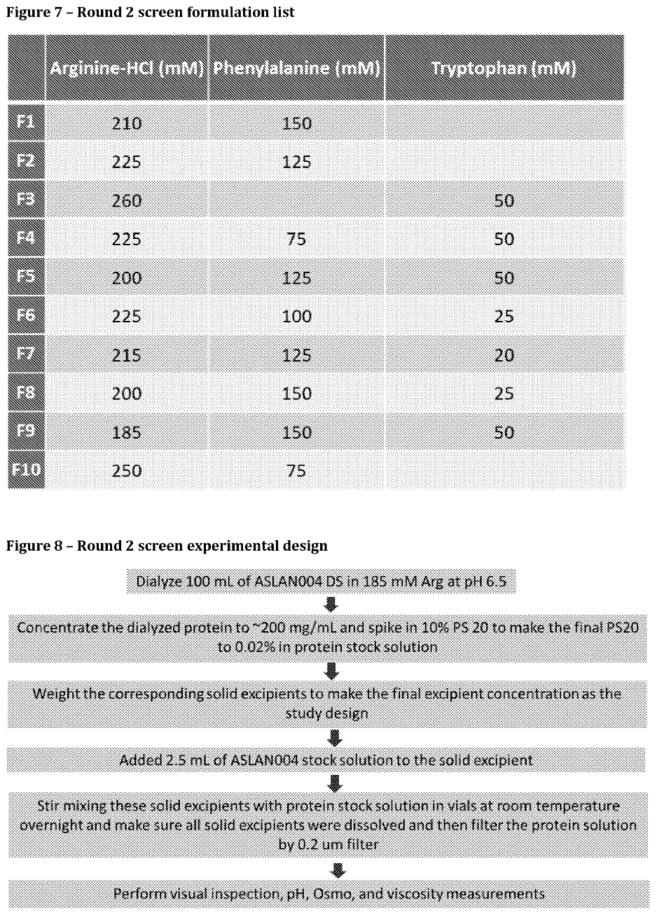

FIG. 7 shows the compositions of the formulations tested in the round 2 screen. Formulations F7, F8 and F9 are the new formulation candidates. F10 is previously developed eblasakimab formulation F46 at 200 mg/ml. The composition of F46 is shown in Table 2 below. This formulation was included as a control for comparison with the presently claimed formulations.

All formulations were successfully prepared using a similar protocol as in round 1. Details of the preparation method are shown in FIG. 8. The formulations were subjected to various tests and measurements, such as a visual inspection, pH, osmolality and viscosity measurements as per the round 1 screen.

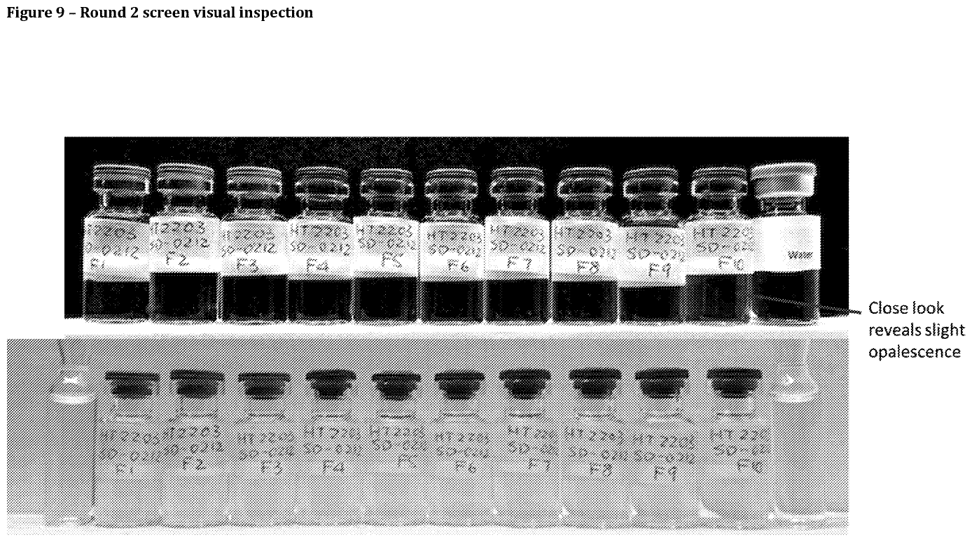

The results of the round 2 screen are shown in FIGS. 9 to 12.

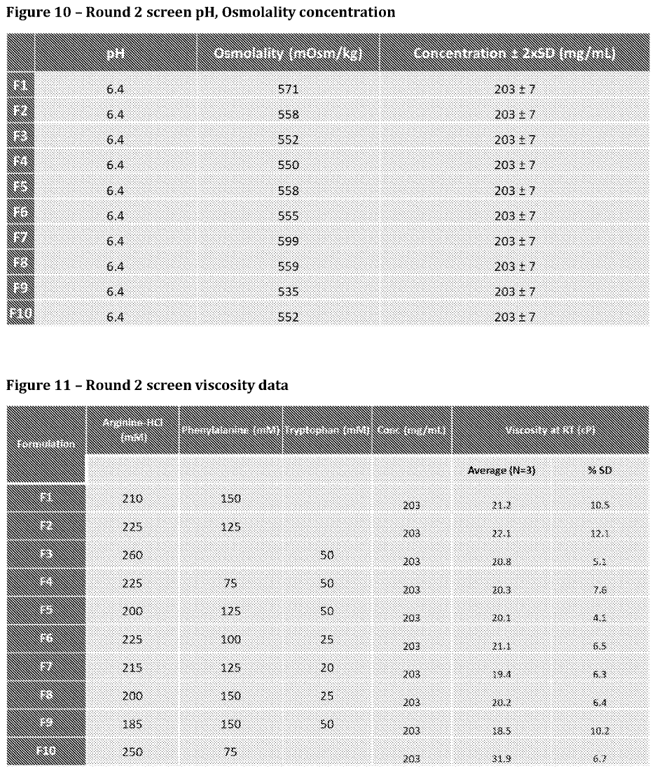

FIGS. 9 and 10 show the results of the visual inspection, pH and osmolality for the round 2 screen. The pH, Osmolality, and visual appearance of all formulations were found to be acceptable for viscosity testing. In particular, all of the formulations were free of visible particles. Control formulation F10 (F46) however appeared to be slightly opalescent in comparison with the newly developed formulations.

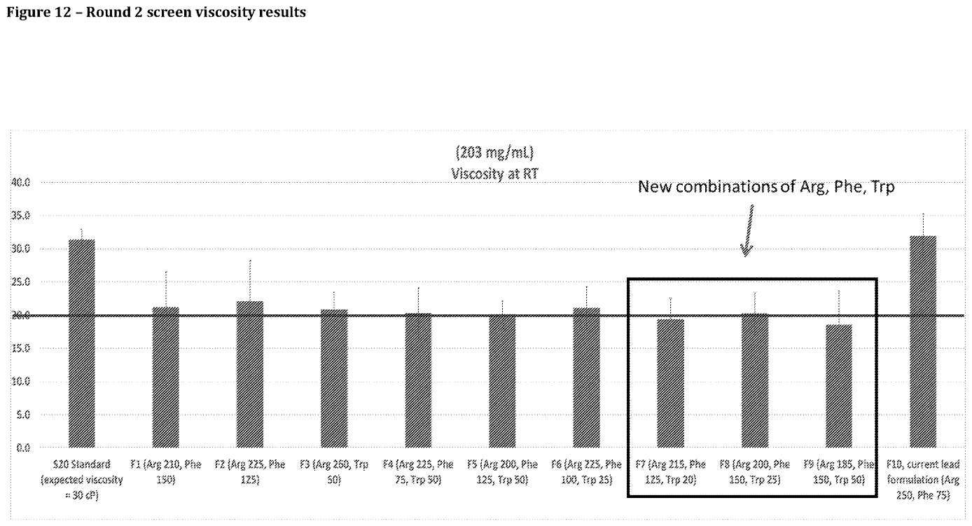

FIGS. 11 and 12 show the results of the viscosity measurements for the round 2 screen. The measured viscosity of the reference standard (see FIG. 12) was found to be 1.4 cP above the expected value (31.4 cP vs 30 cP) but within the acceptable margin of error. Viscosity of the F10 formulation was previously measured at 29.5 cP (n=1) at 200 mg/mL. This formulation was found to be 31.9 cP (n=3) at 203 mg/mL in the present screen, suggesting that the method used to of measure the viscosity was reliable.

Overall, all the formulations with the exception of F10 had good viscosity close to or under the target value of 20 cP. In particular, the formulations with the Arg, Phe, and Trp combination tended to perform the best. All the round 2 formulations are therefore expected to be injectable at room temperature.

Protein concentration in Round 2 is slightly higher than in Round 1 (average of 203 mg/mL versus 200 mg/mL in Round 1), which may explain the slight increase in viscosity (~1-2 cP) of the same formulations tested in Round 1.

The new formulation candidates (F7, F8, F9) also show promise as a slight further reduction in viscosity was achieved with F7 and F9 compared to the other round 2 formulations.

### Example 3—Stability Study Results at 4 Weeks

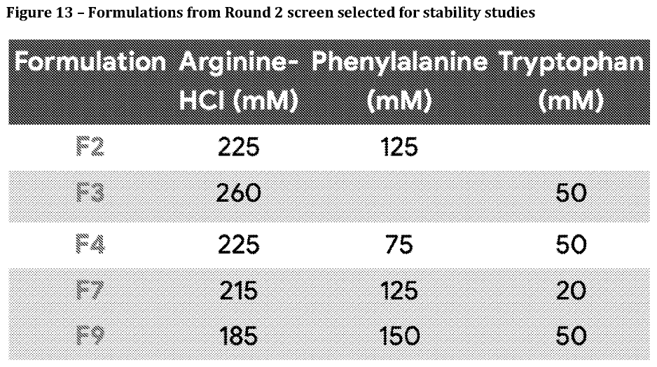

Based on the round 2 screening results, 5 formulations were selected for preliminary stability studies. Their compositions are shown in FIG. 13. Formulation 3 was tested at both 195 mg/ml (F3-195) and 200 mg/ml (F3-200) target eblasakimab concentrations.

The formulations were stored in vials at either 4° C., 25° C. (room temperature) or 40° C. (accelerated thermal stress conditions) and subjected to various tests after 4 weeks of storage (T=4 weeks).

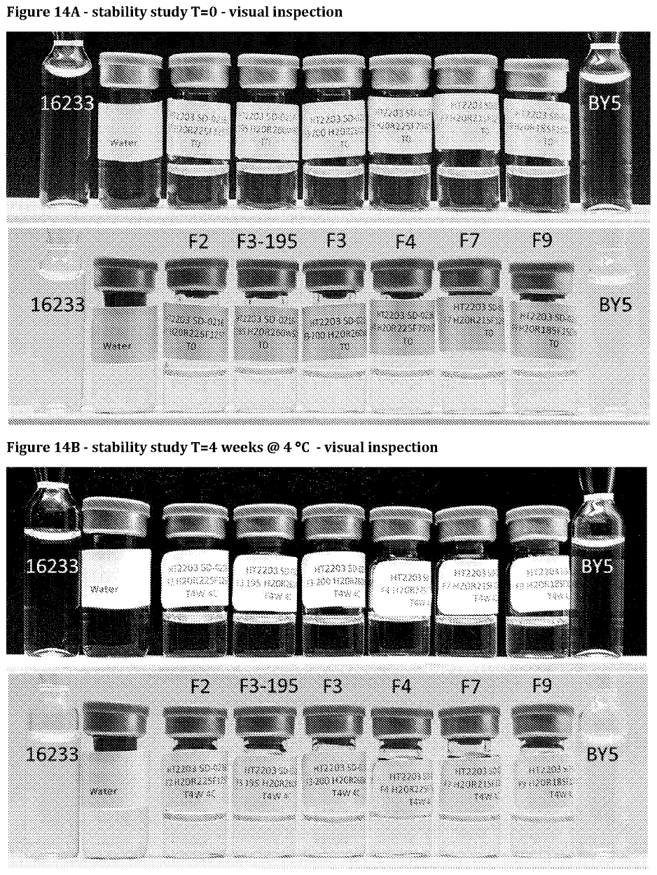

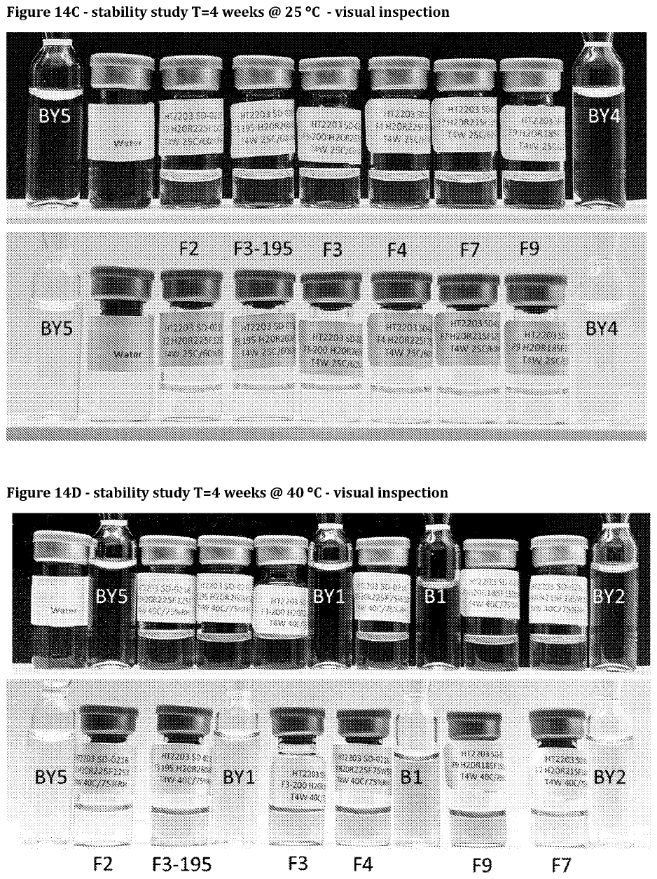

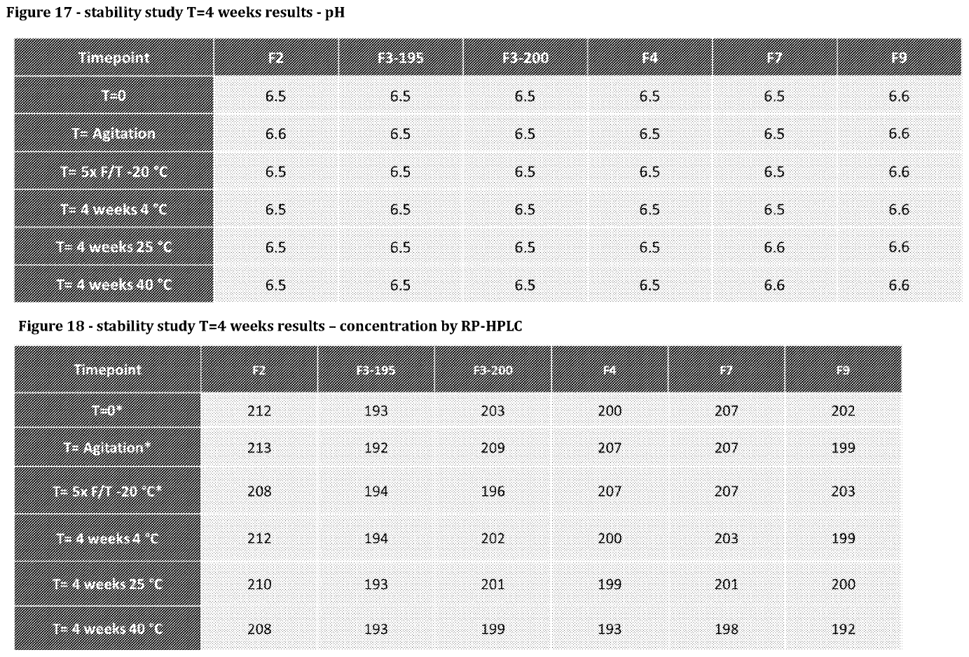

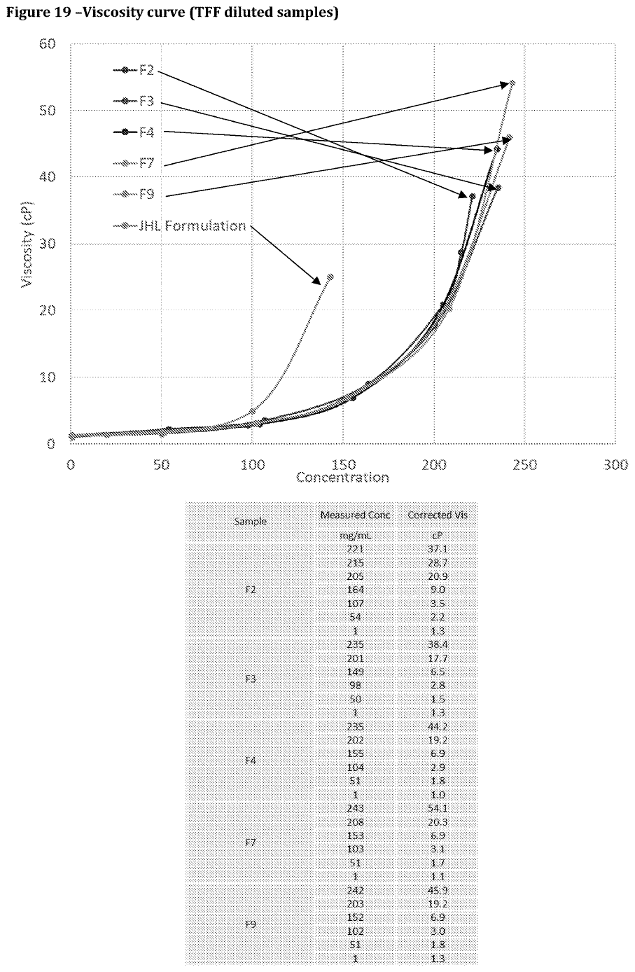

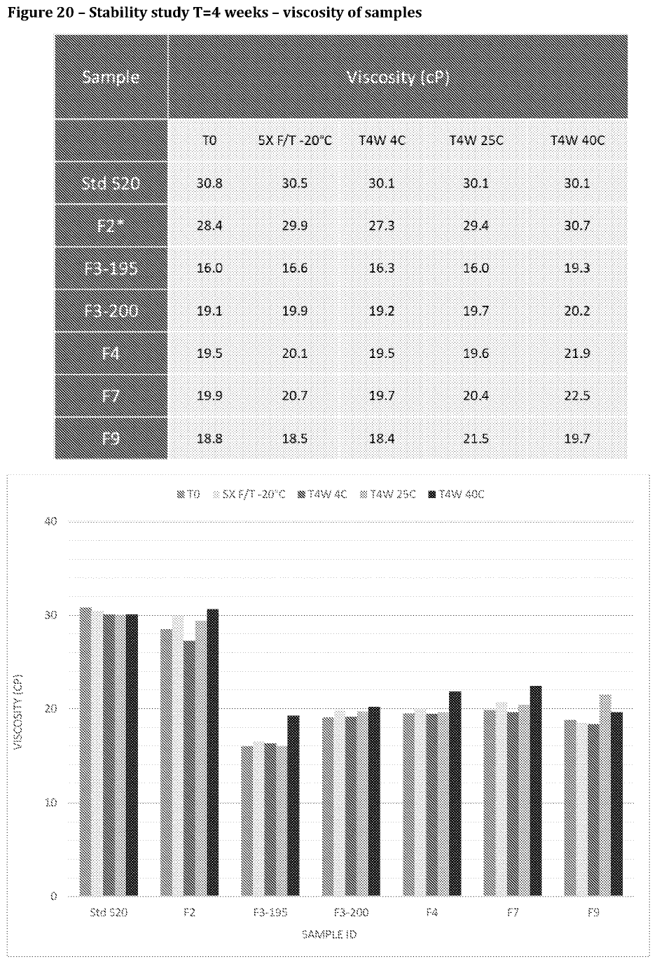

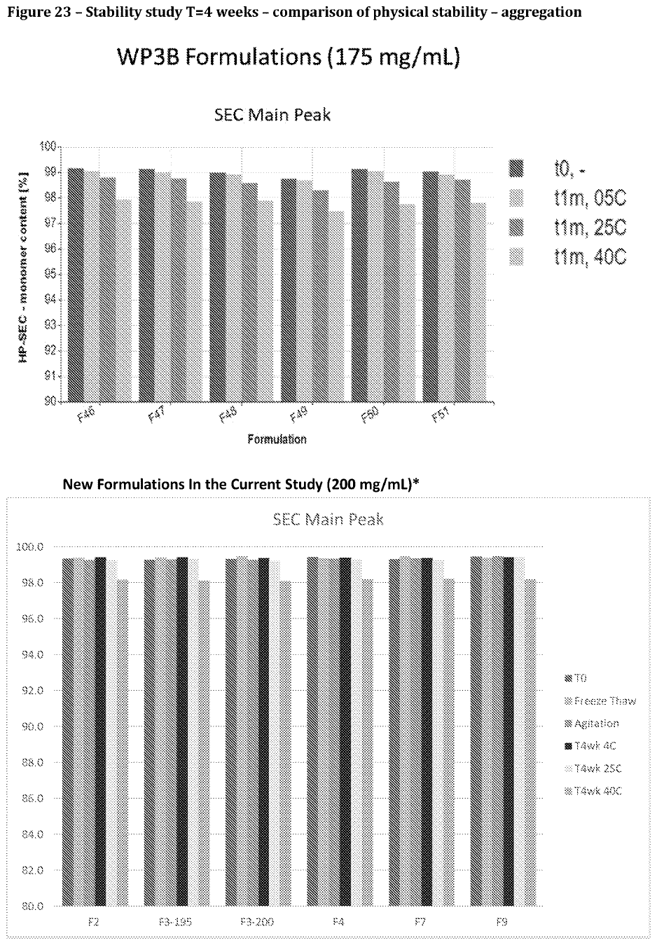

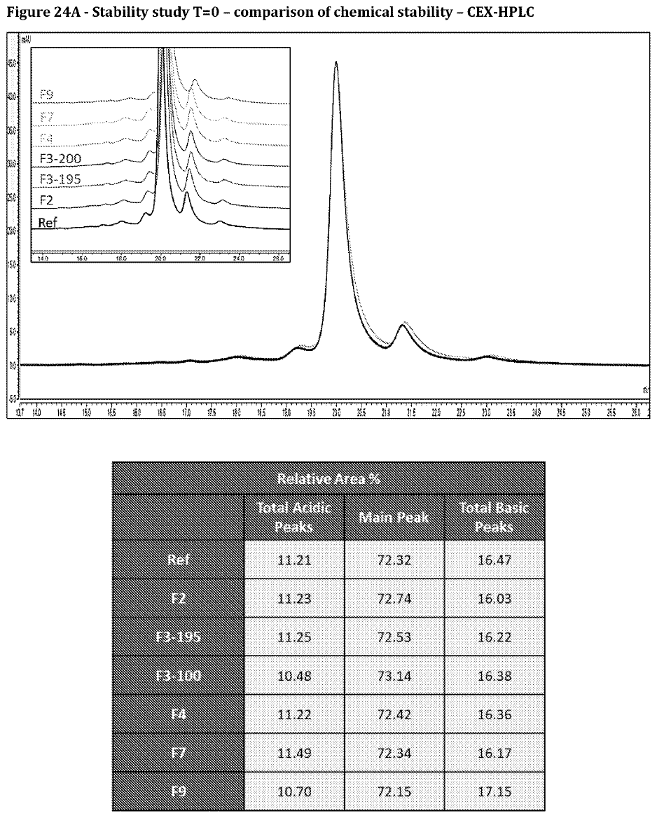

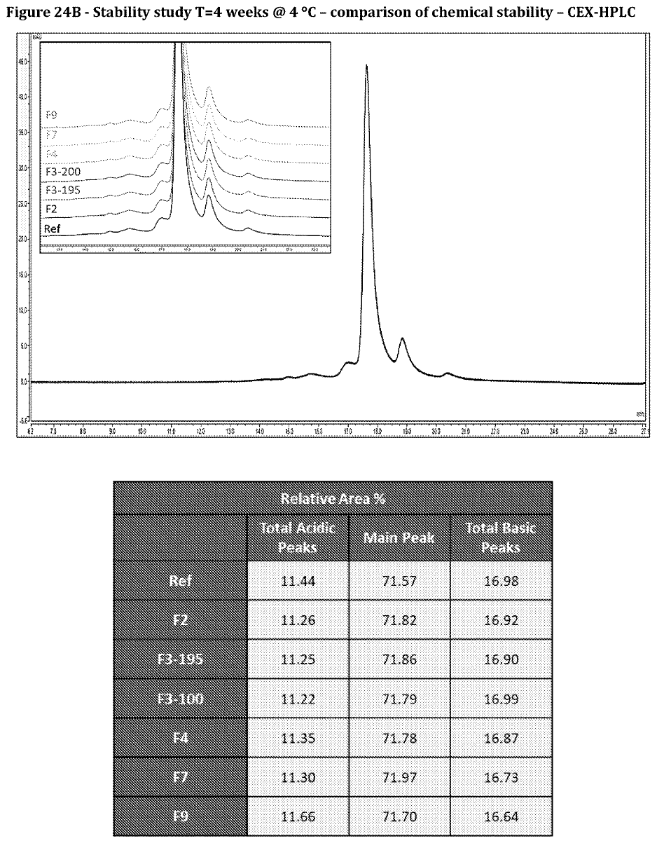

The results of the tests are shown in FIGS. 14 to 27.

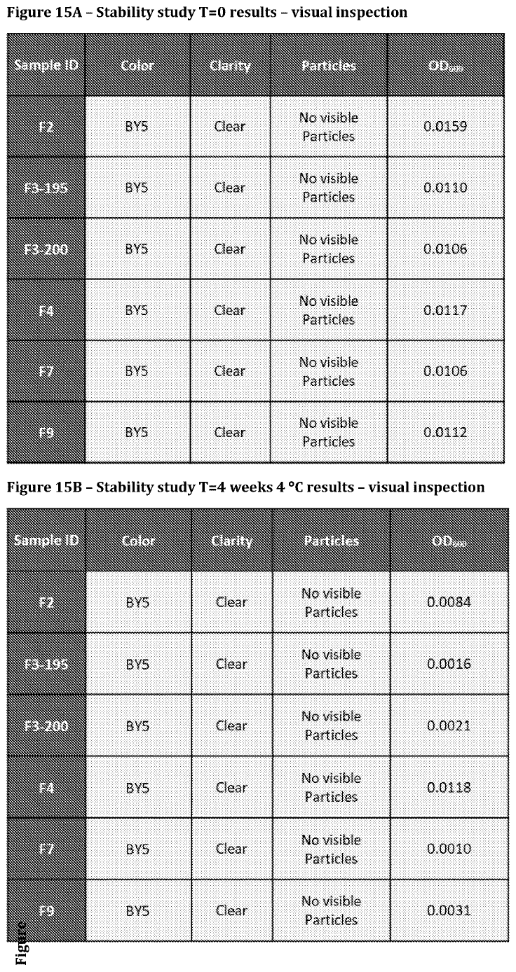

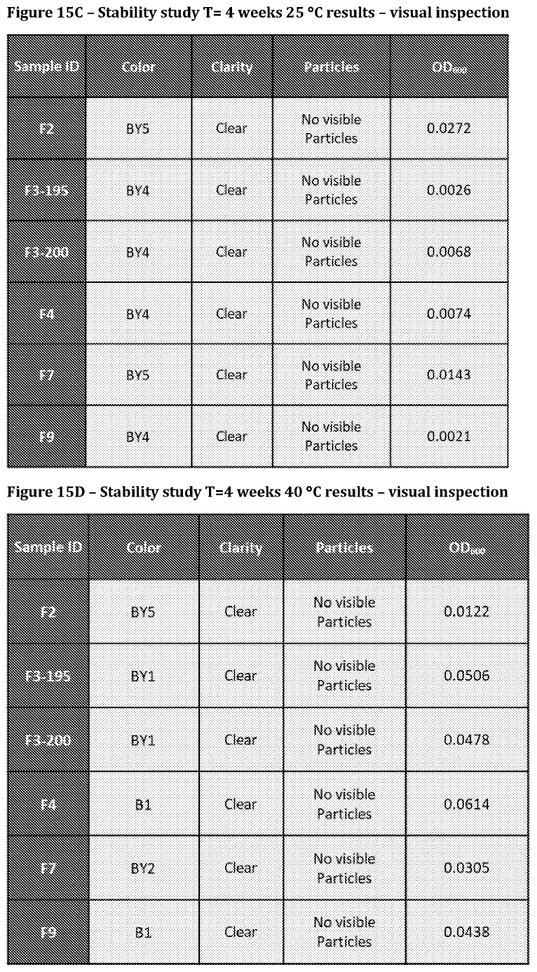

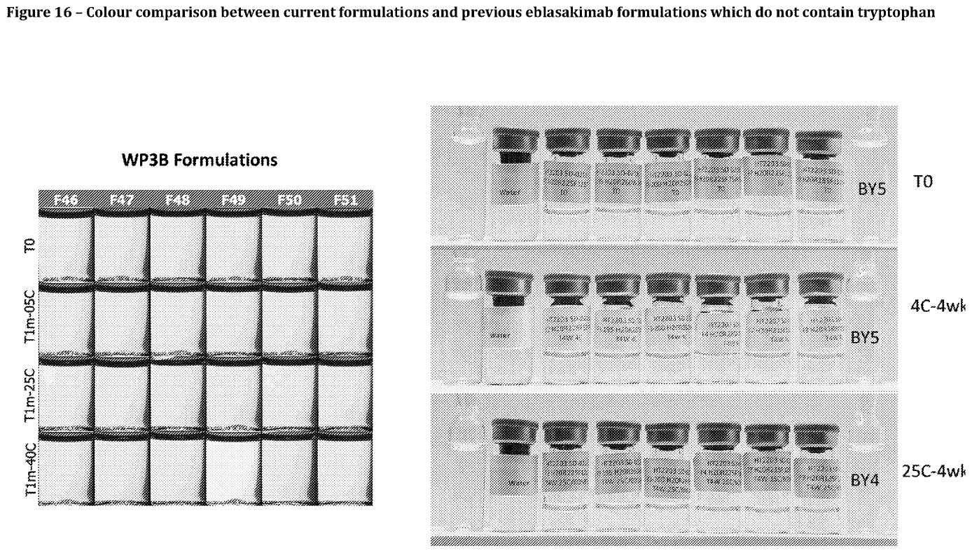

FIGS. 14 to 16 show the results of the visual inspection. No signs of visible particle formation in any of the formulations even after exposure to accelerated thermal stress conditions. The formulations stored at 40° C. (See FIG. 14D and FIG. 15D) showed signs of yellowing Formulation F2, which lacks tryptophan and F7, which had 20 mM Trp instead of 50 mM Trp showed slightly less yellowing. FIG. 16 shows a comparison between the 5 current formulations vs previous eblasakimab formulations (WP3B). Details of the compositions of the previous formulations are shown in Table 2 below.

TABLE 2
Composition of previous eblaskimab formulations (WP3B)

| | API | | | Arg- | | | | Theoretical |
|---|---|---|---|---|---|---|---|---|
| | [mg/ | | His | HCI | Phe | Additional | | osmolality |
| No | ml] | pH | [mM] | [mM] | [mM] | Excipients | PS20 | [mOsmol/kg] |
| F46 | 175 | 6.5 | 20 | 250 | 75 | | 0.02 | 600 |
| F47 | 175 | 6.5 | 20 | 175 | 80 | | (w/v) | 455 |
| F48 | 150 | 6.5 | 20 | 175 | | 50 mM | % | 525 |
| | | | | | | CaCl₂ | | |
| F49 | 175 | 6.5 | 20 | 100 | | 180 mM | | 405 |
| | | | | | | Sucrose | | |
| F50 | 175 | 6.5 | 20 | 175 | 75 | 50 mM | | 600 |
| | | | | | | CaCl₂ | | |
| F51 | 175 | 6.5 | 20 | 200 | 50 | | | 475 |

There was no sign of yellowing in any of the formulations under normal storage conditions, including the WP3B formulations stored at 40° C. Hence, the present inventors believe that the yellowing is most likely due to Trp degradation at high temperatures.

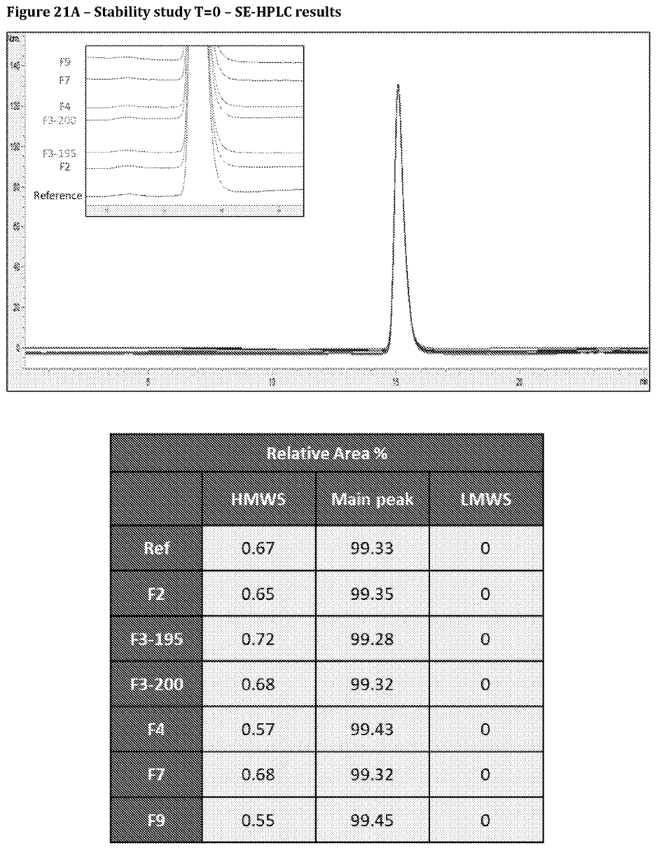

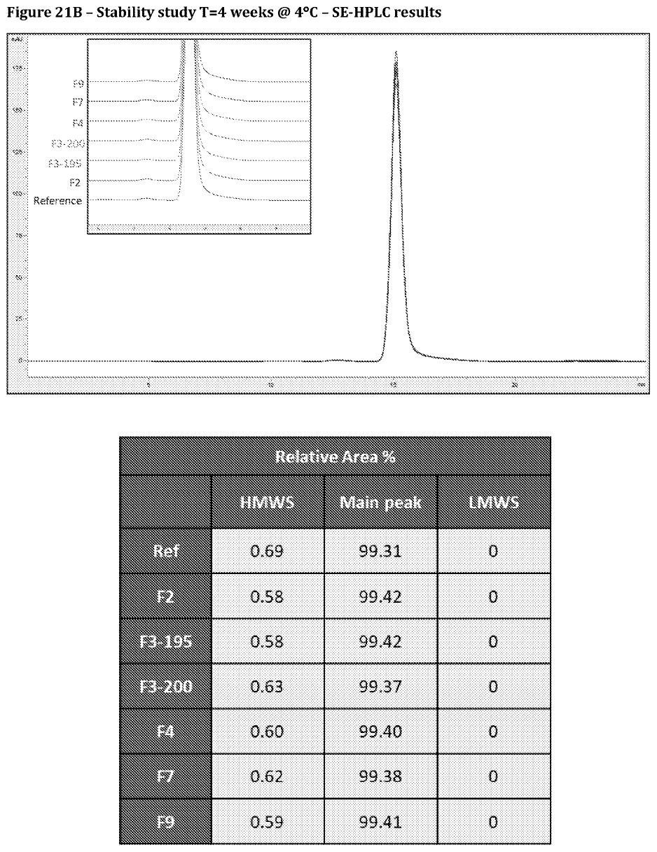

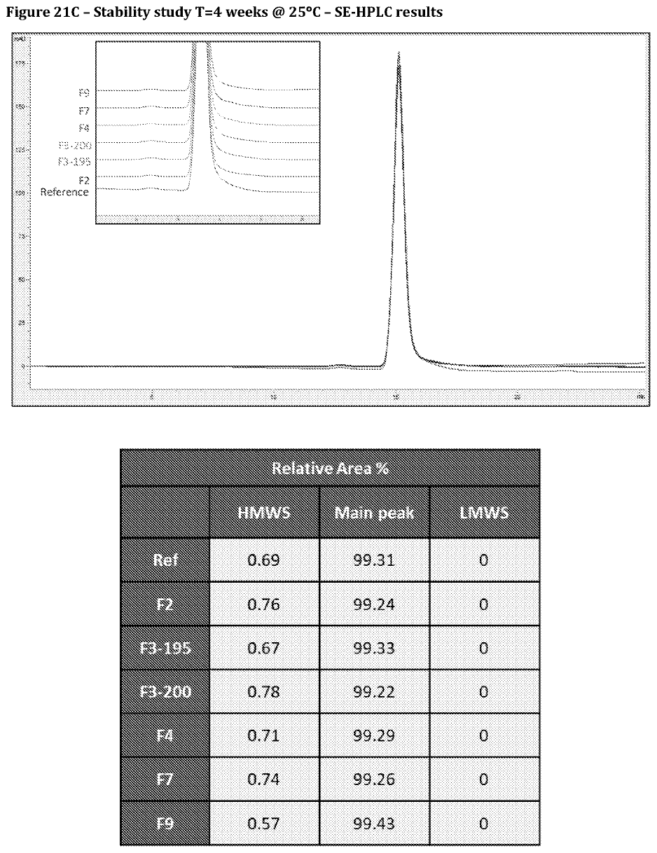

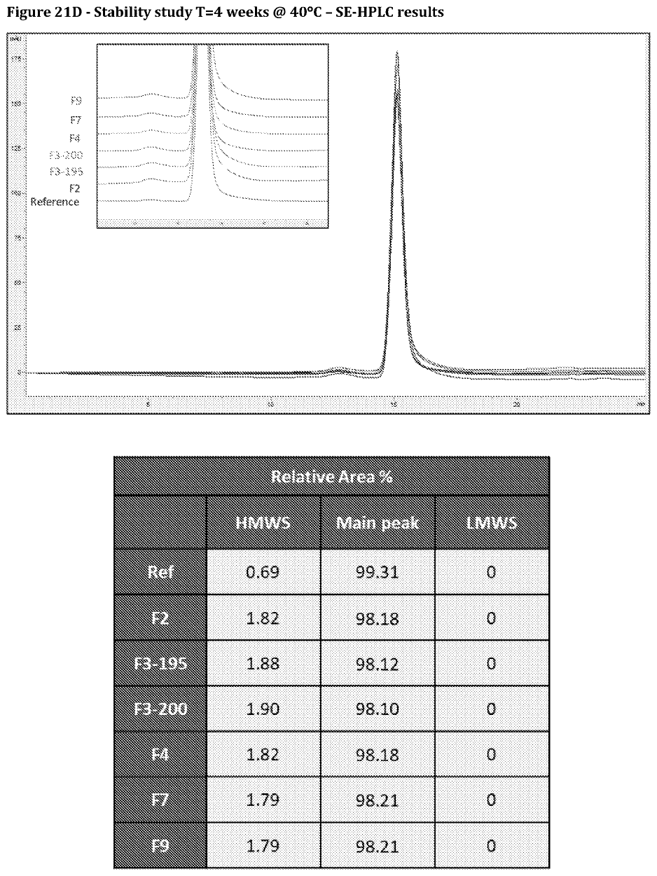

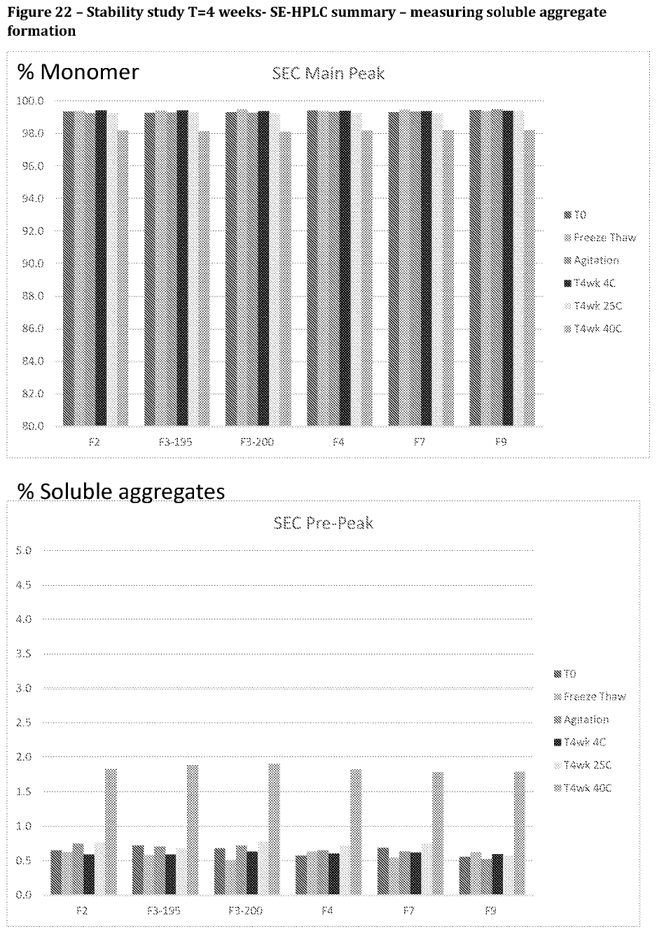

FIGS. 21 and 22 show the SE-HPLC results which are indicative of the rate of aggregation, which is in turn an indication of the physical stability of the formulations. The SE-HPLC results suggest that there was no significant difference among the formulations in the rate of aggregation. All the 5 formulations perform favorably in comparison with the previous eblasakimab formulations (WP3B).

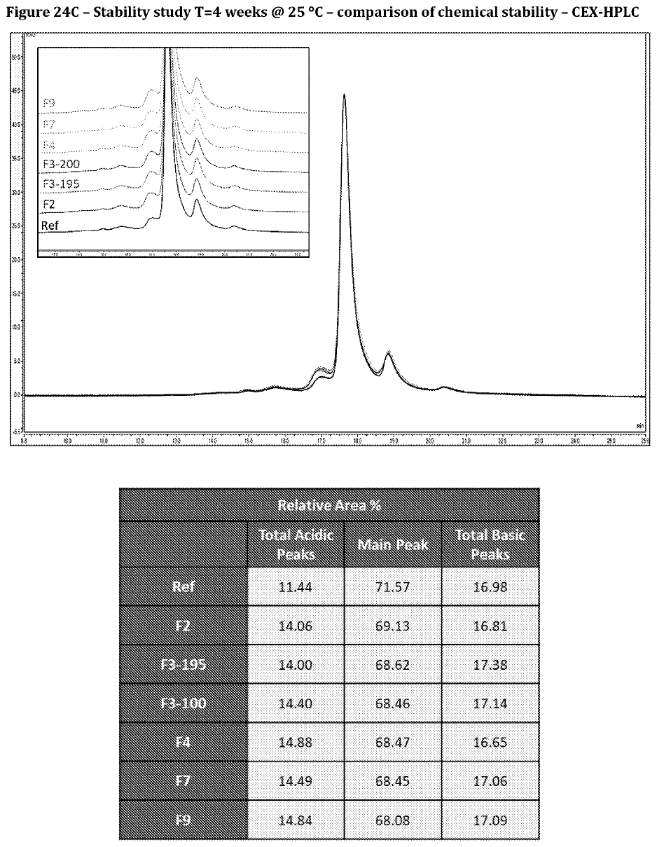

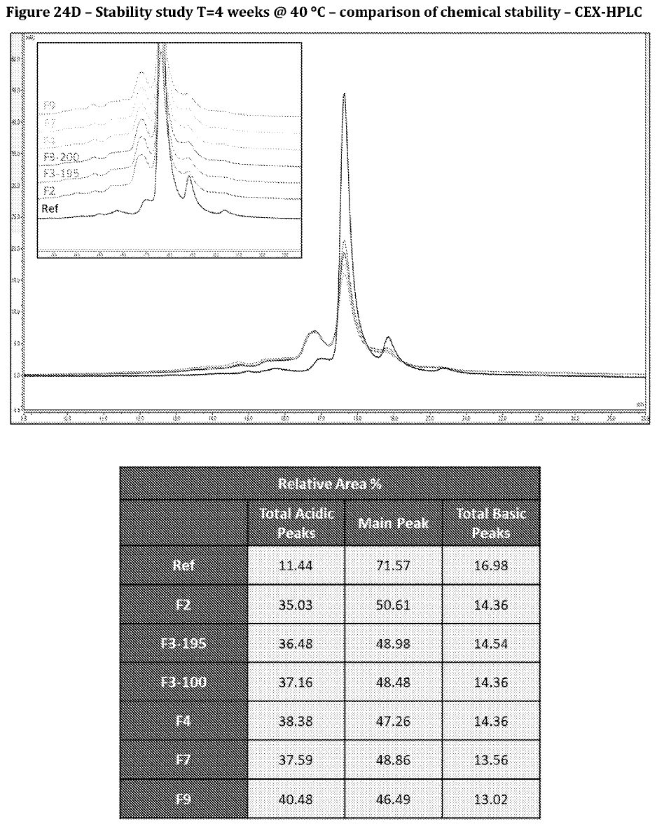

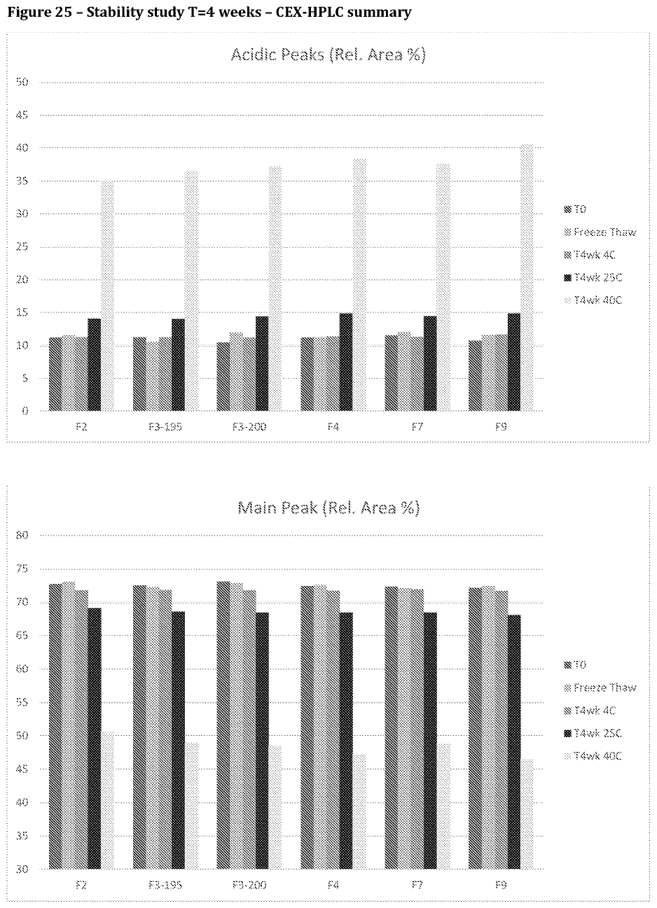

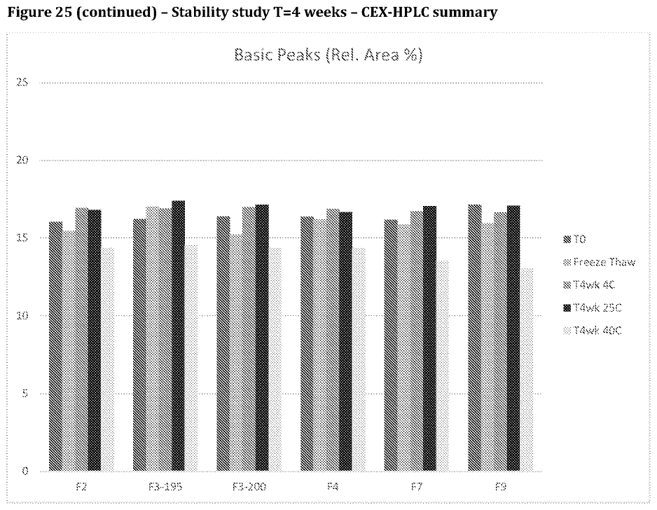

FIGS. 24 and 25 show the CEX-HPLC results which indicate the degree of deamidation, which in turn is indicative of the chemical stability of the formulations. The results suggest there was no significant difference in the rate of chemical degradation (deamidation) between formulations. Unfortunately, it was not possible to compare the 5 formulations with the previous eblasakimab formulations (WP3B) because no T=4-week data from these formulations was not available.

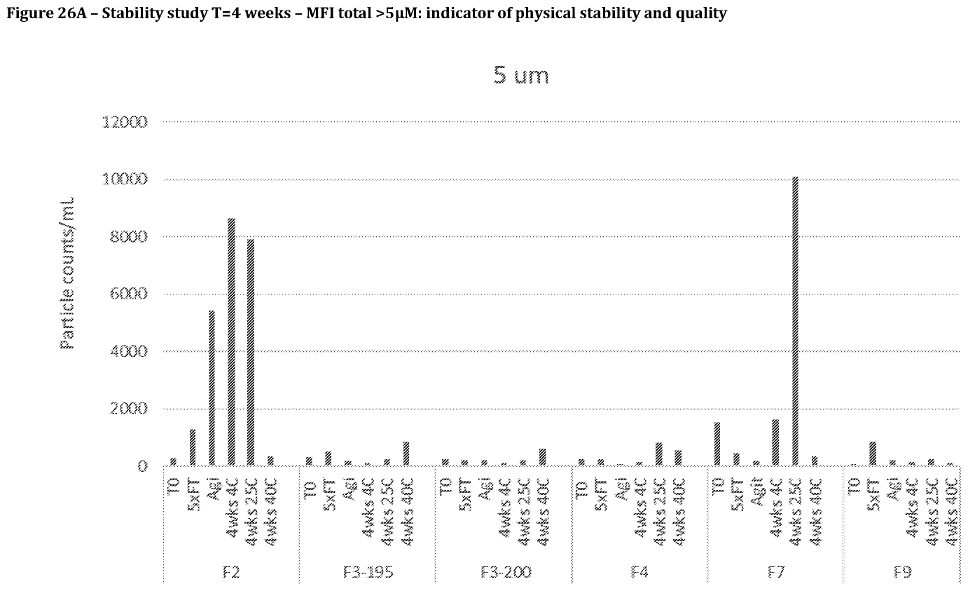

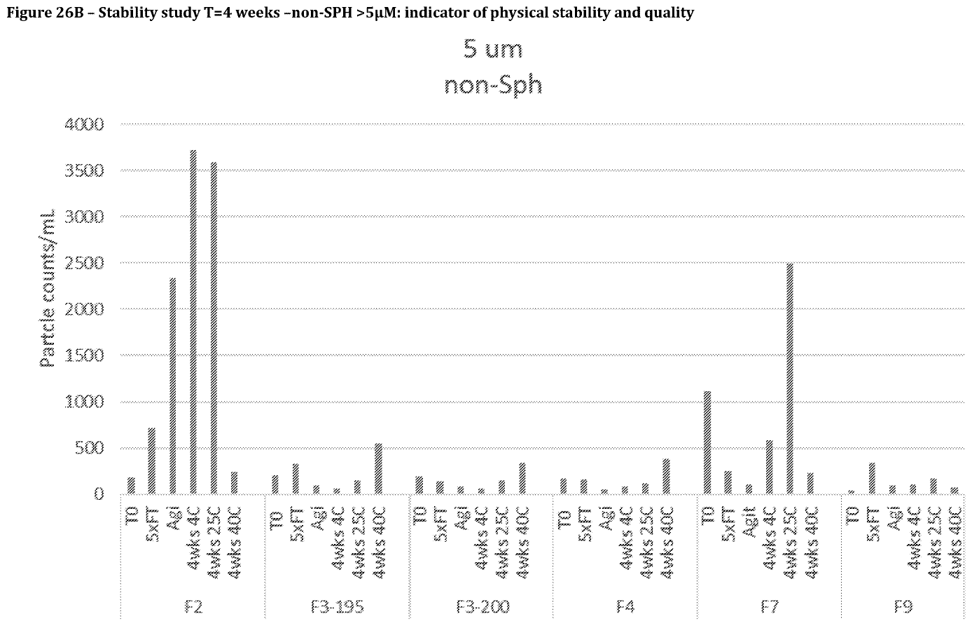

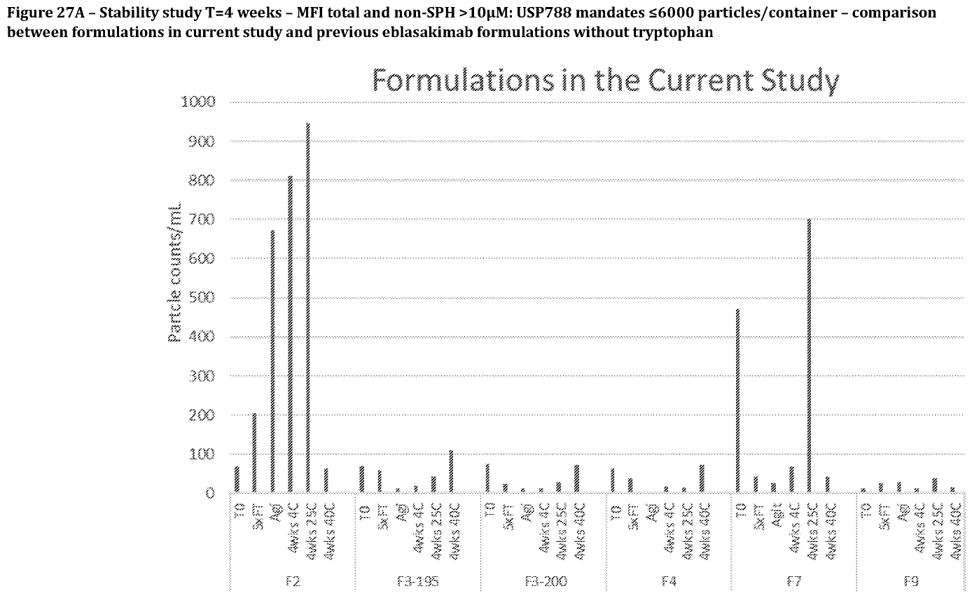

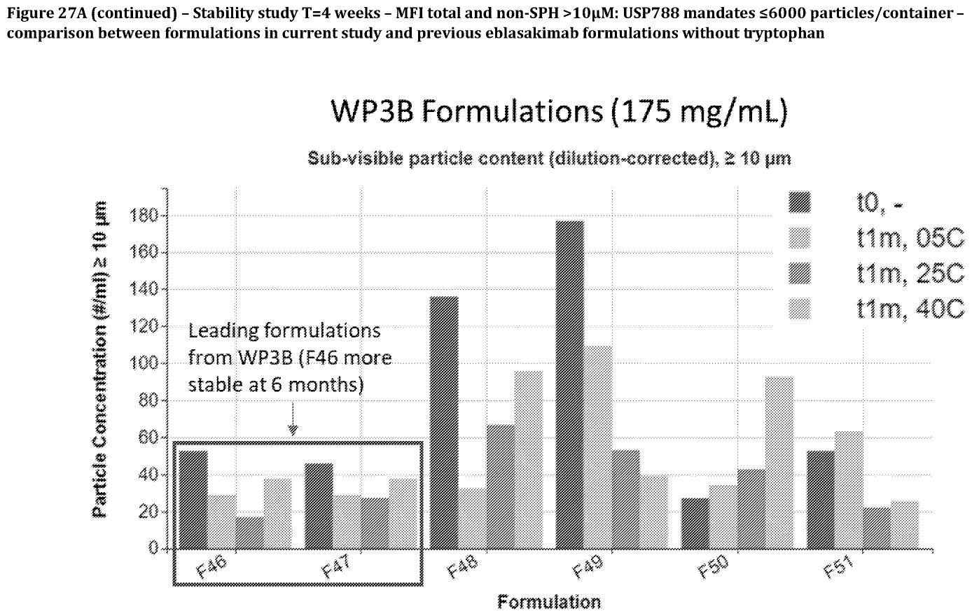

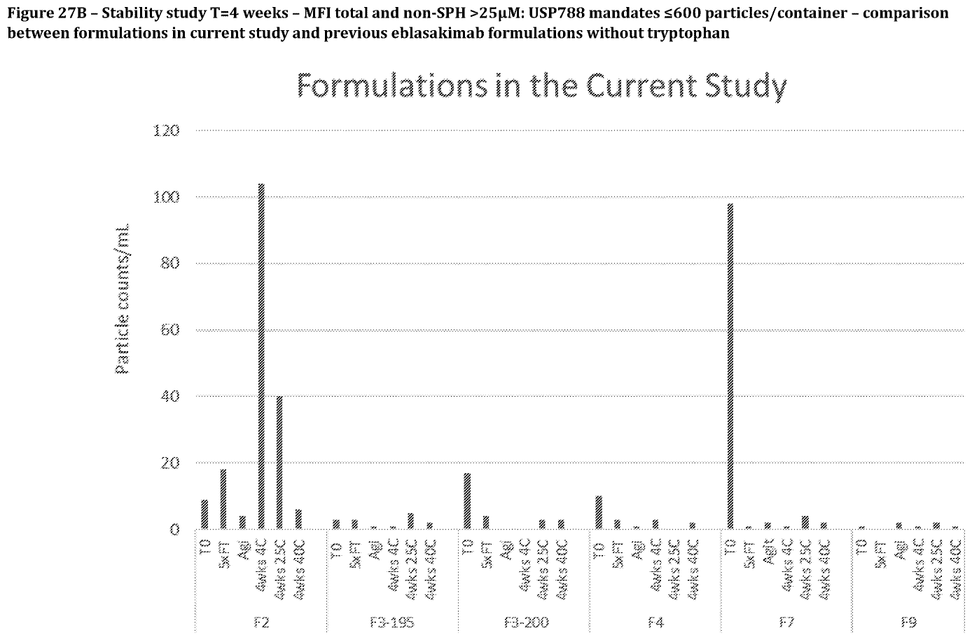

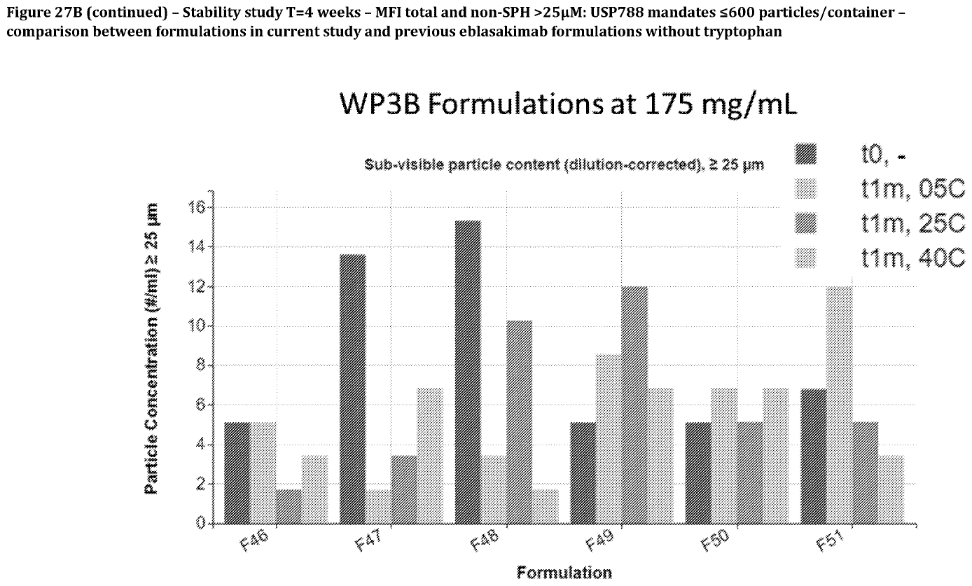

FIGS. 26 and 27 show the results of micro-flow imaging (MFI) and non-spherical (non-SPH) analysis. These tests indicate the propensity for the formulations to form subvisible particles (SVPs), which in turn is an indicator of the physical stability and quality of the formulations. The results suggest that formulations F2 and F7 show greater propensity for particle formation. The rest of the formulations are highly resistant to particle formation during storage at normal storage temperatures as well as accelerated stress conditions.

Importantly, the USP 788 standard for subvisible particles requires that for >10 μM, there should be ≤6000 particles/container and for >25 μM, there should be ≤600 particles/container. The results suggest that all the formulations tested would satisfy the USP 788 standard, including the formulations exposed to accelerated stress conditions.

Overall, the week 4 stability results suggest that formulations that contain 50 mM Trp are less viscous and more stable (less subvisible particles) than those with less or no Trp.

In summary, based on week 4 stability data, the new 200 mg/mL formulations show promising stability in comparison with formulations that were previously developed.

### Example 4—Stability Study Results at 8 Weeks

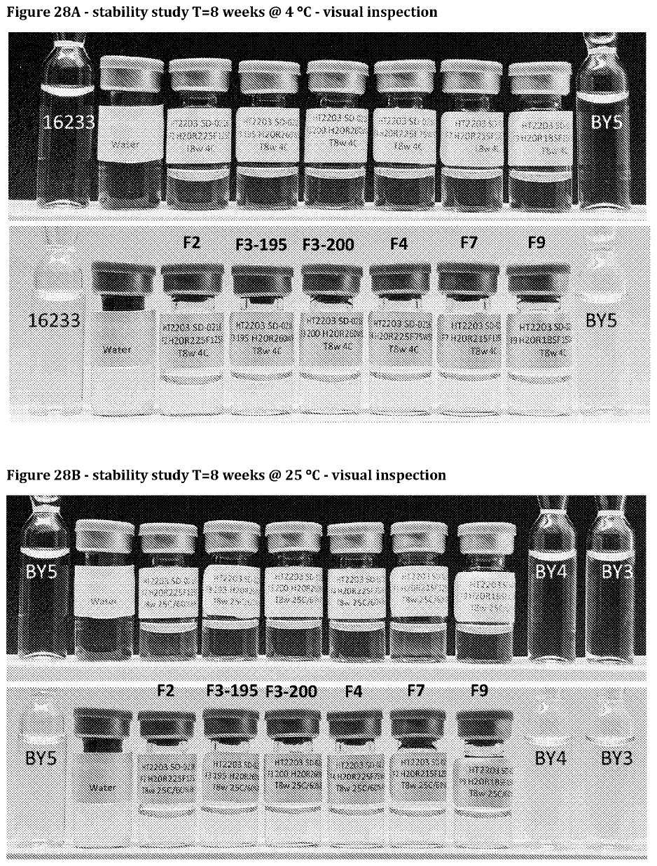

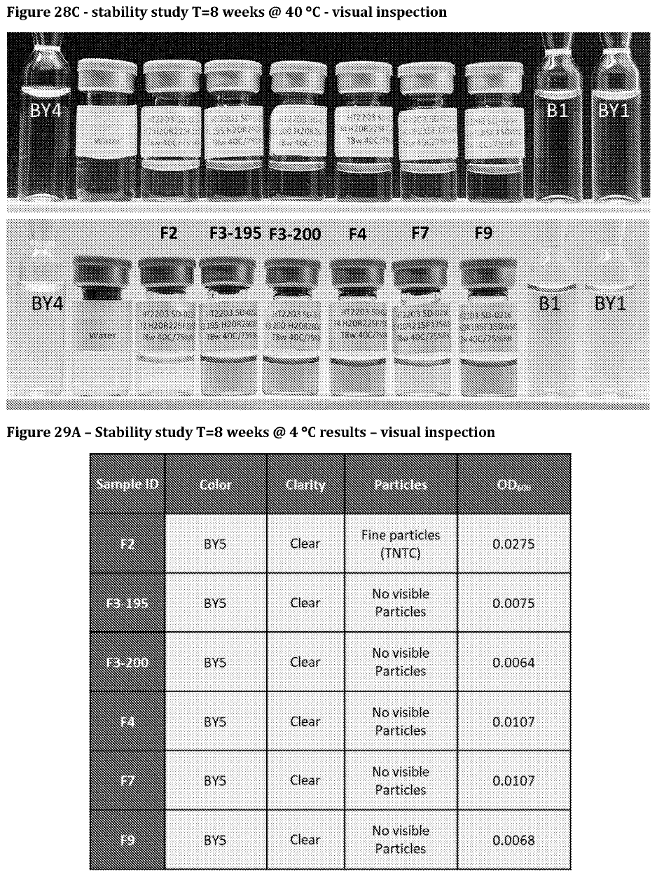

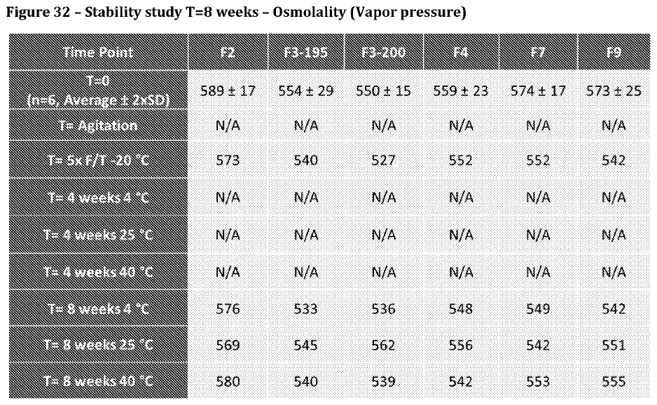

After 8 weeks of storage (T=8 weeks), the stability of the 5 formulations was further assessed by performing similar tests to those described in Example 3. The results of the tests are shown in FIGS. 28 to 38.

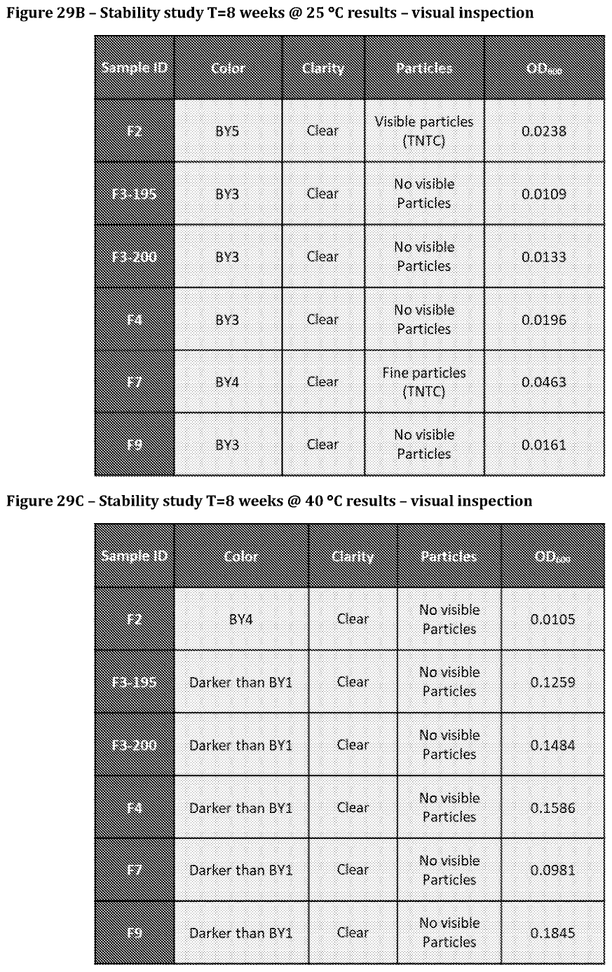

FIGS. 28 to 29 show the results of the visual inspection. As compared to the 4-week results, a large number of fine particles were now visible in formulation F2 when stored at 4° C. and in formulations F2 and F7 when stored at 25° C. The remaining formulations had no visible particles.

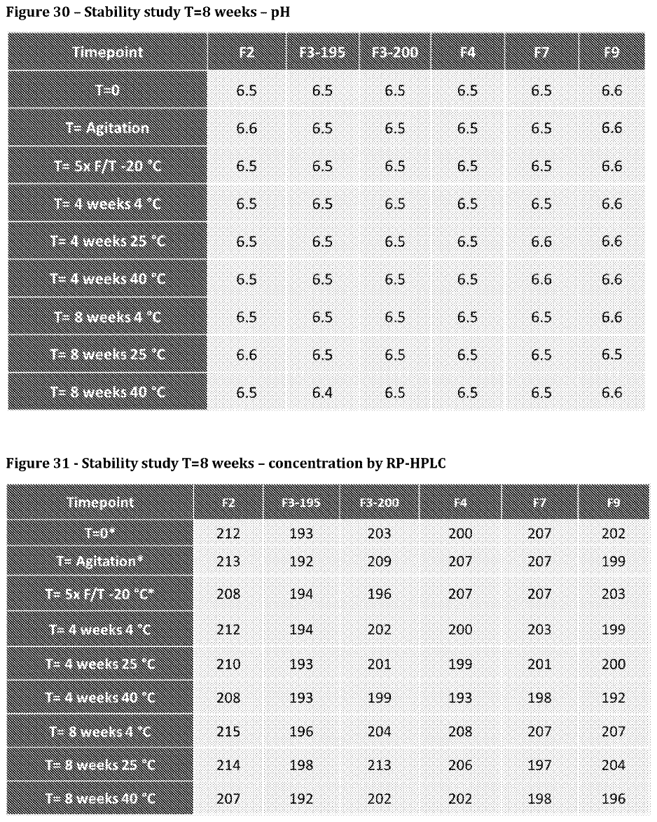

FIGS. 30 to 32 show the pH, concentration and osmolality of the formulations when measured at T=0, T=4 weeks and T=8 weeks. The results indicate no significant changes in the pH, concentration and osmolality over the course of the 8-week study, indicating that these aspects of the formulations continue to remain stable.

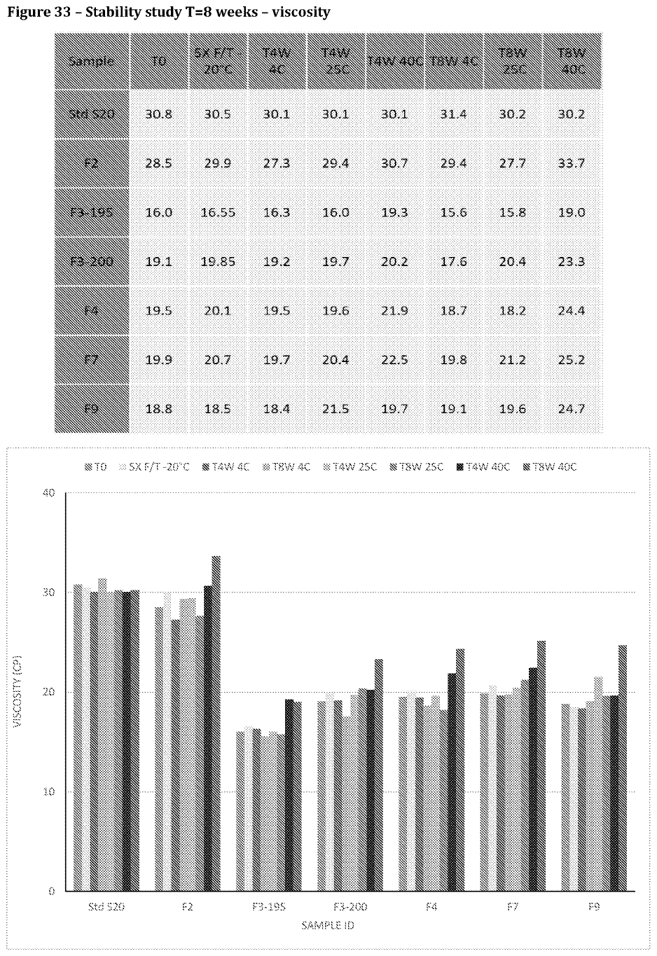

FIG. 33 shows the viscosity results for the formulations when measured at T=0, T=4 weeks and T=8 weeks. With the exception of formulation F2, all the other formulations had viscosity readings close to or below the target of 20 cP at 8 weeks when stored under at 4° C. and 25° C. Formulation F3 had the best viscosity, with the 195 mg/ml formulation (F3-195) continuing to show a viscosity below 20 cP even under accelerated stress conditions (40° C.).

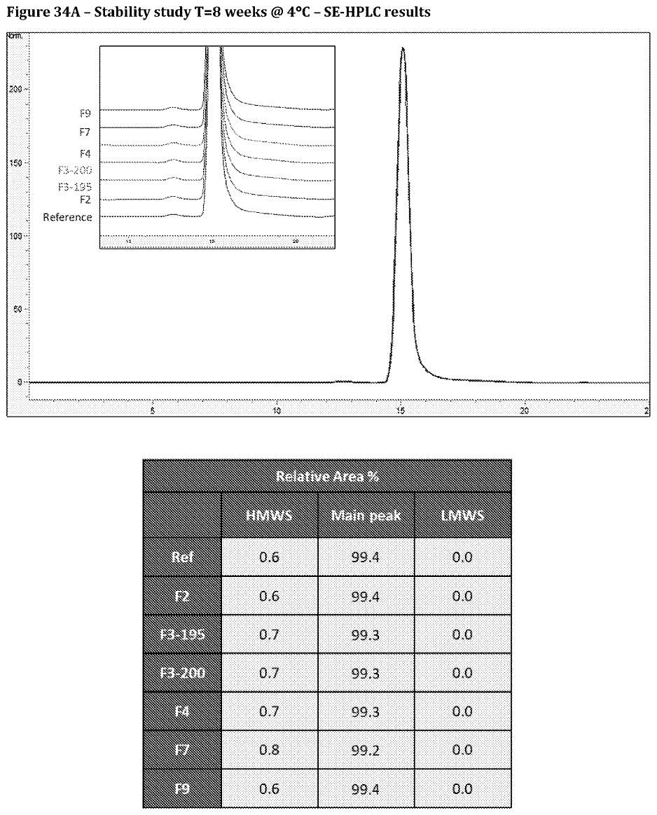

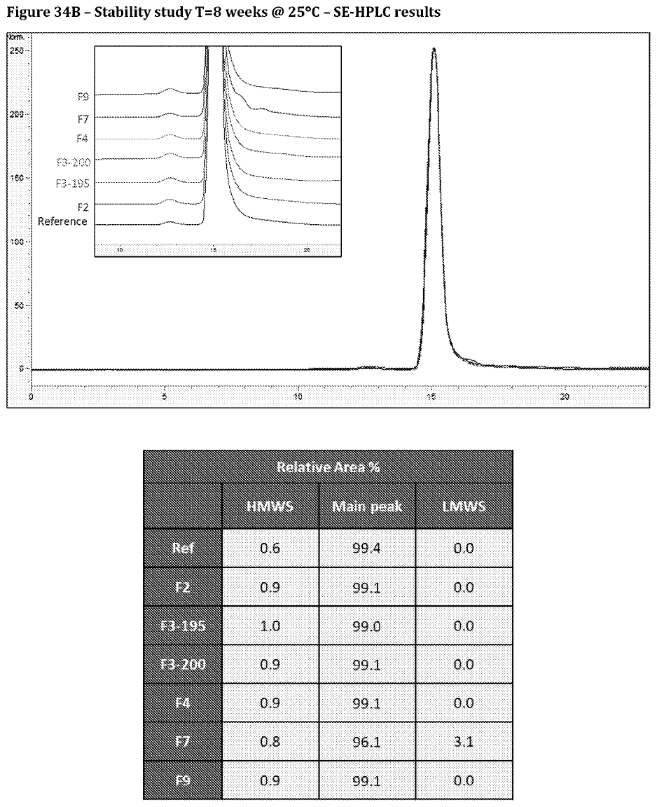

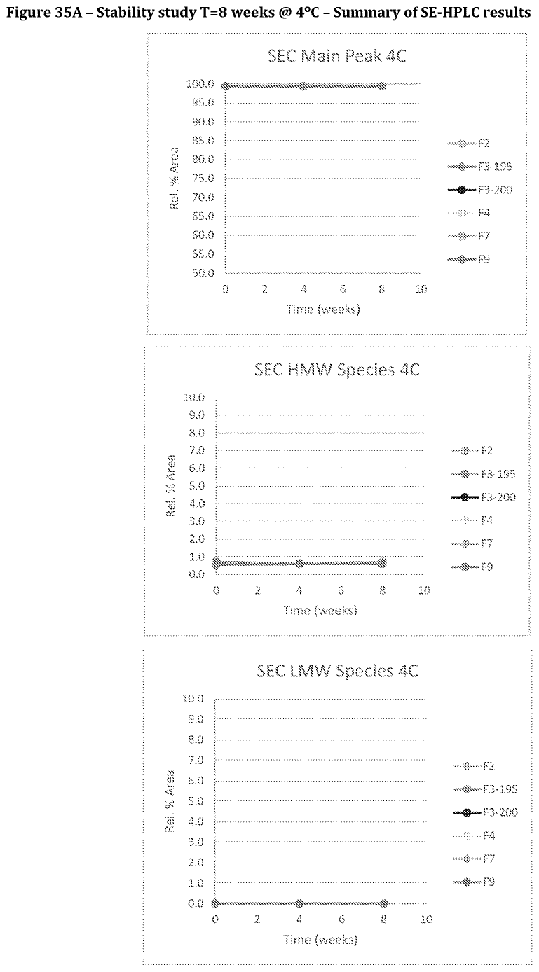

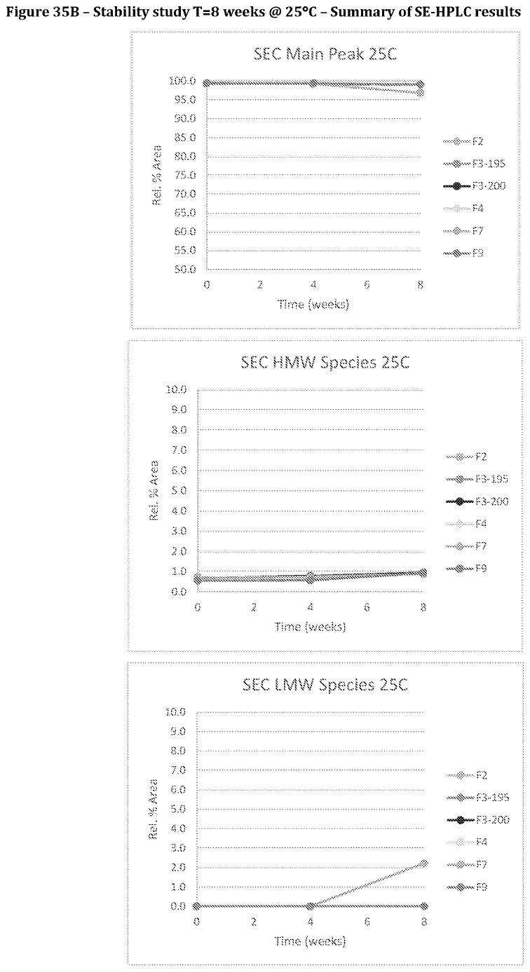

FIGS. 34 to 35 show the SE-HPLC results at T=8 weeks. The results suggest that when stored at 4° C., all the formulations had excellent aggregation stability at 8 weeks. When stored at 25° C., all the formulations, with the exception of formulation F7, showed almost no loss of purity at 8 weeks.

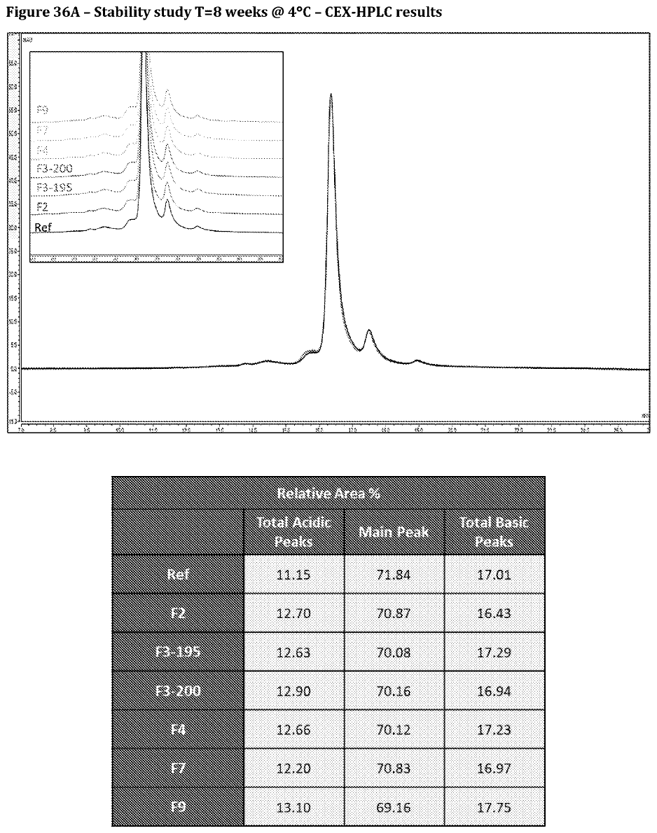

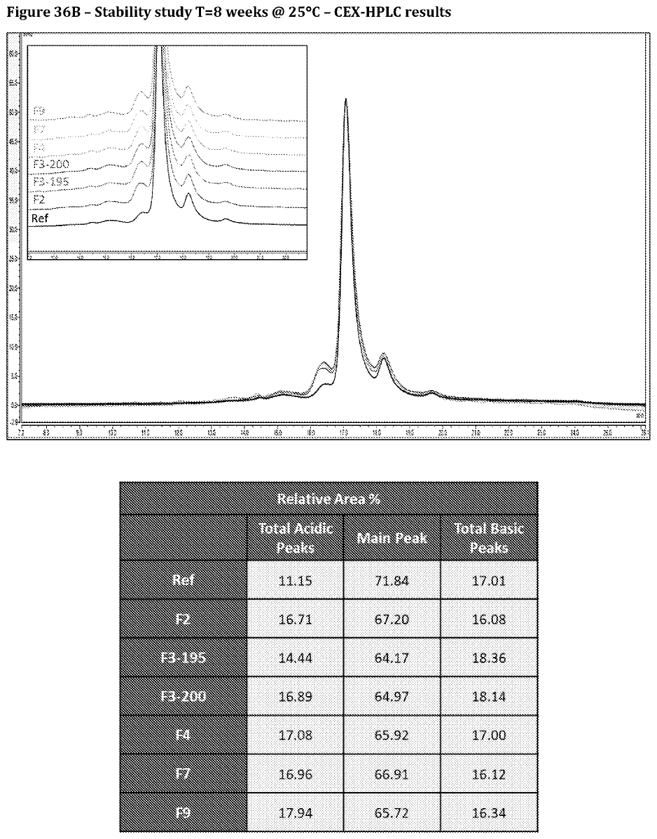

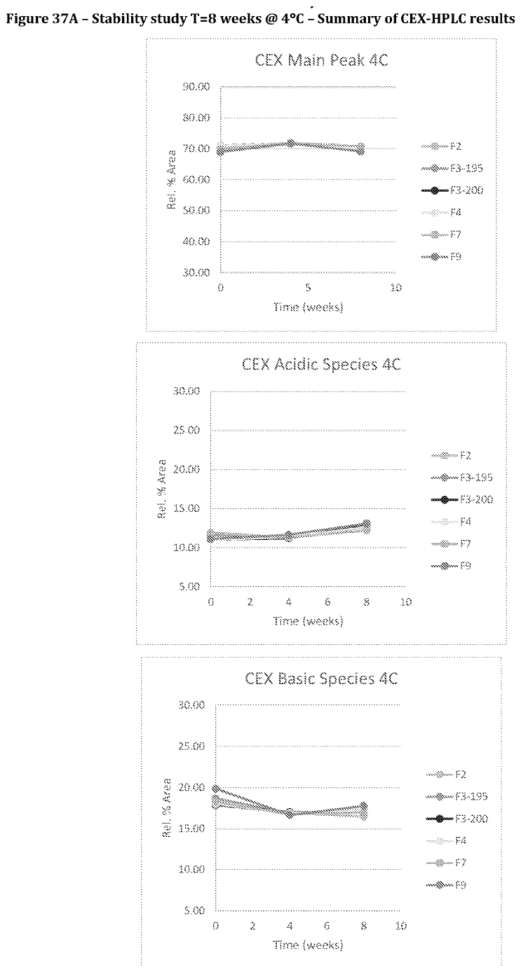

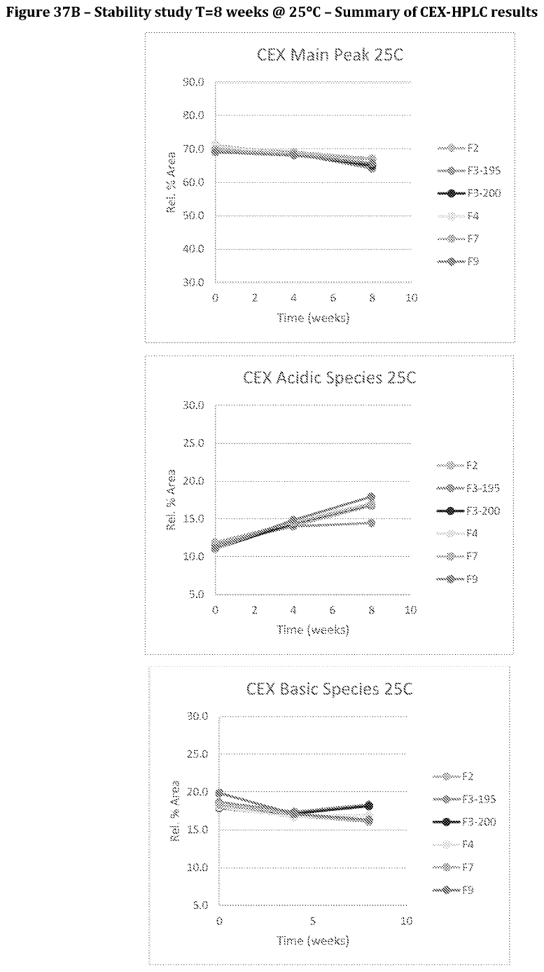

FIGS. 36 to 37 show the CEX-HPLC results at T=8 weeks. The results suggest that when stored at 4° C., all the formulations had excellent chemical stability at 8 weeks. When stored at 25° C., all the formulations had similar chemical stability after 8 weeks of storage.

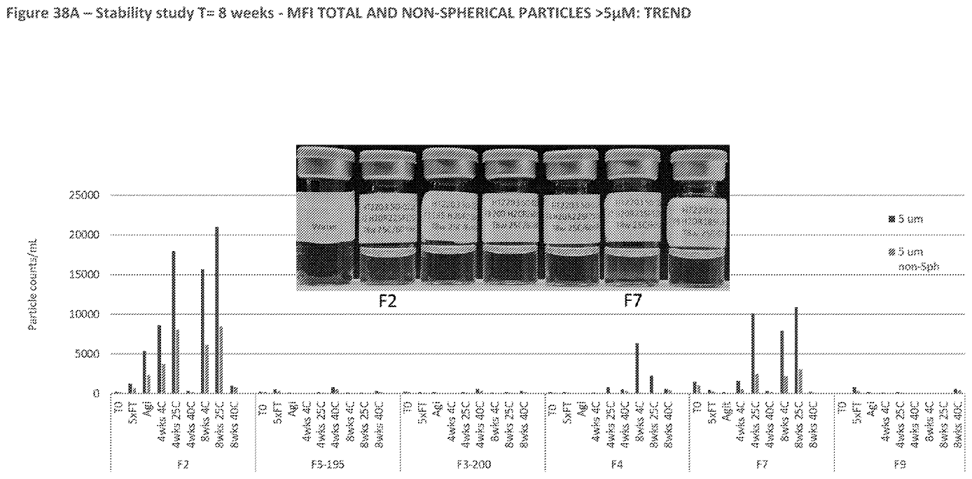

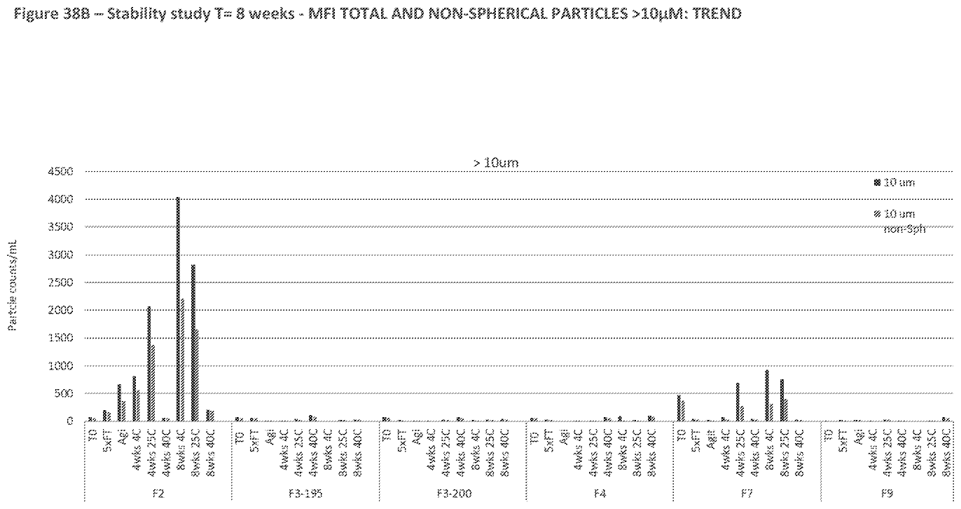

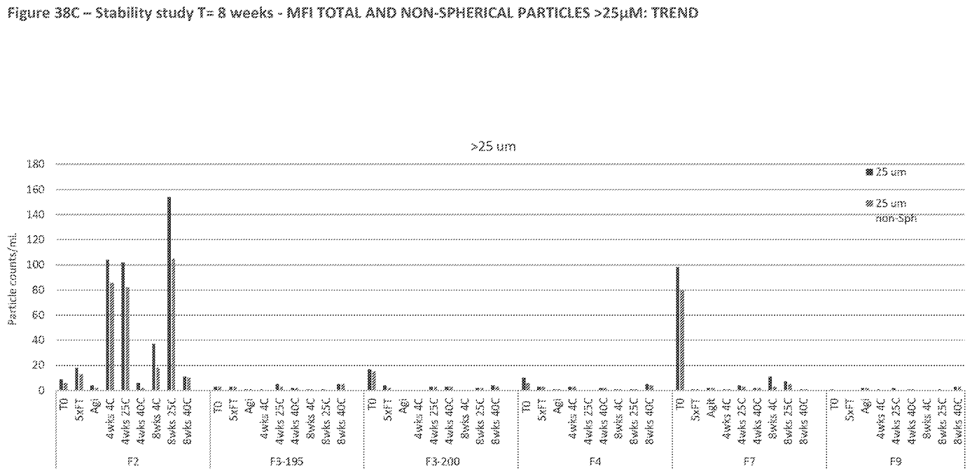

FIGS. 38A to 38C show the results of MFI) and non-SPH analysis. For the >5 μM size range, consistent with the results from T=4 weeks, formulation F2 showed the largest increase in subvisible particles (SVPs), followed by formulation F7. Visual observation suggest that SVP content in formulations F2 and F7 is strongly correlated with the formulation of visible particles (see FIGS. 28 to 29). At the 8-week time point, formulation F4 also exhibited an increase in SVP content. The same stability rankings were also observed for SVPs in the >10 μM and >25 μM size ranges.

Surprisingly, F3 and F9 show little to no increase in SVP content at every size range. Due to high sensitivity of the assay, such a result is rarely observed in formulations.

In summary, at the 8-week time point, leading formulations are beginning to emerge from the list of 5 formulation candidates—both F3 and F9 show promising stability, combined with acceptable viscosity.

The presence of tryptophan and its concentration appears to be important, as the formulation with no Trp (F2) was the least stable and the formulation with 20 mM Trp (F7) had lower stability during storage compared to the other formulations which had 50 mM Trp. HPLC data also appeared to suggest that, at the 8-week timepoint, overall stability of the 200 mg/mL eblasakimab formulations (in terms of both physical and chemical stability) was comparable to the previously developed 100 mg/ml eblasakimab formulation (JHL formulation).

Importantly, the stability of formulation F3 was demonstrated at both 195 mg/mL and 200 mg/mL, which boosts confidence in the results. Hence, based on the robust stability data, simplicity, and lack of contraindications, formulation F3 (20 mM His, 260 mM Arg, 50 mM Trp, 0.02% PS20) appears to be the preferred formulation candidate going forward.

### Example 5—Stability Study Results at 6 Months (26 Weeks)

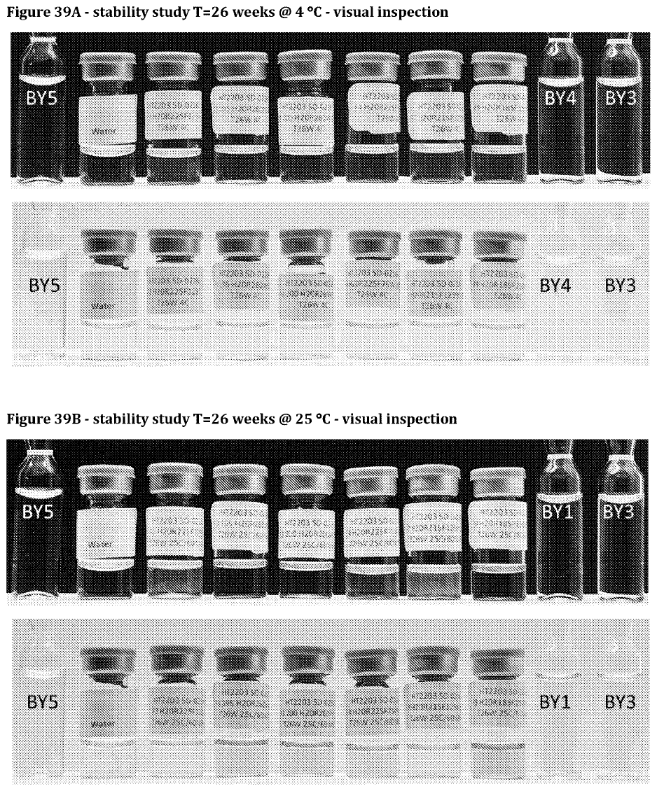

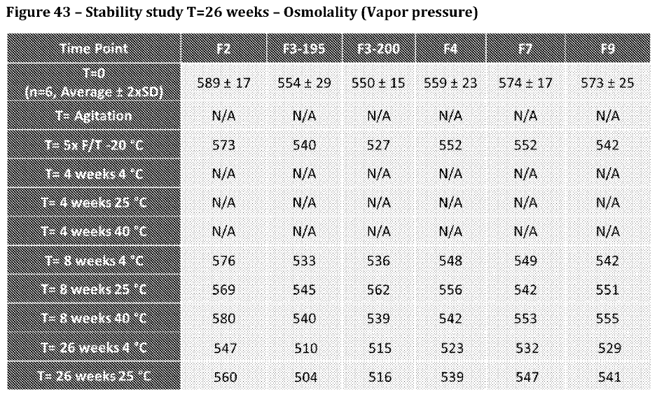

After 26 weeks of storage (T=26 weeks), the stability of the 5 formulations was further assessed by performing similar tests to those described in Example 4. The results of the tests are shown in FIGS. 39 to 50.

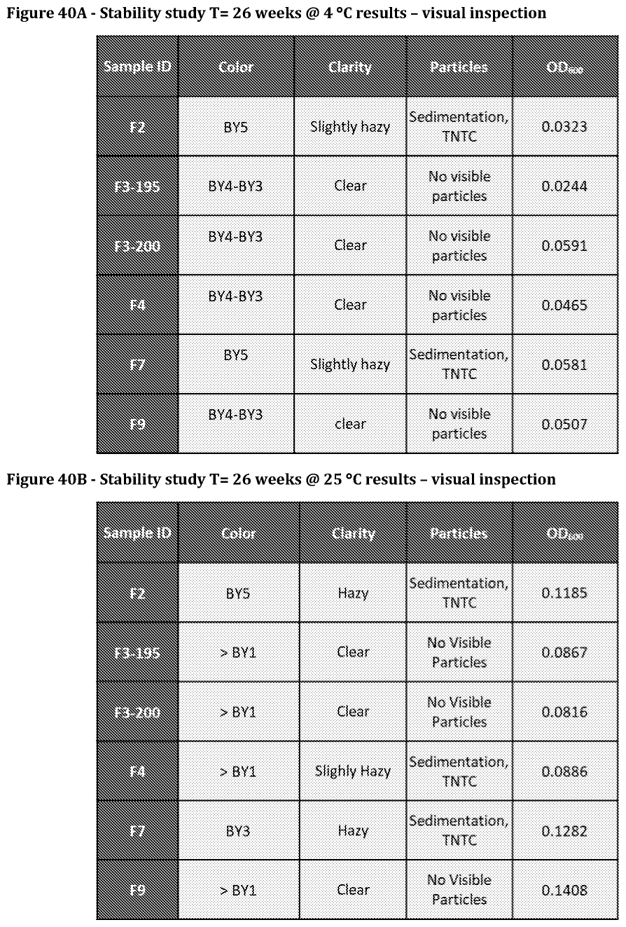

FIGS. 39 to 40 show the results of the visual inspection. As compared to the 8-week results, a large number of fine particles were now visible in both formulations F2 and F7 when stored at 4° C. and in formulations F2, F4 and F7 when stored at 25° C. The remaining formulations had no visible particles.

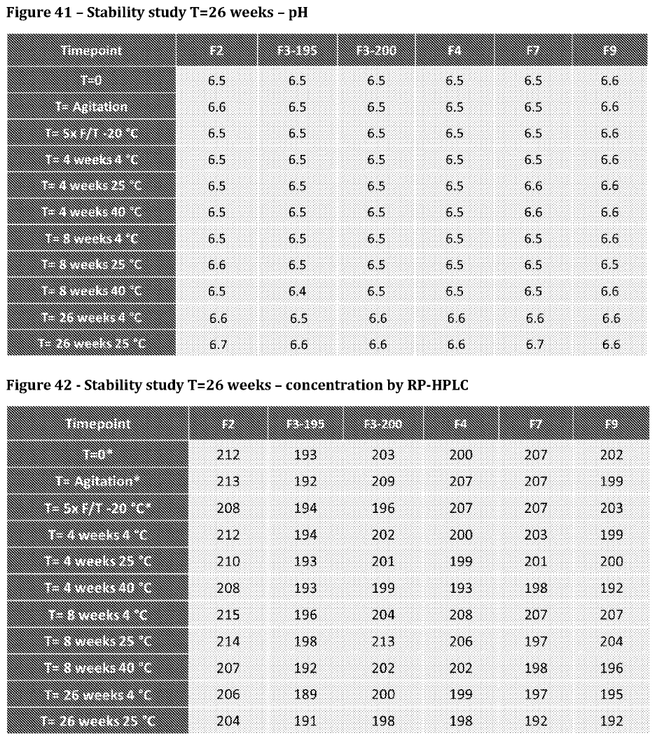

FIGS. 41 to 43 show the pH, concentration and osmolality of the formulations when measured at T=0, T=4 weeks, T=8 weeks and T=26 weeks. The results indicate no significant changes in the pH, concentration and osmolality over the course of the 26-week study, indicating that these aspects of the formulations continue to remain stable.

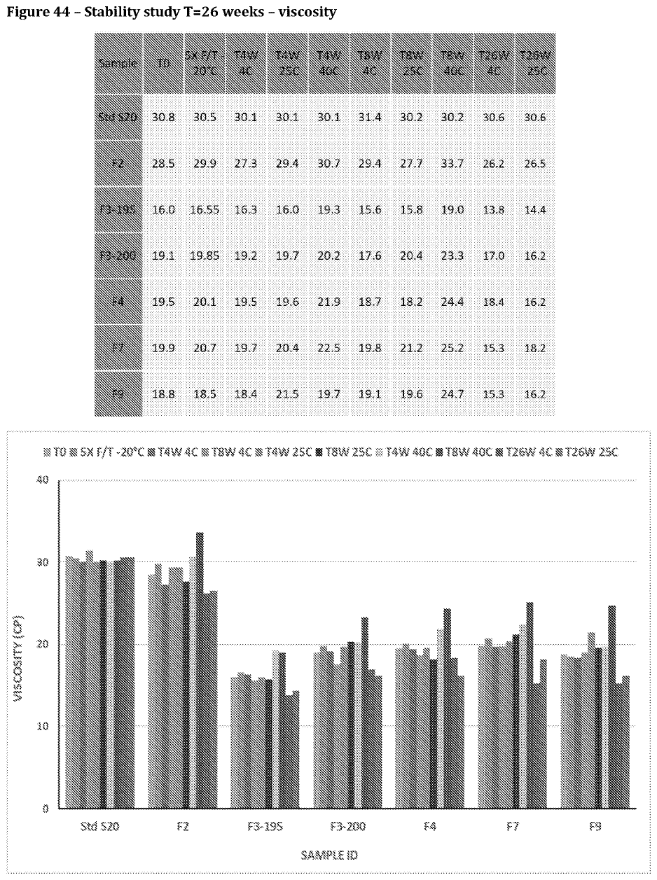

FIG. 44 shows the viscosity results for the formulations when measured at T=0, T=4 weeks, T=8 weeks, and T=26 weeks. With the exception of formulation F2, all the other formulations had viscosity readings close to or below the target of 20 cP at 8 weeks when stored under at 4° C. and 25° C. Formulation F3 continued to have the best viscosity.

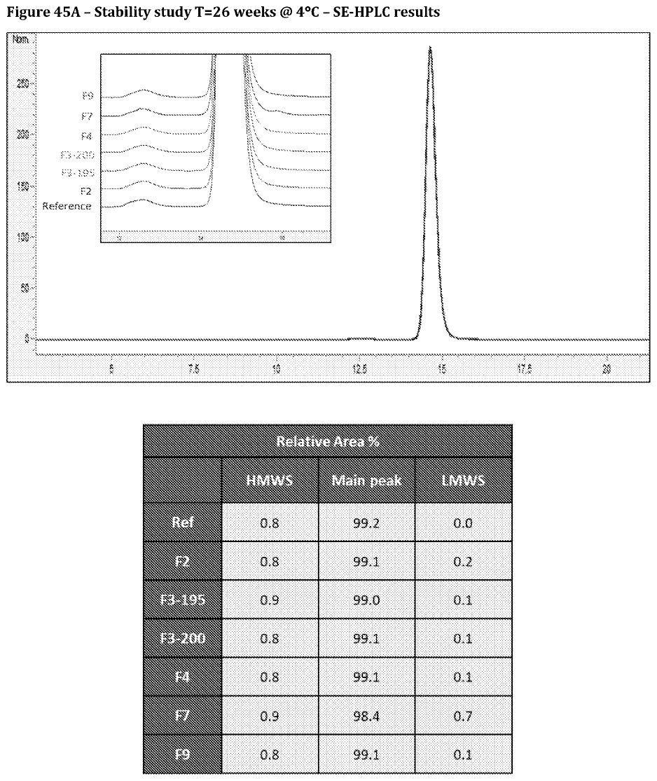

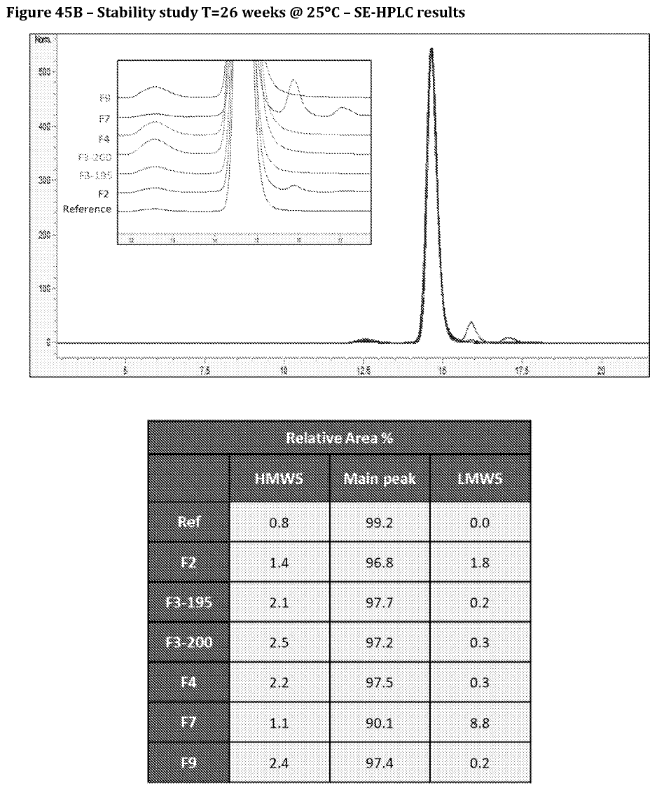

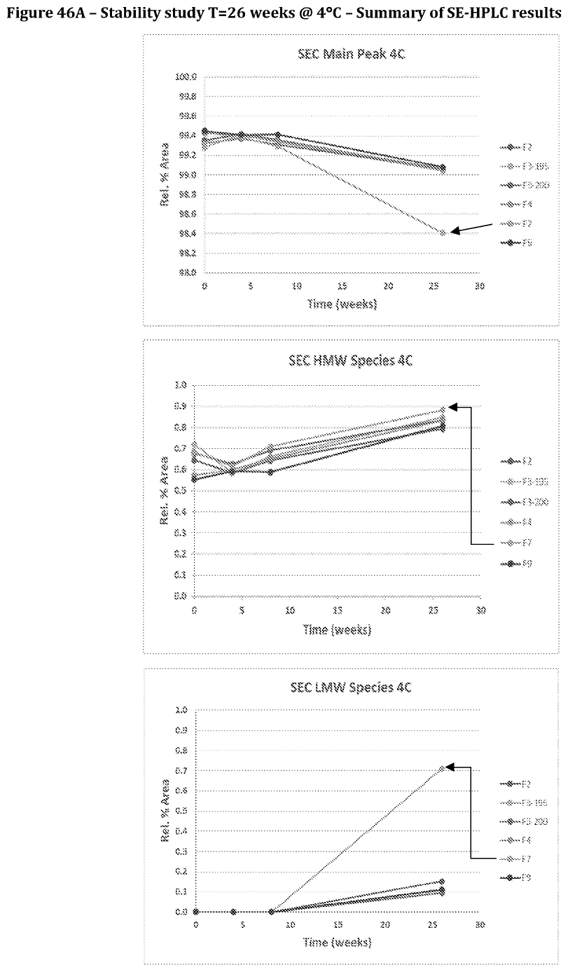

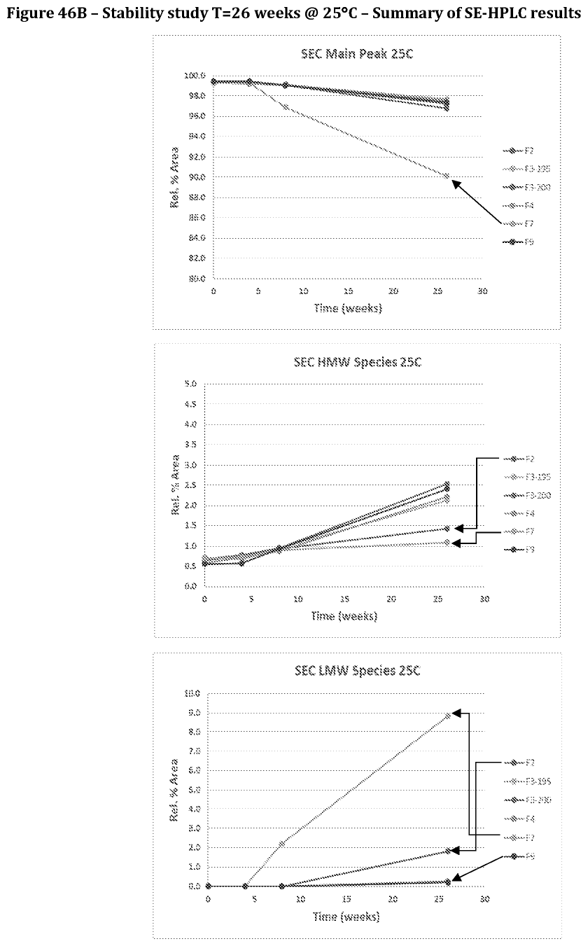

FIGS. 45 to 46 show the SE-HPLC results at T=26 weeks. The results suggest that when stored at 4° C., only formulation F7 had a significant loss in purity at 26 weeks. When stored at 25° C., all the formulations, with the exception of formulation F7, showed minimal loss of purity at 26 weeks.

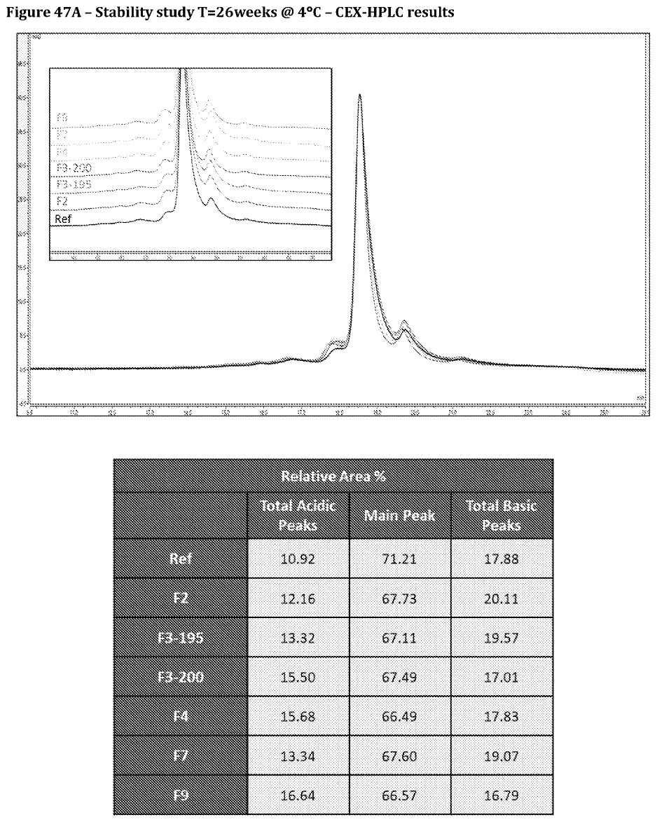

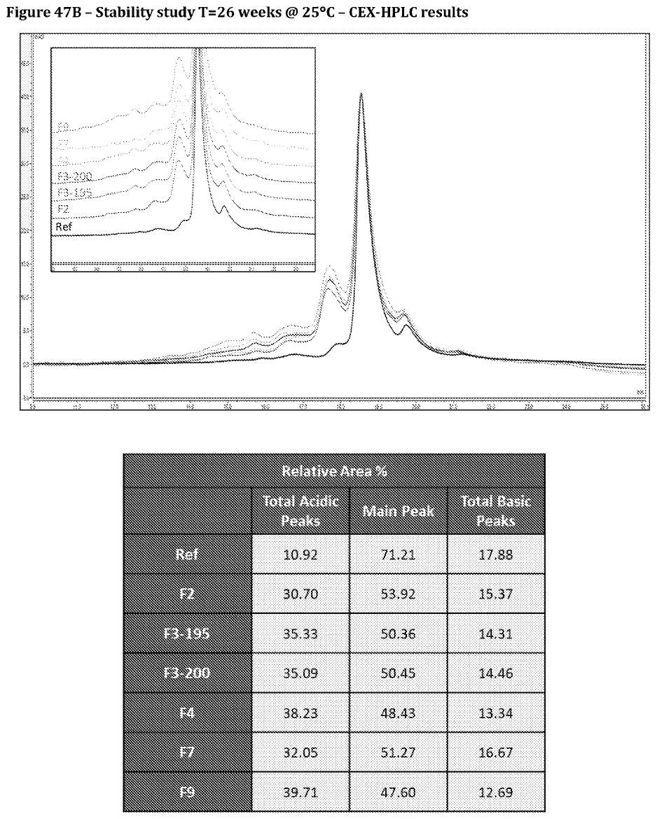

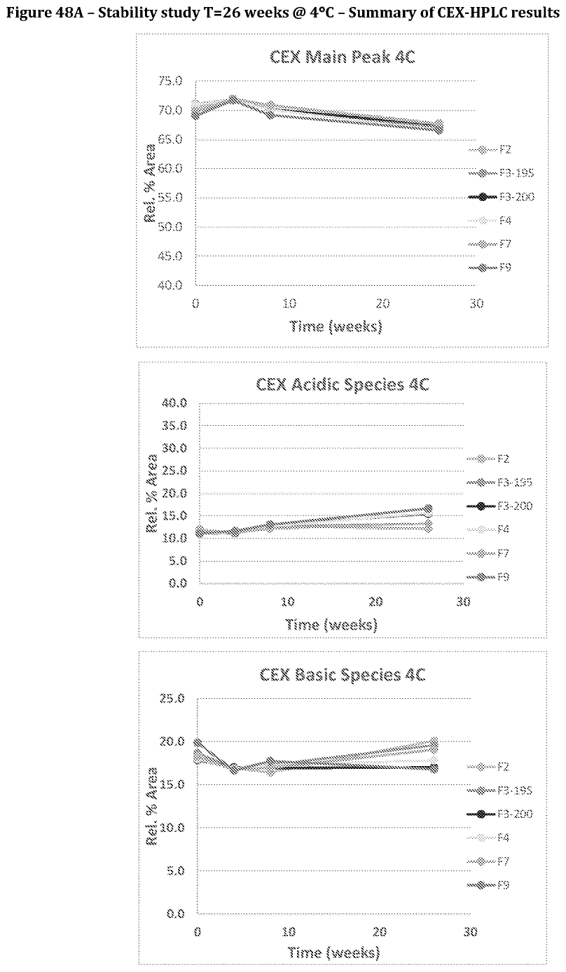

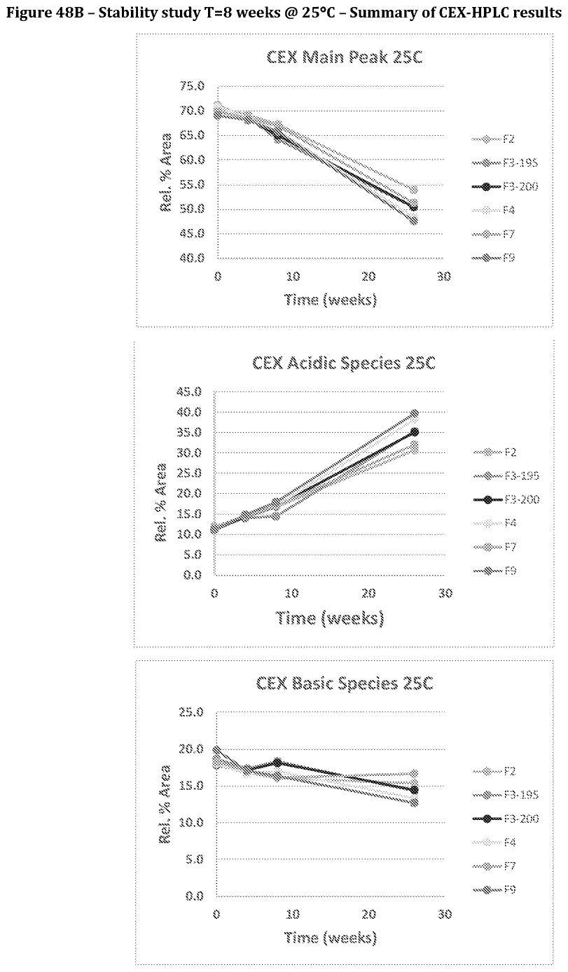

FIGS. 47 to 48 show the CEX-HPLC results at T=26 weeks. The results suggest that when stored at 4° C., all the formulations had excellent chemical stability at 26 weeks. When stored at 25° C., all the formulations had similar chemical stability after 26 weeks of storage.

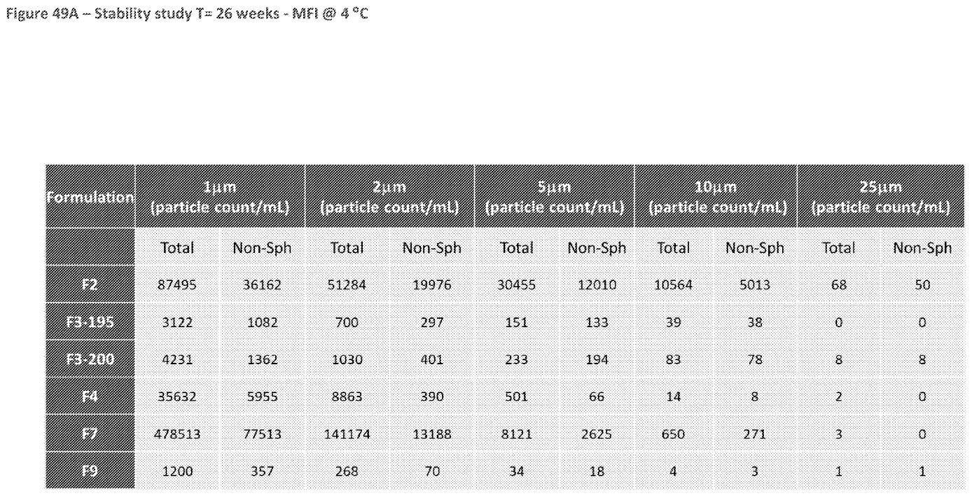

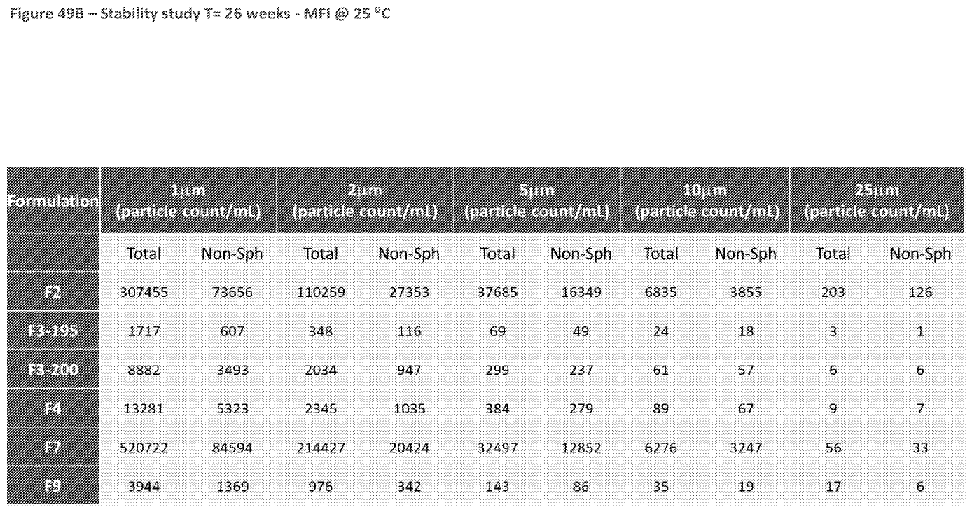

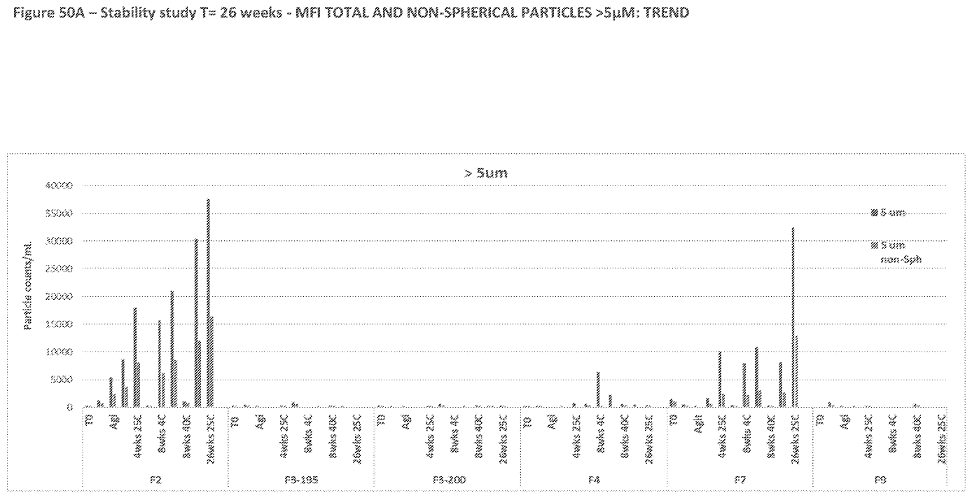

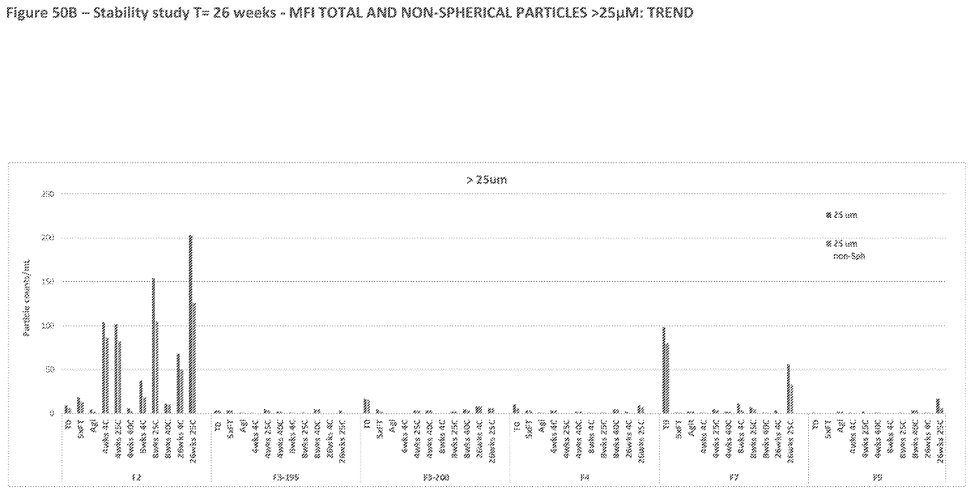

FIGS. 49 to 50 show the results of MFI) and non-SPH analysis. For the >5 μM size range, consistent with the results from T=8 weeks, formulation F2 showed the largest increase in subvisible particles (SVPs), followed by formulation F7.

In summary, at the 26 week time point, the results demonstrate that formulations F3 and F9 continue to have the best stability. Impressively, there were no signs of visible particle formation in F3 and F9 even after prolonged exposure to accelerated thermal stress conditions (25° C.).

The viscosity of all the formulations remains stable after 6 months of storage at 2-8° C. and 25° C. No significant difference was observed in the rate of chemical degradation (deamidation) between F3 and F9. F3 and F9 further show a very low propensity for particle formation. SVP content suggests a strong correlation with visible particle formation (see for example F2 and F7).

In conclusion, the stability results at T=26 weeks lends further support to formulation F3 as the lead formulation given its excellent stability at 4° C. and even at 25° C.

### Example 6—Formulation 1

Formulation 1 is: 200 mg/ml eblasakimab, 20 mM histidine, 150 mM arginine, 80 mM tryptophan, 0.02% Tween 20, wherein the pH of the formulation is pH 5.8.

### Example 7—Formulation 2

Formulation 2 is: 200 mg/ml eblasakimab, 50 mM histidine, 150 mM arginine, 20 mM tryptophan, 0.02% Tween 20, wherein the pH of the formulation is pH 6.8.

### Example 8—Formulation 3

Formulation 3 is: 200 mg/ml eblasakimab, 20 mM histidine, 150 mM arginine, 20 mM tryptophan, 0.02% Tween 20, wherein the pH of the formulation is pH 5.8.

### Example 9—Formulation 4

Formulation 4 is: 200 mg/ml eblasakimab, 50 mM histidine, 280 mM arginine, 20 mM tryptophan, 0.02% Tween 20, wherein the pH of the formulation is pH 5.8.

### Example 10—Formulation 5

Formulation 5 is: 200 mg/ml eblasakimab, 50 mM histidine, 215 mM arginine, 80 mM tryptophan, 0.02% Tween 20, wherein the pH of the formulation is pH 6.3.

### Example 11—Formulation 6

Formulation 6 is: 200 mg/ml eblasakimab, 20 mM histidine, 280 mM arginine, 80 mM tryptophan, 0.02% Tween 20, wherein the pH of the formulation is pH 6.8.

### Example 12—Formulation 7

Formulation 7 is: 200 mg/ml eblasakimab, 20 mM histidine, 280 mM arginine, 20 mM tryptophan, 0.02% Tween 20, wherein the pH of the formulation is pH 6.8.

### Example 13—Formulation 8

Formulation 8 is: 200 mg/ml eblasakimab, 20 mM histidine, 280 mM arginine, 50 mM tryptophan, 0.02% Tween 20, wherein the pH of the formulation is pH 5.8.

### Example 14—Formulation 9

Formulation 9 is: 200 mg/ml eblasakimab, 35 mM histidine, 280 mM arginine, 80 mM tryptophan, 0.02% Tween 20, wherein the pH of the formulation is pH 6.3.

### Example 15—Formulation 10

Formulation 10 is: 200 mg/ml eblasakimab, 20 mM histidine, 150 mM arginine, 20 mM tryptophan, 0.02% Tween 20, wherein the pH of the formulation is pH 5.8.

### Example 16—Formulation 11

Formulation 11 is: 200 mg/ml eblasakimab, 20 mM histidine, 150 mM arginine, 50 mM tryptophan, 0.02% Tween 20, wherein the pH of the formulation is pH 6.8.

### Example 17—Formulation 12

Formulation 12 is: 200 mg/ml eblasakimab, 50 mM histidine, 150 mM arginine, 20 mM tryptophan, 0.02% Tween 20, wherein the pH of the formulation is pH 6.3.

### Example 18—Formulation 13

Formulation 13 is: 200 mg/ml eblasakimab, 50 mM histidine, 280 mM arginine, 50 mM tryptophan, 0.02% Tween 20, wherein the pH of the formulation is pH 6.8.

### Example 19—Formulation 14

Formulation 14 is: 200 mg/ml eblasakimab, 20 mM histidine, 150 mM arginine, 80 mM tryptophan, 0.02% Tween 20, wherein the pH of the formulation is pH 5.8.

### Example 20—Formulation 15

Formulation 15 is: 200 mg/ml eblasakimab, 20 mM histidine, 150 mM arginine, 80 mM tryptophan, 0.02% Tween 20, wherein the pH of the formulation is pH 6.3.

### Example 21—Formulation 16

Formulation 16 is: 200 mg/ml eblasakimab, 35 mM histidine, 150 mM arginine, 80 mM tryptophan, 0.02% Tween 20, wherein the pH of the formulation is pH 5.8.

### Example 22—Formulation 17

Formulation 17 is: 200 mg/ml eblasakimab, 20 mM histidine, 215 mM arginine, 20 mM tryptophan, 0.02% Tween 20, wherein the pH of the formulation is pH 6.8.

### Example 23—Formulation 18

Formulation 18 is: 200 mg/ml eblasakimab, 50 mM histidine, 280 mM arginine, 80 mM tryptophan, 0.02% Tween 20, wherein the pH of the formulation is pH 5.8.

### Example 24—Formulation 19

Formulation 19 is: 200 mg/ml eblasakimab, 20 mM histidine, 260 mM arginine, 50 mM tryptophan, 0.02% Tween 20, wherein the pH of the formulation is pH 6.3.

### Example 25—Formulation 20

Formulation 20 is: 200 mg/ml eblasakimab, 20 mM histidine, 260 mM arginine, 50 mM tryptophan, 0.02% Tween 20, wherein the pH of the formulation is pH 6.8.

### Example 26—Formulation 21

Formulation 21 is: 200 mg/ml eblasakimab, 50 mM histidine, 150 mM arginine, 80 mM tryptophan, 0.02% Tween 20, wherein the pH of the formulation is pH 6.8.

### Example 27—Formulation 22

Formulation 22 is: 200 mg/ml eblasakimab, 20 mM histidine, 280 mM arginine, 20 mM tryptophan, 0.02% Tween 20, wherein the pH of the formulation is pH 6.8.

### Example 28—Formulation 23

Formulation 23 is: 200 mg/ml eblasakimab, 50 mM histidine, 150 mM arginine, 50 mM tryptophan, 0.02% Tween 20, wherein the pH of the formulation is pH 5.8.

### Example 29—Formulation 24

Formulation 24 is: 200 mg/ml eblasakimab, 35 mM histidine, 280 mM arginine, 20 mM tryptophan, 0.02% Tween 20, wherein the pH of the formulation is pH 5.8.

### Example 30—Formulation 25

Formulation 25 is: 200 mg/ml eblasakimab, 35 mM histidine, 215 mM arginine, 80 mM tryptophan, 0.02% Tween 20, wherein the pH of the formulation is pH 6.8.

### Example 31—Design of Experiment (DOE) Study

The objective of the design of experiment (DOE) study is to evaluate the effect of excipients on viscosity of eblaskaimab at the 200 mg/mL concentration in order to determine the optimal formulation design space.

### Materials and Methods

Table 3 below shows the details of the bulk drug substance (BDS) and the formulation parameters.

TABLE 3
BDS and Formulation parameters

| | |
|---|---|
| Bulk Drug Substance | ASLAN004/EB2002/JHL2221 Batch number |
| (BDS): | 212009 100 mg/mL in 20 mM His, 100 mM Arg, |
| | 180 mM sucrose, 0.02% Tween 20, pH 6.5 |
| Primary Container: | N/A |
| Formulation Buffers: | 20 mM Histidine |
| Formulation excipients: | Arginine, Tryptophan |
| Formulation Surfactants: | Tween 20 at 0.02% w/v |
| Concentration: | 200 mg/mL (After buffer exchange |
| | and concentrating) |
| Target Prep: | 700 μl |

The 25 formulations listed in Examples 5 to 30 above will be prepared for the DOE study. The complete list of formulations for the DOE study is shown in Table 4 below. 4 Parameters will be tested:
1. pH5.8 to 6.8
2. Histidine concentration 20 to 50 mM
3. Arginine concentration 150 to 280 nM
4. Tryptophan concentration 20 to 80 mM

TABLE 4
Target Formulation for the DoE (shaded rows denote
formulations that are replicated for enhancing statistical power)

| Formulation | pH | Histidine | Arginine | Tryptophan |
|---|---|---|---|---|
| 1 | 5.8 | 20 | 150 | 80 |
| 2 | 6.8 | 50 | 150 | 20 |
| 3 | 5.8 | 20 | 150 | 20 |
| 4 | 5.8 | 50 | 280 | 20 |
| 5 | 6.3 | 50 | 215 | 80 |
| 6 | 6.8 | 20 | 280 | 80 |
| 7 | 6.8 | 20 | 280 | 20 |
| 8 | 5.8 | 20 | 280 | 50 |
| 9 | 6.3 | 35 | 280 | 80 |
| 10 | 5.8 | 20 | 150 | 20 |
| 11 | 6.8 | 20 | 150 | 50 |
| 12 | 6.3 | 50 | 150 | 20 |
| 13 | 6.8 | 50 | 280 | 50 |
| 14 | 5.8 | 20 | 150 | 80 |
| 15 | 6.3 | 20 | 150 | 80 |
| 16 | 5.8 | 35 | 150 | 80 |
| 17 | 6.8 | 20 | 215 | 20 |
| 18 | 5.8 | 50 | 280 | 80 |
| 19 | 6.3 | 20 | 260 | 50 |
| 20 | 6.8 | 20 | 260 | 50 |
| 21 | 6.8 | 50 | 150 | 80 |
| 22 | 6.8 | 20 | 280 | 20 |
| 23 | 5.8 | 50 | 150 | 50 |
| 24 | 5.8 | 35 | 280 | 20 |
| 25 | 6.8 | 35 | 215 | 80 |

### Sample Handling and Preparation Process

1. An appropriate amount (35 mL) of BDS will be transferred in Spectra/Pro 1 dialysis Membrane (MWCO 6-8 kD) and will be dialyzed against 2×1000 L of 20 mM histidine, 200 mM Arginine at pH 6.5 first to remove sucrose in the buffer. The Drug Product (DP) formulation contains 0.02% Tween 20 so the surfactant will need to be spiked in after dialysis/ultrafiltration since the dialysis buffer does not contain Tween 20. The pH for the lower pH and higher pH formulations will be adjusted by adding an appropriate amount of 1 M HCl and/or NaOH.
2. The dialyzed protein sample will be concentrated to ~200 mg/mL by ultrafiltration using an Amicon Ultra −15 30,000 MWCO centrifugal concentrator at 3000 RPM using a Beckman 6KR refrigerated centrifuge.
3. The protein concentration in solutions will be determined by (A₂₈₀) using SoloVPE. If the protein concentration is below target, the bulk solution will be further concentrated by ultrafiltration until the target concentration is attained. In case the protein concentration is above 205 mg/mL, an appropriate amount of formulation buffer (20 mM his, 200 mM Arg at pH 6.5) can be added to reach the target concentration. Once the concentration of the dialyzed bulk drug substance has been confirmed to be ~200 mg/mL, then an appropriate amount of 10% Polysorbate 20 (PS20) will be spiked in the protein solution to make the final protein solution contain ~200 mg/mL eblasakimab, 20 mM Histidine, 200 mM Arginine, and 0.02% PS20 at pH 6.5.
4. The protein concentration will checked again after spiking in the PS20.
5. The concentrated protein solution will be aliquoted into 25 individual 2 mL glass vials that contain solid excipients listed in Table 3 to reach target formulation parameters listed in Table 4. The concentrated protein solution will be added to excipient solid in each individual vial then mixed by gentle pipetting up and down/swirling (do not vortex the sample).
6. After the solid excipient is completely dissolved and has formed a homogenous solution, the sample is left at 4-8 C overnight to let any air bubbles settle.
7. The next day, each sample is visually inspected in a light box and a photo is taken of the sample with a black and white background to document the samples' physical appearance, with a particular focus on the level of turbidity and presence of particles. Only the formulations that are free of visible particulates and/or phase separation will be utilized for further testing.
8. Following visual inspection, the samples that pass the inspection will be transferred to a 1.5 mL Eppendorf tube, and then spun down in a centrifuge at 10K RPM for 10 mins to remove any air bubbles before further analysis.
9. The supernatants from each sample will be carefully removed and then analysed by various assays in the following order: 1. pH; 2. Osmolality; 3. Viscosity.

The acceptance criteria will be ±0.1 for the pH. Osmolality should deviate <10% from theoretical osmolality values. Where a formulation fails to meet the specifications for pH, or target osmolality, the sample will not be subjected to viscosity measurements. Instead, the formulation will be re-prepared until it meets all target formulation parameters and specifications.

### Analysis of Samples

Samples will be evaluated using the analytical techniques, and in the order shown in Table 5 below:

TABLE 5
Summary of analytical techniques

| Analytical | | |
|---|---|---|
| Method | Purpose | Instrument |
| Visual | Appearance | Light box with black and white background |
| Inspection | and Clarity | |
| pH | pH | Mettler Toledo Seven Easy multiparameter |
| Osmolality | isotonicity | WESCOR Vapor pressure Osmometer |
| | | 500/Fiske Micro-Osmometer 210 |
| Viscosity | Viscosity | Brookfield DV-I viscometer/RheoSense μVISC |

At the conclusion of the experiments, the DoE analysis will be performed with 1 response (viscosity). The target viscosity is 20 cP.

The optimal formulation design space will be identified along with the key formulation parameters that influence viscosity, as well as any interactions between these parameters. The expected output will include Pareto plots, 3D-representations of the data, and 2D-contour plots showing the optimal design space for the response:

Pareto plots: gives ranking of variables tested (i.e. pH, histidine concentration, arginine concentration and tryptophan concentration). Analysis indicates which formulation variable is most important.

3D response surface plots: shows effects under different conditions. Reveals relative performance of different formulations under an identical set of experimental conditions and provides an empirical view of the design space.

Optimisation plots: helps identify formulations that truly optimise the viscosity of the eblasakimab 200 mg/ml formulations.

Danny Chou et al, Formulation and Development Review September/October 2016, Automated and rapid methods to assess quality and stability of biologics: recent developments and practical ways to implement them in formulation development, includes a review of the DOE approach and summarises the type of data output that can be generated as well as how the data can be used to determine the optimal design space.

### High Concentration Accelerated Stability Study

After successful completion of the viscosity screening, all formulations that meet the target viscosity (20 cP) will be further evaluated in a stability study. Samples will be analysed using the assays and conditions shown in Table 6.

TABLE 6
Summary of stability screening conditions

| Storage Timepoints | T0 | T2 wk | T4 wk | T8 wk |
|---|---|---|---|---|
| No. of conditions | 6 | 6 | 6 | 6 |
| 2-8° C. | X | X | X | X |
| 25° C./60% r.h. | | X | X | X |
| 45° C./75% r.h. | | X | X | |

Analytical methods

| | | | | |
|---|---|---|---|---|
| HP-SEC | ✓ | ✓ | ✓ | ✓ |
| CIEX-HPLC | ✓ | ✓ | ✓ | ✓ |
| Visual inspection | ✓ | ✓ | ✓ | ✓ |

1. A high concentration formulation of an anti-IL-13R antibody or an antigen binding fragment thereof, said formulation comprising:
175 to 250 mg/ml of an anti-IL-13R antibody or antigen binding fragment thereof, for example 175, 180, 185, 190, 195, 200, 205, 210, 215, 220, 225, 230, 235, 240, 245 or 250 mg/ml, in particular 190, 200 mg/ml, 210 mg/ml, 225 mg/ml or 250 mg/ml, such as 200 mg/ml of an anti-IL-13R antibody or antigen binding fragment thereof;
5 to 100 mM of tryptophan (including 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mM), for example 15 to 75 mM of tryptophan, such as 15 to 60 mM, in particular 25 to 50 mM tryptophan, such as 20 mM, 50 or 80 mM tryptophan;
140 to 290 mM of arginine, for example 160 to 290 mM of arginine (such as Arg-HCl or Arg-Glu), for example 160, 165, 175, 180, 185, 190, 195, 200, 205, 210, 215, 220, 225, 230, 235, 240, 245, 250, 255, 260, 265, 270, 275, 280 285 or 290 mM, in particular 150, 185, 215, 225, 250, 260 or 280 mM arginine;
0.01-0.03% of a non-ionic surfactant (such as polysorbate), for example 0.01-0.03% w/w, such as 0.01%, 0.015%, 0.020%, 0.025% or 0.030% in particular 0.02% w/w of a non-ionic surfactant; a buffer (such as histidine buffer), for example 15 to 55 mM of a buffer, such as 15, 20, 25, 30, 35, 40, 45, 50 or 55 mM of a histidine buffer; in particular 20, 35 or 50 mM histidine buffer; and wherein the pH of the formulation is in the range 5.5 to 7.2, for example 6.0 to 7.0, such as 6.0, 6.1, 6.2, 6.3, 6.4, 6.5, 6.6, 6.7, 6.8, 6.9 or 7.0, in particular 5.8, 6.3, 6.4, 6.5, 6.6, 6.7 or 6.8.

2. The formulation according to claim 1, said formulation comprising:
190 to 210 mg/ml of an anti-IL-13R antibody or antigen binding fragment thereof, for example 190, 195, 200, 205, or 210, in particular 200 mg/ml;
25 to 60 mM of tryptophan, such as 25, 30, 35, 40, 45, 50, 55, or 60 mM tryptophan, in particular 25 to 50 mM tryptophan;
175 to 270 mM of arginine (such as Arg-HCl or Arg-Glu), for example 190, 195, 200, 205, 210, 215, 220, 225, 230, 235, 240, 245, 250, 255, 260, 265 or 270 mM, in particular 200, 210, 225, 235, 250 or 260 mM;
0.01-0.03% of a non-ionic surfactant, for example 0.01-0.03% w/w, such as 0.02% w/w polysorbate 20 or polysorbate 80; a buffer (such as histidine buffer, for example 20 mM histidine buffer); and wherein the pH of the formulation is in the range 6.4 to 6.6, such as 6.4, 6.5 or 6.6.

3. The formulation according to claim 1, wherein the formulation comprises 190 to 210 mg/ml, such as 190, 195, 200, 205 or 210 mg/ml of the anti-IL13R antibody or a binding fragment thereof, for example 200 mg/ml of the anti-IL13R antibody or an antigen binding fragment thereof.

4. (canceled)

5. The formulation according to claim 1, 15 to 55 mM tryptophan, such as 15, 20, 25, 30, 35, 40, 45, 50 or 55 mM tryptophan, for example 20 or 40 mM tryptophan.

6. (canceled)

7. The formulation according to claim 1, wherein the formulation comprises 185 to 260 mM arginine, such as 185, 195, 205, 215, 225, 235, 245, 255 or 260 mM arginine, such as 185, 215, 225 or 260 mM arginine, for example 260 mM arginine, such as 260 mM arginine HCl.

8. (canceled)

9. (canceled)

10. The formulation according to claim 1, wherein the formulation comprises 25 to 175 mM phenylalanine, such as 25, 30, 35, 40, 45, 50, 55, 60, 65, 70, 75, 80, 85, 90, 95, 100, 105, 110, 115, 120, 125, 130, 135, 140, 145, 150, 155, 160, 165, 170 or 175 mM phenylalanine, such as 50 to 150 mM phenylalanine, for example 50, 55, 60, 65, 70, 75, 80, 85, 90, 95, 100, 105, 110, 115, 120, 125, 130, 135, 140, 145 or 150 mM phenylalanine, 50, 75, 100, 125 or 150 mM phenylalanine, such as 75, 125 or 150 mM phenylalanine, in particular 150 mM phenylalanine.

11. (canceled)

12. The formulation according to claim 1, wherein the formulation comprises 20 to 50 mM histidine buffer, for example 20, 25, 30, 35, 40, 45 or 50 mM, such as 20 mM or 50 mM histidine buffer, for example 20 mM histidine buffer.

13. (canceled)

14. The formulation according to claim 1, comprising 0.02% w/w of a non-ionic surfactant, optionally wherein the non-ionic surfactant is polysorbate, for example polysorbate 20, 40, 60, or 80, in particular 80, such as polysorbate 20.

15. (canceled)

16. (canceled)

17. The formulation according to claim 1, wherein the formulation has a viscosity in the range of 10 to 30 cP (mPa·s), for example 10, 11, 12, 13, 14, 15, 16, 17, 18, 19, 20, 21, 22, 23, 24, 25, 26, 27, 28, 29 or 30 cP, such as 15 to 25 cP, in particular 20 cP.

18. The formulation according to claim 1, wherein the formulation has a pH in the range of 6.4 to 6.7, such as 6.4, 6.5, 6.6 or 6.7.

19. The formulation according to claim 1, wherein the formulation is selected from one of the following groups:
(i) the formulation comprising:
260 mM of arginine-HCl;
50 mM tryptophan;
0.02% of a non-ionic surfactant, such as polysorbate 20; and
wherein the pH of the formulation is 6.3 to 6.7, such as 6.4, 6.5 or 6.6;
(ii) the formulation comprises:
225 mM of arginine-HCl;
75 mM phenylalanine; 50 mM tryptophan;
0.02% of a non-ionic surfactant, such as polysorbate 20; and
wherein the pH of the formulation is 6.3 to 6.7, such as 6.4, 6.5 or 6.6;
(iii) the formulation comprises:
215 mM of arginine-HCl;
125 mM phenylalanine;
20 mM tryptophan;
0.02% of a non-ionic surfactant, such as polysorbate 20; and
wherein the pH of the formulation is 6.3 to 6.7, such as 6.4, 6.5 or 6.6; and
(iv) the formulation comprises:
185 mM of arginine-HCl;
150 mM phenylalanine;
50 mM tryptophan;
0.02% of a non-ionic surfactant, such as polysorbate 20; and
wherein the pH of the formulation is 6.3 to 6.7, such as 6.4 or 6.5.

20. (canceled)

21. (canceled)

22. (canceled)

23. The formulation according to claim 1, wherein the anti-IL13R antibody or antigen binding fragment thereof comprises a VH domain comprising an amino acid sequence shown in SEQ ID NO: 51 or a sequence at least 95% identical thereto and a VL domain comprising an amino acid sequence shown in SEQ ID NO: 53 or a sequence at least 95% identical thereto.

24. The formulation according to claim 1, wherein the anti-IL13R antibody is eblasakimab (ASLAN004).

25. The formulation according to claim 1, wherein the formulation is selected from the group comprising:
Formulation 1 comprising 200 mg/ml eblasakimab, 20 mM histidine, 150 mM arginine, 80 mM tryptophan, and 0.02% Tween 20, wherein the pH of the formulation is 5.8;
Formulation 2 comprising 200 mg/ml eblasakimab, 50 mM histidine, 150 mM arginine, 20 mM tryptophan, and 0.02% Tween 20, wherein the pH of the formulation is 6.8;
Formulation 3 comprising 200 mg/ml eblasakimab, 20 mM histidine, 150 mM arginine, 20 mM tryptophan, and 0.02% Tween 20, wherein the pH of the formulation is 5.8;
Formulation 4 comprising 200 mg/ml eblasakimab, 50 mM histidine, 280 mM arginine, 20 mM tryptophan, and 0.02% Tween 20, wherein the pH of the formulation is 5.8;
Formulation 5 comprising 200 mg/ml eblasakimab, 50 mM histidine, 215 mM arginine, 80 mM tryptophan, and 0.02% Tween 20, wherein the pH of the formulation is pH 6.3;
Formulation 6 comprising 200 mg/ml eblasakimab, 20 mM histidine, 280 mM arginine, 80 mM tryptophan, and 0.02% Tween 20, wherein the pH of the formulation is pH 6.8;
Formulation 7 comprising 200 mg/ml eblasakimab, 20 mM histidine, 280 mM arginine, 20 mM tryptophan, and 0.02% Tween 20, wherein the pH of the formulation is pH 6.8;
Formulation 8 comprising 200 mg/ml eblasakimab, 20 mM histidine, 280 mM arginine, 50 mM tryptophan, and 0.02% Tween 20, wherein the pH of the formulation is pH 5.8;
Formulation 9 comprising 200 mg/ml eblasakimab, 35 mM histidine, 280 mM arginine, 80 mM tryptophan, and 0.02% Tween 20, wherein the pH of the formulation is pH 6.3;
Formulation 10 comprising 200 mg/ml eblasakimab, 20 mM histidine, 150 mM arginine, 20 M tryptophan, and 0.02% Tween 20, wherein the pH of the formulation is pH 5.8;
Formulation 11 comprising 200 mg/ml eblasakimab, 20 mM histidine, 150 mM arginine, 50 mM tryptophan, and 0.02% Tween 20, wherein the pH of the formulation is pH 6.8;
Formulation 12 comprising 200 mg/ml eblasakimab, 50 mM histidine, 150 mM arginine, 20 mM tryptophan, and 0.02% Tween 20, wherein the pH of the formulation is pH 6.3;
Formulation 13 comprising 200 mg/ml eblasakimab, 50 mM histidine, 280 mM arginine, 50 mM tryptophan, 0.02% Tween 20, wherein the pH of the formulation is pH 6.8;
Formulation 14 comprising 200 mg/ml eblasakimab, 20 mM histidine, 150 mM arginine, 80 mM tryptophan, and 0.02% Tween 20, wherein the pH of the formulation is pH 5.8;
Formulation 15 comprising 200 mg/ml eblasakimab, 20 mM histidine, 150 mM arginine, 80 mM tryptophan, and 0.02% Tween 20, wherein the pH of the formulation is pH 6.3;
Formulation 16 comprising 200 mg/ml eblasakimab, 35 mM histidine, 150 mM arginine, 80 mM tryptophan, and 0.02% Tween 20, wherein the pH of the formulation is pH 5.8;
Formulation 17 comprising 200 mg/ml eblasakimab, 20 mM histidine, 215 mM arginine, 20 mM tryptophan, and 0.02% Tween 20, wherein the pH of the formulation is pH 6.8;
Formulation 18 comprising 200 mg/ml eblasakimab, 50 mM histidine, 280 mM arginine, 80 mM tryptophan, and 0.02% Tween 20, wherein the pH of the formulation is pH 5.8;
Formulation 19 comprising 200 mg/ml eblasakimab, 20 mM histidine, 260 mM arginine, 50 mM tryptophan, and 0.02% Tween 20, wherein the pH of the formulation is pH 6.3;
Formulation 20 comprising 200 mg/ml eblasakimab, 20 mM histidine, 260 mM arginine, 50 mM tryptophan, and 0.02% Tween 20, wherein the pH of the formulation is pH 6.8;
Formulation 21 comprising 200 mg/ml eblasakimab, 50 mM histidine, 150 mM arginine, 80 mM tryptophan, and 0.02% Tween 20, wherein the pH of the formulation is pH 6.8;
Formulation 22 comprising 200 mg/ml eblasakimab, 20 mM histidine, 280 mM arginine, 20 mM tryptophan, and 0.02% Tween 20, wherein the pH of the formulation is pH 6.8;
Formulation 23 comprising 200 mg/ml eblasakimab, 50 mM histidine, 150 mM arginine, 50 mM tryptophan, and 0.02% Tween 20, wherein the pH of the formulation is pH 5.8;
Formulation 24 comprising 200 mg/ml eblasakimab, 35 mM histidine, 280 mM arginine, 20 mM tryptophan, and 0.02% Tween 20, wherein the pH of the formulation is pH 5.8; and
Formulation 25 comprising 200 mg/ml eblasakimab, 35 mM histidine, 215 mM arginine, 80 mM tryptophan, and 0.02% Tween 20, wherein the pH of the formulation is pH 6.8.

26. The formulation according to claim 1, wherein the formulation is stable at refrigerator temperature, for example at 8° C. or less, such as 2 to 8° C., such as 8, 7, 6, 5, 4, 3, 2, or 1° C., in particular 4° C.

27. The formulation according to claim 1, wherein the formulation is stable at room temperature, for example 15 to 25° C., such as 15, 16, 17, 18, 19, 20, 21, 22, 23, 24 or 25° C., in particular 25° C.

28. The formulation according to claim 1, wherein the formulation is stable for at least one month, such as at least 2, 3, 6, 12, 18, 24, 30 or 36 months.

29. The formulation according to claim 1, for use in treatment, for example for use in the treatment of inflammation or an autoimmune disease, such as for treating a chronic inflammation.

30. The formulation according to claim 1, for use in the treatment of atopic dermatitis.
